(19) United States
(12) Patent Application Publication
(10) Pub. No.: US 2012/0143206 A1
(43) Pub. Date: Jun. 7, 2012

(54) SURGICAL TOOLS FOR TREATMENT OF SPINAL STENOSIS

(76) Inventors: Michael P. Wallace, Pleasanton, CA (US); Roy Leguidleguid, Union City, CA (US); Ronald Leguidleguid, Union City, CA (US); Nestor C. Cantorna, Union City, CA (US)

(21) Appl. No.: 13/338,134

(22) Filed: Dec. 27, 2011

Related U.S. Application Data
(63) Continuation-in-part of application No. 12/824,043, filed on Jun. 25, 2010.
(60) Provisional application No. 61/220,314, filed on Jun. 25, 2009.
(60) Provisional application No. 61/253,811, filed on Oct. 21, 2009.
(60) Provisional application No. 61/427,449, filed on Dec. 27, 2010.

(51) Int. Cl. A61B 17/68

(52) U.S. Cl. 606/103

(57) ABSTRACT

Described herein are devices and methods for positioning a wire around a target tissue. In some embodiments, a probe device includes a rigid outer cannula having a curved distal region and a flexible inner cannula slideably disposed within the rigid outer cannula, wherein the inner cannula is configured to assume a curved shape when extended distally from the outer cannula. In some embodiments, the probe device further includes a distal tip at the distal end of the inner cannula and a safety retainer cable coupled to the distal tip and secured to the probe proximally from the distal end of the safety retainer; wherein the safety retainer cable extends proximally from the distal tip and is slack. In some embodiments, the inner cannula comprises an elongate body with a longitudinally extending support member and a longitudinally extending tubular body, configured to pass a wire, disposed inside the elongate body.

### CROSS REFERENCE TO RELATED APPLICATIONS

This patent application is a continuation-in-part of U.S. patent application Ser. No. 12/824,043, titled “SURGICAL TOOLS FOR TREATMENT OF SPINAL STENOSIS”, filed on Jun. 25, 2010, now Publication No. US-2010-0331900-A1, which claims priority to U.S. Provisional Patent Application No. 61/220,314, titled “SURGICAL TOOLS FOR TREATMENT OF SPINAL STENOSIS”, filed on Jun. 25, 2009 and U.S. Provisional Patent Application No. 61/253,811, titled “SURGICAL TOOLS FOR TREATMENT OF SPINAL STENOSIS”, filed on Oct. 21, 2009.

This patent application also claims priority to U.S. Provisional Patent Application No. 61/427,449, titled “TISSUE REMOVAL DEVICES AND METHODS”, filed on Dec. 27, 2010. These patent applications are each incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are systems, devices, and methods for performing surgical procedures. In particular, described herein are systems, devices and methods for spinal decompression procedures.

### BACKGROUND

In recent years, less invasive (or “minimally invasive”) surgical techniques have become increasingly more popular, as physicians, patients and medical device innovators have sought to achieve similar or improved outcomes, relative to conventional surgery, while reducing the trauma, recovery time and side effects typically associated with conventional surgery. Developing less invasive surgical methods and devices, however, can pose many challenges. For example, some challenges of less invasive techniques include working in a smaller operating field, working with smaller devices, and trying to operate with reduced or even no direct visualization of the structure (or structures) being treated. These challenges are compounded by the fact that target tissues to be modified often reside very close to one or more vital, non-target tissues, which the surgeon hopes not to damage. One of the initial obstacles in any given minimally invasive procedure, therefore, is positioning a minimally invasive surgical device in a desired location within the patient to perform the procedure on one or more target tissues, while avoiding damage to nearby non-target tissues.

Examples of less invasive surgical procedures include laparoscopic procedures, arthroscopic procedures, and minimally invasive approaches to spinal surgery, such as a number of less invasive intervertebral disc removal, repair and replacement techniques. One area of spinal surgery in which a number of less invasive techniques have been developed is the treatment of spinal stenosis. Spinal stenosis occurs when neural and/or neurovascular tissue in the spine becomes impinged by one or more structures pressing against them, causing one or more symptoms. This impingement of tissue may occur in one or more of several different areas in the spine, such as in the central spinal canal, or more commonly in the lateral recesses of the spinal canal and/or one or more intervertebral foramina.

One common cause of spinal stenosis is buckling and thickening of the ligamentum flavum (one of the ligaments attached to and connecting the vertebrae). Buckling or thickening of the ligamentum flavum may impinge on one or more neurovascular structures, dorsal root ganglia, nerve roots and/or the spinal cord itself. Another common cause of neural and neurovascular impingement in the spine is hypertrophy of one or more facet joints (or “zygopophaseal joints”), which provide articulation between adjacent vertebrae. Vertebral facet superior articular processes articulate with inferior articular processes of adjacent vertebra to form zygopophaseal joints. Other causes of spinal stenosis include formation of osteophytes (or “bone spurs”) on vertebrae, spondylolisthesis (sliding of one vertebra relative to an adjacent vertebra), facet joint synovial cysts, and collapse, bulging or herniation of an intervertebral disc into the central spinal canal. Disc, bone, ligament or other tissue may impinge on the spinal cord, the cauda equina, branching spinal nerve roots and/or blood vessels in the spine to cause loss of function, ischemia and even permanent damage of neural or neurovascular tissue. In a patient, this may manifest as pain, impaired sensation and/or loss of strength or mobility.

In the United States, spinal stenosis occurs with an incidence of between 4% and 6% of adults aged 50 and older and is the most frequent reason cited for back surgery in patients aged 60 and older. Conservative approaches to the treatment of symptoms of spinal stenosis include systemic medications and physical therapy. Epidural steroid injections may also be utilized, but they do not provide long lasting benefits. When these approaches are inadequate, current treatment for spinal stenosis is generally limited to invasive surgical procedures to remove ligament, cartilage, bone spurs, synovial cysts, cartilage, and bone to provide increased room for neural and neurovascular tissue. The standard surgical procedure for spinal stenosis treatment includes laminectomy (complete removal of the lamina of one or more vertebrae) or laminotomy (partial removal of the lamina), followed by removal (or “resection”) of the ligamentum flavum. In addition, the surgery often includes partial or occasionally complete facetectomy (removal of all or part of one or more facet joints). In cases where a bulging intervertebral disc contributes to neural impingement, disc material may be removed surgically in a discectomy procedure.

Removal of vertebral bone, as occurs in laminectomy and facetectomy, often leaves the effected area of the spine very unstable, leading to a need for an additional highly invasive fusion procedure that puts extra demands on the patient's vertebrae and limits the patient's ability to move. In a spinal fusion procedure, the vertebrae are attached together with some kind of support mechanism to prevent them from moving relative to one another and to allow adjacent vertebral bones to fuse together. Unfortunately, a surgical spine fusion results in a loss of ability to move the fused section of the back, diminishing the patient's range of motion and causing stress on the discs and facet joints of adjacent vertebral segments. Such stress on adjacent vertebrae often leads to further dysfunction of the spine, back pain, lower leg weakness or pain, and/or other symptoms. Furthermore, using current surgical techniques, gaining sufficient access to the spine to perform a laminectomy, facetectomy and spinal fusion requires dissecting through a wide incision on the back and typically causes extensive muscle damage, leading to significant post-operative pain and lengthy rehabilitation. Discectomy procedures require entering through an incision in the patient's abdomen and navigating through the abdominal anatomy to arrive at the spine. Thus, while laminectomy, facetectomy, discectomy, and spinal fusion frequently improve symptoms of neural and neurovascular impingement in the short term, these procedures are highly invasive, diminish spinal function, drastically disrupt normal anatomy, and increase long-term morbidity above levels seen in untreated patients. Although a number of less invasive techniques and devices for spinal stenosis surgery have been developed, these techniques still typically require removal of significant amounts of vertebral bone and, thus, typically require spinal fusion.

Recently, less invasive surgical methods and systems for treating spinal stenosis have been developed, including. For example, devices or systems for positioning less invasive devices in a patient for performing a less invasive procedure using pullwires have been developed. In particular, sharp-tipped (e.g., with tissue-penetrating distal ends) may be inserted from a first location, pass through the tissue (e.g., through or adjacent to a compressed spinal neural foramen), and extend back out of the tissue. The pullwire may be inserted into and through highly stenosed, or blocked, areas of the spine. It may be therefore be difficult to insert and/or deploy a pullwire into position, preferably adjacent to or around a target tissue. Thus, it would be particularly useful to provide systems and methods for performing the procedures described above that include components such as cannulated probes that address some of these concerns. At least some of these objectives will be met by the present invention.

The pull wire may then be used to pull devices (e.g., tissue modification devices, neural localization devices, etc.) into position and also to activate them—e.g., articulating them by pulling them back and forth. In some instances, the forces required to pull the pullwire for positioning or articulating a device coupled to the end of the pullwire may exceed be quite large (e.g., exceeding 10 pounds of force), making the pullwire difficult to grip. In addition, the distal end of the pullwire, which may be sharp, may present a hazard to the surgeon or others performing the procedure. Thus, it would be particularly useful to provide systems for performing the procedures described above that include components such as distal handles that address some of these concerns. At least some of these objectives will be met by the present invention.

Described herein are surgical systems, device and methods that may be particularly useful for treating spinal stenosis.

### SUMMARY OF THE DISCLOSURE

In general, the systems for treating spinal stenosis described herein may include a pullwire, a removable distal handle for a pullwire, a probe for inserting a pullwire around a target tissue, a tissue modification device for coupling to the proximal end of a pullwire, and a neural localization device for coupling to the proximal end of a pullwire. The tissue modification device, neural localization device, and probe devices may be similarly adapted for use as a system, and in particular may be adapted to indicate the orientation of the devices and to prevent rotation of the devices during operation. For example, these devices may include a handle having a front and back that is marked.

A pullwire may also be referred to as a guidewire (and thus a pullwire handle may be referred to as a guidewire handle).

For example, described herein are systems for treating spinal stenosis that include: a pullwire having a tissue-penetrating distal end; a cannulated probe comprising a curvable inner cannula that is slideably disposed within a curved outer cannula, the cannulated probe configured for positioning the pullwire around a target tissue; a removable handle configured to secure the distal end of the pullwire; and a tissue modification device configured to couple to the proximal end of the pullwire; wherein the tissue modification device and the cannulated probe both include a proximal handle having a flat front and further wherein the front is marked to indicate the front orientation.

The system may also include a neural localization device that is elongate and flexible and is configured to couple to the proximal end of the pullwire.

In some variations, the flat front of the proximal handles of the cannulated probe and the tissue modification device are marked by one or more of a color or texture.

Also described herein are probes for positioning a pullwire around a target tissue, the probe comprising: an outer cannula having a curved distal region; an inner cannula slideably disposed within the outer cannula, wherein the inner cannula is configured to assume a curved shape when extended distally from the outer cannula; and a proximal handle comprising a hand grip region coupled to the outer cannula and a plunger region coupled to the inner cannula; wherein the proximal end of the plunger region comprises a funneled port for insertion of a pullwire through the probe.

Any of the probe variations described herein may include one or more safety features. For example, any of these probes may include a safety features such as a retainer, preventing the device from breaking off while in use. A probe may be subject to mechanical force during operation which may result in fracture or failure of the probe; in a worst case situation, a portion (e.g., the distal tip region) may be broken off while the device is within the tissue. The devices described herein may be configured to prevent breaking and/or to retain portions of the probe that may be broken so that the entire probe may be retrieved from the patient.

The hand grip region of the proximal handle may comprise a flat front face configured to allow sighting down the length of the probe. The front face of the hand grip region may be marked to indicate the front. For example, the marking may comprise a texture and/or a color.

In some variations, the hand grip region of the proximal handle may comprise a concave region configured to allow the hand grip region to be held like a pencil.

The hand grip region may include a flared distal end configured to facilitate the application of force. The device may also include depth markings on the plunger region of the proximal handle. The hand grip region of the proximal handle may also be keyed to prevent rotation of the probe during use.

Also described herein are neural localization devices including: a flexible, elongate, ribbon-shaped body extending distally, wherein the body comprises a front side and a back side; a distal pullwire coupling member configured to couple to the proximal end of a pullwire; a first electrode coupled to the front side of the elongate, ribbon-shaped body and configured to stimulate adjacent nerve tissue; a second electrode coupled to the back side of the elongate, ribbon-shaped body and configured to stimulate adjacent nerve tissue; and a proximal handle coupled to the body, the handle having a front and a back and comprising a control for activating either the first or second electrode; the handle further comprising an indicator on both the front and back of the handle to indicate activation of either the first or second electrodes.

In some variations, the proximal handle may include a marking indicating the front of the handle. The marking may be a texture and/or a color. In some variations, the proximal handle is keyed to prevent rotation of the device during use.

Also described are tissue modification devices for removing tissue that include: an elongate body having a flexible tissue modification region, wherein the tissue modification region comprises a plurality of blades; a distal pullwire coupling member configured to couple to the proximal end of a pullwire; and a proximal handle coupled to the elongate body, the handle having a front and back side, and comprising one or more markings to distinguish the front and back sides; wherein the proximal handle is keyed to prevent rotation of the device during use.

The proximal handle may be marked to indicate the orientation of the blades at the distal end of the device. The proximal handle may include a flared proximal end to enhance grip and provide leverage.

In some variations, the tissue modification region may be substantially ribbon-shaped, having a front and a back corresponding to the front and the back of the proximal handle. The markings may comprise a texture and/or a color. The proximal handle may be further marked to indicate the size or caliber of the tissue modification device.

Also described herein are pullwire handle devices for securing to the distal end of a tissue-penetrating pullwire that include: a handle body; a pullwire tip capture region (e.g., lever, chamber, etc.) configured to secure to a pullwire and to slideably move relative to the handle body for choking up on the pullwire within the handle body; a pullwire lock configured to lock the pullwire within the handle; and a wire capture indicator configured to indicate when a wire has been secured in the handle. In some variations, the devices further include a funnel-shaped pullwire feed region configured for inserting the distal end of a pullwire within the handle body.

The pullwire lock may comprise a button or lever configured to active and/or inactivate the lock. In some variations, the device includes an internal track for guiding the movement of the pullwire within the handle body.

The wire capture indicator may comprise a window showing a portion of a captured pullwire.

Also described herein are methods of capturing a pullwire using a pullwire handle device configured to secure to the distal end of a pullwire including the steps of inserting the distal end of a pullwire into the pullwire handle device; engaging the distal end of the pullwire with a pullwire tip capture region on the pullwire handle device; locking the pullwire within the handle; and indicating that a pullwire has been secured within the pullwire handle device. The pullwire handle may be moved along (or relative to) the pullwire after the distal tip region of the pullwire has been secured in the tip capture region (e.g., to “choke up” on the pullwire). The handle may then be locked down to secure the two together to prevent further relative movement between the two, although in some variations, the handle may be locked down onto the pullwire by activating a control (e.g., button, lever, etc.) but activated to secure together more tightly by pulling the pullwire slightly relative to the handle to further lock them together. An additional slight movement of the pullwire relative to the handle thus tightens the lock between the two (e.g., by a camming mechanism).

Securing the distal end of the pullwire, which may be sharp, in the handle may allow the handle and pullwire to be manipulated more safely, preventing injury to the surgeon or others from the distal tip.

In some variations, the method also includes the step of sliding the pullwire handle distally to choke up on the pullwire within the body of the pullwire handle device.

The method may also include the step of pushing a button to lock the pullwire within the handle.

In some variations, the step of inserting the distal end of the pullwire into the pullwire handle device may include inserting the distal end of the pullwire into the funnel-shaped proximal end of the pullwire handle device. The step of indicating that the pullwire has been secured within the pullwire handle device may include displaying a portion of the pullwire through a window on the pullwire handle device.

Described herein are pullwire handle devices for securing to the distal end of a tissue-penetrating pullwire that include a handle body, a pullwire lock configured to removably lock onto a portion of a pullwire within the handle, and a storage region configured to store a portion of the pullwire within the handle. In some embodiments, the device may further include a funnel-shaped pullwire feed region configured for inserting the distal end of a pullwire within the handle body. In some embodiments, the device may further include an internal track for guiding the movement of the pullwire within the handle body.

In some embodiments, the pullwire lock may comprise a cam surface coupled to a clamp configured to lock the pullwire within the handle. In some embodiments, the pullwire lock may comprise a button, coupled to a lever arm configured to inactivate the lock and the lever arm may include a cam surface configured to activate and inactivate the lock.

In some embodiments, the pullwire lock is configured to lock the pullwire such that the pullwire handle can be locked to the pullwire, and the handle can be pulled to pull the pullwire so that it transmits between 10 and 60 pounds without the handle slipping, while in some embodiments, the pullwire lock is configured to lock the pullwire such that the pullwire can transmit over 50 pounds without slipping relative to the handle.

In some embodiments, the storage region includes a cone surface that functions to guide the pullwire into the storage region such that the pullwire buckles and is wound within the storage region.

Also described herein are methods of capturing a pullwire using a pullwire handle device configured to secure to the distal region of a pullwire. In some embodiments the method includes the steps of inserting the distal end of a pullwire into the pullwire handle device, storing a portion of the pullwire within the pullwire handle device, and activating the pullwire lock to lock the pullwire within the handle.

In some embodiments, the step of inserting the distal end of the pullwire into the pullwire handle device comprises inserting the distal end of the pullwire into the funnel-shaped proximal end of the pullwire handle device. The method of securing the pullwire and the handle together may include a step of inactivating a pullwire lock configured to lock the pullwire within the handle, prior to the inserting step. In some embodiments, the activating step includes rotating a cam surface against a pullwire, wherein the cam is configured to lock the pullwire within the handle.

As mentioned, the activating step may further include activating the pullwire lock such that the locked pullwire can transmit between 10 and 60 pounds without slipping, while in some embodiments, the activating step further includes activating the pullwire lock such that the locked pullwire can transmit over 50 pounds without slipping.

Also described herein are methods of treating a patient using the handle devices for securing to the distal end of a tissue-penetrating pullwire.

Also described herein are pullwire handle devices for securing to a tissue-penetrating pullwire. In some embodiments, the device includes a handle body, a pullwire lock configured to removably lock the pullwire handle device onto a pullwire within the handle body, and a tip containment element configured to retain the distal tip of the pullwire. In some embodiments, the handle body further comprises a storage chamber configured to store a distal portion of the pullwire.

In some embodiments, a pullwire handle device for securing to a tissue-penetrating pullwire includes a pullwire storage chamber configured to store a distal portion of the pullwire, and an internal track for guiding the pullwire into the pullwire storage chamber. The pullwire handle device may be configured to removably lock the pullwire handle device onto a pullwire. In some embodiments, the handle body further includes a pullwire feed region configured to receive the distal portion of the pullwire.

In some embodiments, a pullwire handle device for securing to a tissue-penetrating pullwire includes a handle body, and a pullwire lock configured to removably lock the pullwire handle device to a pullwire. In some embodiments, the pullwire lock includes a clamp plate and a cam surface configured to apply a force to push the clamp plate to lock the pullwire within the pullwire handle device. In some embodiments, the handle body further comprises a storage chamber configured to store a distal portion of the pullwire.

In some embodiments, the storage chamber includes a cone surface that functions to guide the pullwire into the storage chamber such that the region of the pullwire within the handle buckles (or bends) and is stored within the storage chamber. In some embodiments, the storage chamber includes a slot that may be configured to allow a portion of the pullwire to pass through the slot and out of the storage chamber. In some embodiments, the device further includes a pullwire guide configured to store a portion of the pullwire. The pullwire guide may be configured to break-away from the handle body.

In some embodiments, the pullwire lock includes a lever arm and a button coupled to the lever arm, wherein the button and lever arm are configured to activate and inactivate the lock. The lever arm may include a cam surface configured to removably lock the pullwire handle device onto a pullwire. In some embodiments, the pullwire lock further includes a clamp plate. The cam surface may be configured to apply a force to push the clamp plate to lock the pullwire within the pullwire handle device. In some embodiments, as a portion of a pullwire moves out of the pullwire handle device, the cam surface is configured to apply an increased force to further push the clamp plate and lock the pullwire within the pullwire handle device. In some embodiments, the pullwire lock further includes a second clamp plate. In some embodiments, the second clamp plate may be fixed with respect to the pullwire lock. In some embodiments, the first and second clamp plates are configured to receive a pullwire between them such that an interior portion of the clamp plates couples to a pullwire. The cam surface may be coupled to an exterior portion of a clamp plate.

In some embodiments, the tip containment element includes a storage chamber configured to store a distal portion of the pullwire. The tip containment element may be configured to retain the distal tip of the pullwire within the handle body.

Also described herein are methods for capturing a pullwire using a pullwire handle device configured to secure to the distal end of a pullwire. In some embodiments, the method includes the steps of inserting the distal end of a pullwire into the pullwire handle device, advancing the pullwire further into the pullwire handle device while the distal portion of the pullwire is contained within the pullwire handle device, and locking the distal portion of the pullwire within the pullwire handle device. In some embodiments, the method further includes the step of storing a distal portion of the pullwire within the pullwire handle device. The inserting step may include inserting the distal end of the pullwire into a funnel-shaped proximal end of the pullwire handle device.

In some embodiments, the advancing step includes advancing the pullwire into a storage chamber of the pullwire handle device. The advancing step may further include advancing the pullwire against a surface within the storage chamber such that the pullwire buckles and is stored within the storage chamber.

In some embodiments, the locking step includes pressing a button coupled to a locking mechanism to lock the locking mechanism and lock the distal portion of the pullwire within the pullwire handle device. While in some embodiments, the locking step includes releasing a button coupled to a locking mechanism to lock the locking mechanism and lock the distal portion of the pullwire within the pullwire handle device. In some embodiments, the locking step includes moving the pullwire and pullwire handle device with respect to one another to lock the distal portion of the pullwire within the pullwire handle device. Moving the pullwire and pullwire handle device with respect (e.g., in opposite directions) to one another may include moving the pullwire and pullwire handle device with respect to one another such that a portion of the pullwire moves out of the pullwire handle, for example, pulling the handle distally or holding it still while pulling the pullwire proximally. Proximally and distally may refer to the axial directions of the pullwire.

In some embodiments, the method includes the steps of inserting the distal end of a pullwire into the pullwire handle device, moving a cam surface of a locking mechanism of the pullwire handle device such that the cam surface applies a force to a clamp plate and the clamp plate applies a force to the pullwire, and moving the clamp plate within the pullwire handle device such that the cam surface applies an increased force to the clamp plate and the clamp plate secures the pullwire. In some embodiments, the inserting step includes inserting the distal end of the pullwire into a funnel-shaped proximal end of the pullwire handle device.

In some embodiments, the moving a cam surface step includes rotating the cam surface against a clamp plate and moves the clamp plate toward the pullwire. In some embodiments, the moving the clamp plate step includes moving the clamp plate within the pullwire handle device such that the cam surface further rotates against a clamp plate and further moves the clamp plate toward the pullwire.

In some embodiments, the moving a cam surface step includes pressing a button coupled to a first end of a lever arm, wherein the second end of the lever arm comprises the cam surface. While in some embodiments, the moving a cam surface step includes releasing a pressed button coupled to a first end of a lever arm, wherein the second end of the lever arm comprises the cam surface. For example, as the button is released, the lever arm rotates, thereby rotating the cam surface against the clamp plate.

In some embodiments, the moving the clamp plate step comprises moving the pullwire and pullwire handle device with respect to one another such that the clamp plate moves within the pullwire handle device. Moving the pullwire and pullwire handle device with respect to one another may include moving the pullwire and pullwire handle device with respect to one another such that a portion of the pullwire moves out of the pullwire handle and the clamp plate moves toward the proximal end of the pullwire handle device.

In some embodiments, the method further includes the step of advancing the pullwire further into the pullwire handle device while the distal (tip or end) portion of the pullwire is contained within the pullwire handle device. The advancing step may include advancing the pullwire into a storage chamber of the pullwire handle device. In some embodiments, the advancing step further includes advancing the pullwire against a surface within the storage chamber such that the pullwire buckles and is stored within the storage chamber.

In some embodiments, the methods further include the step of pulling on the pullwire handle device to advance the pullwire in a distal direction. Pulling on the pullwire handle device may advance a proximal portion of the pullwire in a distal direction through a spinal foramen. In some embodiments, the pulling step includes pulling on the pullwire handle device to transmit a force to a proximal portion of the pullwire, wherein the force is greater than 10 pounds. In some embodiments, the pulling step includes pulling on the pullwire handle device to transmit a force to a proximal portion of the pullwire, wherein the force is greater than 35 pounds. In some embodiments, the method further includes the step of coupling a device to a proximal region of the pullwire. Pulling on the pullwire handle device may advance the device in a distal direction through a spinal foramen.

Any of the methods described herein may also include the step of coupling the proximal end region of the pullwire to an elongate device (e.g., a tissue modification device, neural localization device, etc.) either before or after attaching the pullwire handle. The elongate device may be attached end-to-end with the pullwire.

Any of the devices described herein (including the probe(s), tissue modification devices, neural localization devices, etc.) may include one or more safety or retainer features, including a safety retainer. In general, a safety retainer retains a portion of the device that break off or away from the device and prevents such portions from being left behind when the device is otherwise removed from the patient. For example, in some variations the safety retainer extends longitudinally along the length of the device. In some variations the safety retainer is an elongate member such as a wire, cord, band, fiber, or the like. The safety retainer may be loosely held within the device (in slack). Thus, in some variations the safety retainer is slideably held within the device or a portion of the device. In some variations the safety retainer is held slack within the device because the length of the safety retainer is longer than the length of the region within which it is held; alternatively or additionally the safety retainer may be slightly compressed along its length within the device.

In some variations the safety retainer is a continuous loop within the device, while in some variations it is not a loop. The safety retainer may be anchored (e.g., secured) at one or more location within the device, or it may be loosely held within the device without being anchored, although it may still be held securely within the device. For example, a safety retainer may be held within one or more channels passing through the device.

In some embodiments, a probe device for positioning a wire around a target tissue may include a rigid outer cannula having a curved distal region; a flexible inner cannula slideably disposed within the rigid outer cannula, wherein the inner cannula is configured to assume a curved shape when extended distally from the outer cannula; a safety retainer extending distally; and a distal tip at the end of the inner cannula, the distal tip coupled to the safety retainer. The safety retainer may be coupled to the distal tip in any appropriate manner to secure the distal tip should it (or a more proximal region of the inner cannula) break off from the rest of the device. For example, the safety retainer may loop through a region within the distal tip; in some variations the safety retainer is fixedly secured to the distal tip (or to other regions of the device), e.g., by crimping, adhesive, etc.

In some embodiments, the distal tip comprises a port for the exit of a wire through the distal tip. In some embodiments, the safety retainer is secured to the probe near the proximal end region of the safety retainer. In some embodiments, the safety retainer comprises a cable extending within a safety retainer lumen within a cannula wall of the inner cannula.

In some embodiments, the cable is slideably disposed within the safety retainer lumen within the cannula wall of the inner cannula. In some embodiments, the safety retainer comprises a loop of material extending within a first safety retainer lumen extending through the cannula wall, through the distal tip, and extending within a second safety retainer lumen extending through the cannula wall. In some embodiments, the loop of cable is slack within the safety retainer lumens of the inner cannula. In some embodiments, the safety retainer comprises a cable disposed within a central lumen of the inner cannula. In some embodiments, the cable is slideably disposed within the central lumen of the inner cannula. In some embodiments, the cable is slack within the central lumen of inner cannula. In some embodiments, the cable is Nitinol. In some embodiments, the safety retainer comprises a loop of material extending within a central lumen of the inner cannula, through the distal tip, and extending back within the central lumen of the inner cannula.

In some embodiments, the probe device further includes a longitudinally extending shape memory member. In some embodiments, the shape memory member is a tubular member disposed within a central lumen within a cannula wall of the inner cannula. In some embodiments, the shape memory member is a tubular member disposed within a central lumen of the inner cannula adjacent to the safety retainer also within the central lumen. In some embodiments, the shape memory member is a Nitinol tube.

In some embodiments, a probe device for positioning a wire around a target tissue may include a rigid outer cannula having a curved distal region; a flexible inner cannula slideably disposed within the rigid outer cannula, wherein the inner cannula is configured to assume a curved shape when extended distally from the outer cannula; a distal tip at the distal end of the inner cannula; and a safety retainer cable coupled to the distal tip and secured to the probe proximally from the distal end of the safety retainer; wherein the safety retainer cable extends proximally from the distal tip and is slack.

In some embodiments, a probe device for positioning a wire around a target tissue may include an outer cannula having a curved distal region and an inner member slideably disposed within the outer cannula. In some embodiments, the inner member includes an elongate body, a longitudinally extending support member disposed inside the elongate body, and a longitudinally extending tubular body disposed inside the elongate body, configured to assume a curved shape when extended distally from the outer cannula, and configured to pass a wire, and a distal tip coupled to the support member.

In some embodiments, the elongate body comprises a central lumen configured to receive the tubular body and at least one support member lumen to receive the support member. In some embodiments, he elongate body comprises a single central lumen and the support member and the tubular body are disposed inside the central lumen of the elongate body. In some embodiments, the solid support members are curved. In some embodiments, the solid support members comprises a shape memory alloy. In some embodiments, the tubular body is a shape memory material. In some embodiments, the tubular body is Nitinol. In some embodiments, the tubular body has a curved distal end. In some embodiments, the tubular body is flanked by solid support members.

In some embodiments, a probe device for positioning a wire around a target tissue may include an outer cannula having a curved distal region sized and configured to be advanced towards a lateral recess and a neural foramen in a patient; an inner cannula slideably disposed within the outer cannula, wherein the inner cannula is configured to assume a curved shape when extended distally from the outer cannula and configured to extend through the neural foramen and at least partially around an anterior portion of a facet joint and posterior to a spinal disc; and a proximal handle comprising a hand grip region coupled to the outer cannula and a plunger region coupled to the inner cannula.

In some embodiments, a probe device for positioning a wire around a target tissue may include an outer cannula having a curved distal region; an inner cannula slideably disposed within the outer cannula, wherein the inner cannula is configured to assume a curved shape when extended distally from the outer cannula; a proximal handle comprising a hand grip region coupled to the outer cannula and a plunger region coupled to the inner cannula; wherein the proximal end of the plunger region comprises a funneled port for insertion of a wire through the probe.

In some embodiments, a probe device for positioning a wire around a target tissue may include a straight outer cannula; an inner cannula slideably disposed within the outer cannula, wherein the distal end region of the inner cannula is curved and extends distally from the outer cannula; a flexible catheter slideably disposed within the outer cannula and the inner cannula, wherein the flexible catheter is configured to assume a curved shape when extended distally from the outer cannula; and a proximal handle comprising a hand grip region coupled to the outer cannula and a plunger region coupled to at least one of the inner cannula and the flexible catheter; wherein the proximal end of the handle comprises a funneled port for insertion of a wire through the probe.

In some embodiments, the inner cannula is rigid. In some embodiments, the inner cannula is flexible. In some embodiments, the inner cannula is a shape memory material and is configured to assume a curved shape when extended distally from the outer cannula. In some embodiments, the proximal handle comprises a first plunger coupled to the inner cannula and a second plunger coupled to the flexible catheter. In some embodiments, the second plunger comprises the funneled port for insertion of a wire through the probe. In some embodiments, the second plunger is slideably disposed within the first plunger.

In some embodiments, a probe device for positioning a wire around a target tissue may include a rigid outer cannula having a curved distal region; a flexible inner cannula slideably disposed within the rigid outer cannula, wherein the inner cannula is configured to assume a curved shape when extended distally from the outer cannula; a neural localization member configured to be deployed over the flexible inner cannula. In some embodiments, the neural localization member includes a flexible, elongate, ribbon-shaped body extending distally, wherein the body comprises a front side and a back side, and a first electrode coupled to the front side of the elongate, ribbon-shaped body and configured to stimulate adjacent nerve tissue. In some embodiments, the probe device further includes a proximal handle comprising a hand grip region coupled to the outer cannula and a plunger region coupled to at least one of the inner cannula and the neural localization member; wherein the proximal end of the handle comprises a funneled port for insertion of a wire through the probe.

In some embodiments, the probe device further includes a second electrode coupled to the back side of the elongate, ribbon-shaped body and configured to stimulate adjacent nerve tissue. In some embodiments, the neural localization member further comprises a track that couples to at least one of the outer cannula and the inner cannula such that the neural localization member is deployable over the flexible inner cannula. In some embodiments, the proximal handle comprises a first plunger coupled to the inner cannula and a second plunger coupled to the neural localization member. In some embodiments, the first plunger comprises the funneled port for insertion of a wire through the probe. In some embodiments, the second plunger is slideably disposed within the first plunger.

In some embodiments, probe device for positioning a wire around a target tissue may include a rigid outer cannula having a curved distal region; a flexible inner cannula slideably disposed within the rigid outer cannula, wherein the inner cannula is configured to assume a curved shape when extended distally from the outer cannula; a flexible guidewire slideably disposed within the flexible inner cannula; and a proximal handle comprising a hand grip region coupled to the outer cannula and a plunger region coupled to at least one of the inner cannula and the guidewire.

In some embodiments, the proximal handle comprises a first plunger coupled to the inner cannula and a second plunger coupled to the guidewire. In some embodiments, the second plunger is slideably disposed within the first plunger. In some embodiments, the first plunger is slideably disposed within the second plunger. In some embodiments, the flexible inner cannula is configured to extend distally from the outer cannula over the guidewire.

In some embodiments, a method of accessing a spine of a patient may include the steps of inserting a first probe instrument between an interlaminar window and toward a spinal disc of a patient; advancing the distal end of the probe instrument into a neural foramen onto the spinal disc; pushing the spinal disc material down with the distal end of the probe instrument in an anterior direction; and rotating the distal end of the probe instrument against a first and second pedicle of the neural foramen such that the first and second pedicles are distracted from one another.

In some embodiments, the step of locking the distal end of the probe instrument in position. In some embodiments, the method may further include the step of advancing a second probe instrument between the distracted first and second pedicle. In some embodiments, the method may further include the step of advancing a wire through the second probe instrument between the distracted first and second pedicle and exiting the patient. In some embodiments, the method may further include the step of coupling a tissue modification device to the wire and pulling the tissue modification device between the distracted first and second pedicle with the wire.

In some embodiments, a probe device for positioning a wire around a target tissue may include a rigid outer cannula having a curved distal region; a flexible inner cannula slideably disposed within the rigid outer cannula, wherein the inner cannula is configured to assume a curved shape when extended distally from the outer cannula. In some embodiments, the flexible inner cannula may include an elongate body comprising a working channel and at least one support member lumen, and at least one support member, wherein the support member is configured to assume a curved shape when extended distally from the outer cannula.

In some embodiments, the elongate body comprises a working channel disposed between a first support member lumen and a second support member lumen. In some embodiments, the support member is a nitinol wire. In some embodiments, the elongate body is tapered at the distal end.

In some embodiments, a probe device for removing tissue may include an elongate rigid member having a curved distal end sized and configured to be advanced towards a lateral recess and a neural foramen in a patient, wherein the curved distal end is sized and configured to extend posterior to a spinal disc through the neural foramen and at least partially towards an anterior portion of a facet joint; and a cutting member at the distal end of the rigid member, wherein the cutting member is a scooped cutting member with at least one bladed edge.

In some embodiments, the elongate member further comprises a channel disposed along the length of the member. In some embodiments, the channel is sized and configured to receive a guidewire. In some embodiments, the channel is sized and configured for suction and irrigation. In some embodiments, the channel is sized and configured to receive a guidewire and the member further comprises at least a second channel for suction and irrigation. In some embodiments, the cutting member is configured to cut both soft tissue and bone. In some embodiments, the cutting member is configured to cut a portion of the anterior portion of the facet joint. In some embodiments, the device may further include an electrode toward the distal end of the rigid member configured to stimulate adjacent nerve tissue.

### BRIEF DESCRIPTION OF THE DRAWINGS

FIGS. 1A-1E illustrates a system including tools for treating spinal stenosis, including a pullwire, a removable pullwire handle, a tissue modification tool, a flexible neural localization tool, and a pullwire positioning probe tool.

FIG. 2A shows a side perspective view of an alternative view of the proximal end of another variation of a probe.

FIG. 2B illustrates a perspective view of a probe as in FIG. 2A when handheld.

FIGS. 3A-3N illustrate alternative variations of probes that may be used to position a pullwire, as described herein.

FIGS. 4A and 4B show a front and two side perspective views, respectively, of a handle of a probe such as the one shown in FIGS. 2A and 2B.

FIG. 4C shows a top perspective view of the proximal end of the same guide.

FIGS. 5A-5C illustrate methods of manipulating the probe shown in FIGS. 4A-4C.

FIGS. 6A and 6B show an exposed view of the internal workings of another variation of a probe device.

FIG. 7A shows a partial cross-section through the distal region of one variation of a probe, and FIG. 7B shows a partial cross-section through a more proximal region of the probe.

FIGS. 7C and 7D show another variation of the distal region of a probe including a tip capture mechanism.

FIG. 7E shows a more proximal region of the probe shown in FIGS. 7C and 7D.

FIGS. 7F and 7G show alternative configurations for probes including tip capture mechanisms such as those of FIGS. 7A-7C.

FIG. 7H illustrates one variation of a probe including a stylet such as those shown in FIGS. 7A-7C.

FIGS. 7I and 7J illustrate another two variations of the distal end regions of probes that may be used to position a pullwire around a target tissue.

FIGS. 7K and 7L illustrate two variations of the distal end regions of probes similar to those shown in FIGS. 7C and 7D, indicating exemplary dimensions.

FIGS. 7M and 7N show different variations of the distal tip region of a probe including a safety retainer.

FIGS. 8A and 8B shows side perspective views of two variation of neural localization devices (“NLR devices”) as described herein.

FIGS. 9A and 9B show front and side views, respectively, of the proximal (handle) portion of a neural localization device.

FIGS. 10A, 10B and 10C show alternative views of the proximal (e.g., handle) portion of the neural localization device shown in FIGS. 9A and 9B.

FIGS. 11A and 11B show front and side perspective views, respectively, of the proximal end region (e.g., the handle) of a tissue modification device.

FIGS. 12A, 12B and 12C show alternative side and end views of the proximal end of the tissue modification device shown in FIGS. 11A and 11B.

FIGS. 13A and 13B illustrate method of holding or manipulating the tissue modification devices similar to those shown in FIGS. 11A-12C.

FIGS. 13C and 13D illustrate alternative views of a portion of a tissue modification device that may be used with the handles shown in FIGS. 11A-13B.

FIGS. 14A and 14B illustrate side and top views, respectively, of one variation of a handle that may be removably attached to a pullwire, as described herein. FIG. 15D is a side perspective view of another variation of a handle that may be used with a pullwire.

FIGS. 15A-15D illustrate different variations of a handle that may be removably attached to a pullwire.

FIGS. 16A and 16B show front and side views, respectively of a handle such as the one shown in FIG. 15D.

FIGS. 17A, 17B and 17C show alternative side views of the proximal end of a handle for use with a pullwire such as the handle shown in FIGS. 16A-16B.

FIGS. 18A-18E show an alternative distal pullwire handle. FIGS. 18A and 18B show side and front perspective views, respectively, of this variation, while FIGS. 18C and 18D show partially transparent side and perspective views, respectively. FIG. 18E shows a cross-section through the handle shown in FIGS. 18A and 18B.

FIGS. 19A-19K show a distal pullwire handle, similar to the variation of FIGS. 21A-21E. FIG. 19A shows an exploded view of this variation. FIG. 19B shows a cross sectional view, and FIG. 19C shows a partially exploded view of this variation. FIG. 19D shows an exploded view of the lock mechanism of the pullwire handle. FIG. 19E shows a partial cross-section through the handle and FIG. 19F shows a detail view of the section of FIG. 19E. FIGS. 19G and 19H show a side and cross sectional view respectively, of a pullwire guide. FIGS. 19I-19K show a storage region of the pullwire handle.

FIGS. 20-24C show various alternative lock mechanisms of a distal pullwire handle.

FIGS. 25A-25B show perspective views of another variation of a handle for the distal end of a pullwire. This variation includes a passive loop configuration for managing the distal end of the pullwire.

FIG. 26A shows another variation of a pullwire handle similar to that shown in FIGS. 25A and 25B.

FIGS. 26B-26G illustrate the operation of the distal handle shown in FIG. 26A.

FIG. 27A shows a perspective view of another variation of a pullwire handle. FIGS. 27B-27D illustrate operation of the controller for the pullwire handle. FIGS. 27E and 27F show side and cross-sectional views, respectively, of the handle of FIG. 27A during a first operational state; and FIGS. 27G and 27H show side and cross-sectional views, respectively, of the handle of FIG. 27A during a third operational state.

FIGS. 28A-28F illustrate another variation of a system with tools for treating spinal stenosis including two variations of a pullwire positioning probe tool (28A and 28B), a flexible neural localization tool (28C), a tissue modification tool (28D), a removable pullwire handle (28E), and a pullwire (28F).

FIG. 29A is another variation of a probe as described herein.

FIG. 29B shows a cross-section through the distal end region of the probe of FIG. 29A.

FIGS. 30A-30I illustrate alternative views of one variation of the distal end of a probe such as the probe shown in FIG. 29A.

FIGS. 31A-31I illustrate alternative views of another variation of the distal end of a probe such as the probe shown in FIG. 29A.

FIGS. 32A-32I show the alternative distal end views shown in FIGS. 31A-I including the support wire.

FIGS. 33A-33C show various cross-sections through the distal end region of the probe, specifically the inner cannula.

FIG. 34 shows a variation of the distal end of a probe such as the probe shown in FIGS. 33A-33C.

FIG. 35 shows a probe with a stiffening tube.

FIG. 36 is a posterior view of the spine indicating decompression paths at disk level and along the nerve root.

FIG. 37 is a posterior view of the spine indicating a decompression path for adjacent level lateral recess decompression.

FIGS. 38 and 39 illustrate a probe for positioning a guidewire.

FIGS. 40A-40C illustrate an alternative probe configuration for positioning a guidewire.

FIGS. 41A-41C illustrate various probe configurations.

FIG. 42 illustrates, in an axial view, a probe positioned within a spine of a patient.

FIGS. 43A-43C illustrate various distal tip configurations.

FIGS. 44 and 45 illustrate access devices and probes.

FIGS. 46, 47, 48, 49, 50, 51 and 52 illustrate alternative variations of an access device configured to aid in the removal of the device.

FIGS. 53A-53B, 54A-54B, 55, 56, 57 and 58A-58B illustrate alternative variations of an access device configured to aid in accessing the spine with the access device.

FIGS. 59A-59B, 60, 61, 62, 63, 64A-64D, 65A-65B, 66 and 67 show various embodiments of instruments and probes, such as access probes.

FIGS. 68, 69 and 70A-70B illustrate various embodiments of an access probe with or without neural localization capabilities.

### DETAILED DESCRIPTION

The devices, systems and methods described herein may be used in any appropriate surgical procedure, particularly for the surgical treatment of spinal stenosis. For example, described herein are systems including one or more of the following devices: a pullwire, a handle for the distal end of a pullwire, a probe for positioning a pullwire, a neural localization device for use with a pullwire and a tissue modification device for use with the pullwire.

In particular, described herein are devices and systems including these devices that are configured for use together as a system. For example, the devices described herein may all be coordinated so that they may function together, and may include markings, orienting structures and other features that are common between the different devices within the system. In some variations the devices all include front/back, top/bottom, or other orientation structures on the handles of the devices. The handles may be structured in common.

The devices described herein may include handles that allow the devices to be hand operated using one hand or two hands (or both). In some variations, the devices include handles that guide or regulate the hand position when the device is in use. For example, the devices may regulate hand position. Coordinating or regulating hand position may be particularly important during surgical procedures using these devices to access difficult to reach and/or otherwise sensitive regions of a patient's body.

Many of the devices described herein may be used with a pullwire for either or both positioning of the device and operation of the device. Thus, many of the devices include pullwire management features to help position, orient, grasp, and regulate the pullwire. If the pullwire is not properly managed, it may prevent correct operation of the device, may increase risk of misuse of the device (potentially harming the patient), and may risk harming the operator (e.g., surgeon or other medical professional).

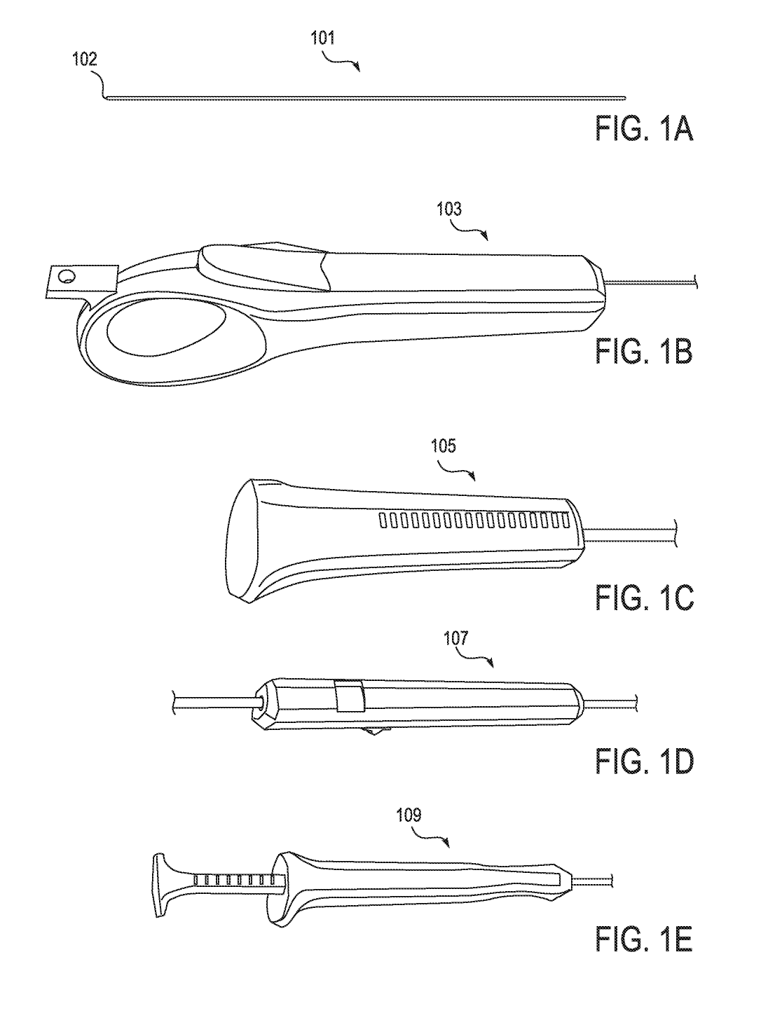

FIG. 1 shows one variation of a system for treating spinal stenosis. In FIG. 1, the proximal ends of a pullwire 101, a pullwire handle 103, a tissue modification device 105 (that may be used with a pullwire), a neural localization device 107 (that may be used with a pullwire) and a probe 109 that may be used to place a pullwire are all shown. In this example, as shown, the proximal regions of these devices are coordinated so that they share common features which facilitate their operation as a system. For example, the handles of these devices are all oriented with a flat ‘front’ region and a curved or projecting ‘back’ region. The flat front region is indicated by a different color, and may be textured so that it is apparent by the feel of the device that it is the front region. The flat front region may allow the devices to be used more effectively in an open, partially open, or minimally invasive procedure. As described below, when the devices are used to operate on spinal tissue, or other tissue that is otherwise difficult to access, the flat front may allow the visualization of the region of the target tissue even when the device is being held or manipulated.

The pullwire shown in FIG. 1 is typically long (e.g., elongated) and flexible, and may have a sharp (tissue penetrating) distal end, not shown, and a proximal end 102 that allows it to be coupled to a pullwire coupling member securely. For example, the pullwire may include a ball or other shaped end (which may be conical, tubular, ring, etc.) for coupling to a pullwire coupling member of a device (such as a tissue modification device or a neural localization probe). The proximal end 102 may be configured to lock into a pullwire coupling member at the distal end of a tissue modification device (such as the one 105 indicated in FIG. 1), and/or a neural localization device 107. Similarly, the proximal end of the pullwire may be configured to pass through the probe 109 so that the probe may be removed from over the proximal end of the pullwire during operation.

Each of these devices (the probe 109, the neural localization device 107, the tissue modification device 105 and the pullwire handle 103) are described in greater detail below.

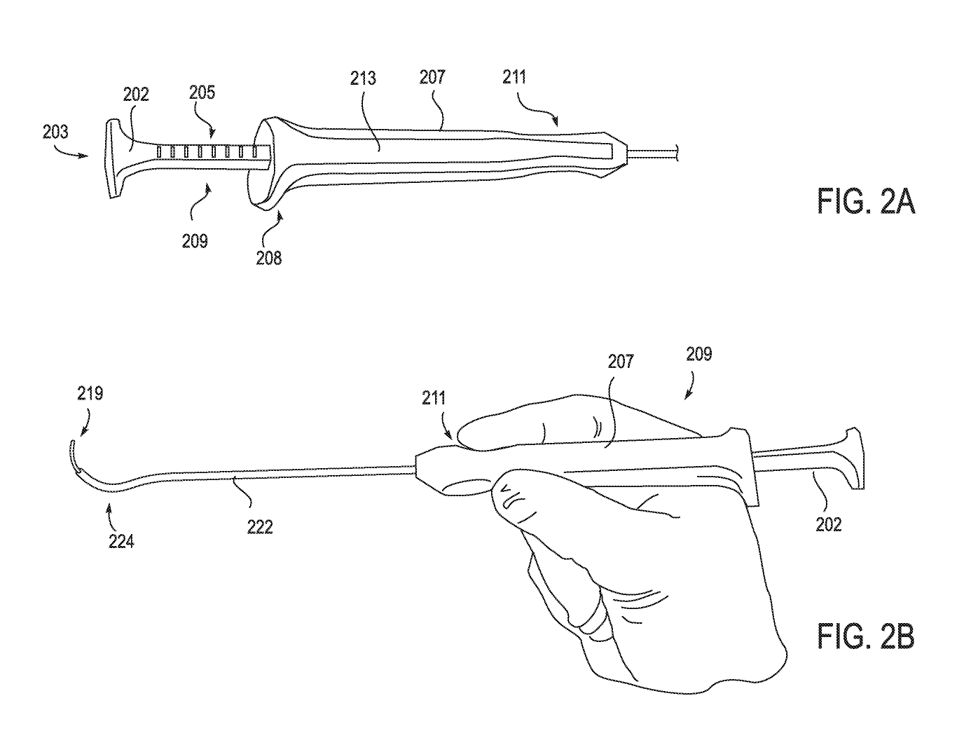

FIGS. 2A-7L describe and illustrate different variations of probes that may be used to position a pullwire. For example, FIG. 2A illustrates one variation of an improved probe having additional features including a calibrated, keyed, and flanged pusher 202.

For example, in the variation illustrated in FIGS. 2A-2B, the probe 209 includes a handle portion having a pusher 202 that communicates with an internal cannula slideably disposed within the external cannula so that the internal cannula may be extended from the distal end of the probe for placement around a target tissue, as illustrated in FIG. 2B. The pusher includes a flanged 203 proximal end having a finger (e.g., thumb) pushing surface that is perpendicular to the long axis of the device (including the long axis of the handle). As described in greater detail, this proximal end may be formed to more readily allow insertion of a pullwire by guiding the pullwire into the lumen of the inner cannula. The pusher is calibrated 205 along the side in a top-facing surface. The calibration shown in this example includes markings to indicate depth (e.g., how far down the pusher has been extended), which corresponds to how far out of the distal end the inner cannula is extended. The calibrations may include alphanumeric symbols, colors, textures, or any combination of the like. The calibrations may be referenced to distance (e.g., depth, length, etc.), or they may be un-referenced (as shown in FIG. 2A).

The handle portion of the probe 207 is configured to be readily gripped by a person's hand. The handle portion includes a front surface 213 that is substantially flat, and has a different feel (by the flat cross-section or texture, for example) than the other circumferential regions of the handle, which may be curved or rounded. In this variation, the flat surface not only provides tactile feedback to the user that this is the front of the probe (for orientation), but may also allow the user to visualize down the front length of the device for ease in viewing during operation. Thus, a user may be able to look down the front of the device, which typically corresponds to the direction of extension of the distal end of the inner cannula of the probe, as the probe is inserted into the tissue. This feature may be specifically useful when the probe is inserted into tissue via a minimally invasive and/or “mini-open” technique, such as through a tube. In this instance, the flat surface of the handle and the inner diameter of the tube may not contact one another, and may define a space through which a user may visualize into and/or through the tube and/or visualize tissue.

The handle portion 207 of the probe 209 may also include functional shapes for aiding in operation of the probe. For example, the proximal end of the handle region 207 may include a flange, lip, or rim 208 extending away from the front of the device, which does not extend towards the front (and otherwise block the view down the long front of the probe). This proximal rim may help provide leverage for operation of the probe, particularly when the pusher 202 is being driven to extend the inner cannula out of the distal end of the probe. In addition, the probe handle 207 may also include a waist region 211, that may also be referred to as a penciled region. This waist region 211 may be located distally along the handle, and may allow the device to be held pencil-like at the more distal end of the handle. For example, this region may be used to hold the probe using just the finger tips of the hand, improving the control and precision of operation, particularly during placement. This is illustrated in FIG. 2B.

In FIG. 2B, the probe 209 is held, pencil-like, with the fingertips grasping the waist region. The pusher 202 is shown partially pushed in, showing the distal end of the probe 219 extending partially from the distal end of the outer cannula 222. The outer cannula of the probe is fixed relative to the handle region 207, and had a curved distal end 224. In this example, the curve is crescent-shaped, so that it initially curves away from the centerline of the probe (e.g., towards the ‘back’ of the probe), and then curves back towards the front of probe, as shown. The inner cannula 219 of the probe is pre-biased so that it assumes a curved shape upon leaving the outer cannula 222. For example, the inner cannula may be a Nitinol cannula having a pre-set curved shape. The cannula may be solid, woven, mesh, etc., but includes a passageway for the pullwire. The distal tip of the inner cannula may be configured so that is substantially atraumatic. It may also be configured so that it cannot be withdrawn into the cannula (e.g., it may have a slightly larger OD than the ID of the outer cannula, etc.). In some variations, the distal tip may be blunted or rounded. Alternatively, the distal tip may be configured to cut tissue.

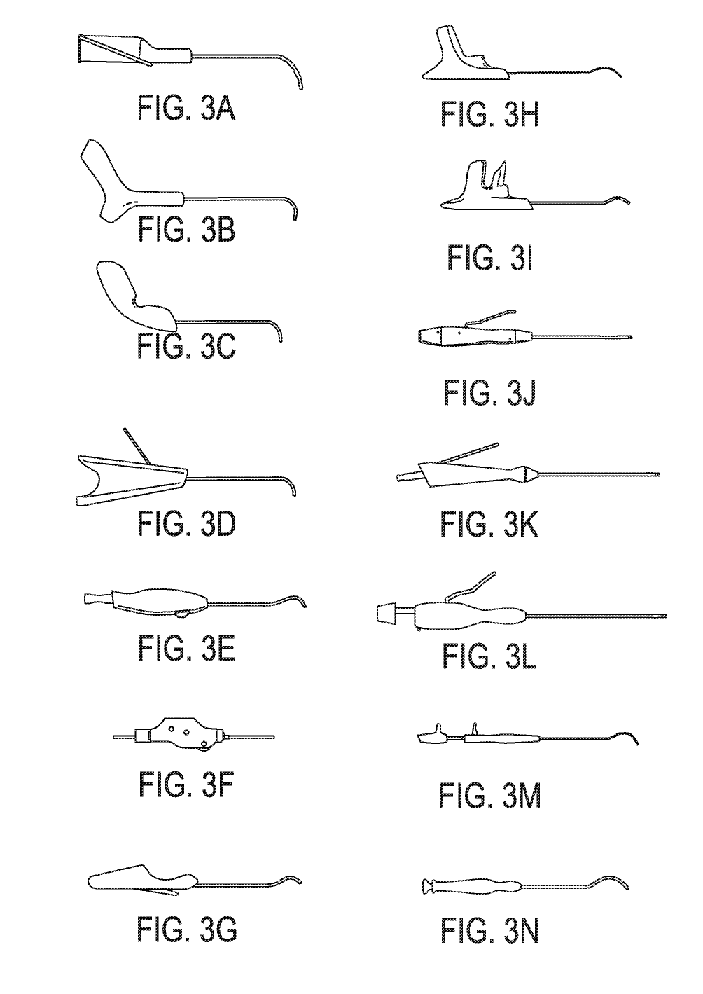

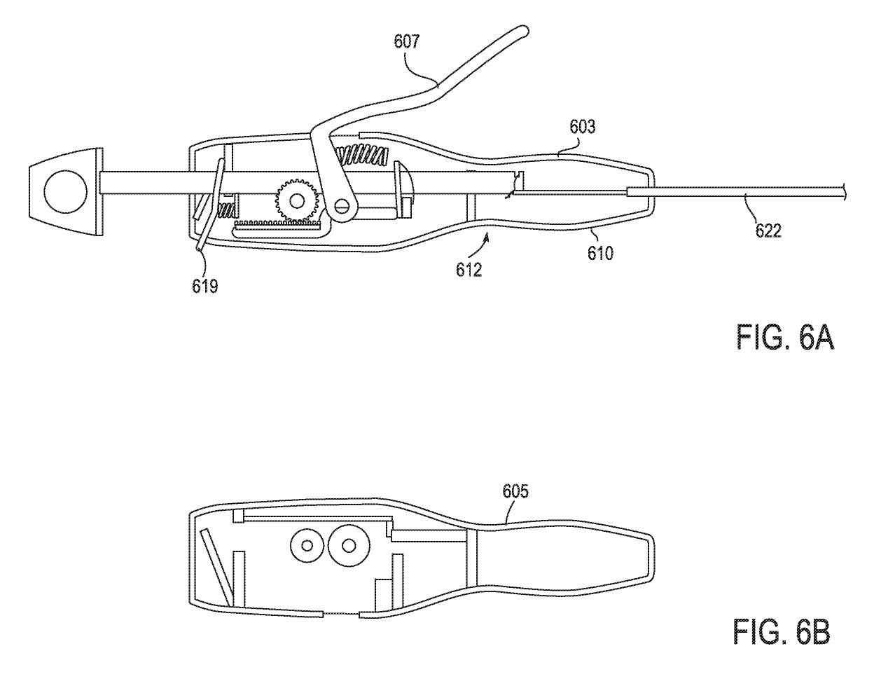

FIGS. 3A-3N illustrate alternative variations of the probe, including alternative variations of the handle. In some of these variations, for example, 3D, 3E, and 3G-3K, the handle portion of the probe includes a lever or switch portion that may be used to extend, lock, unlock, and/or retract the inner cannula of the probe. For example, the probe may include a switch or control for releasing the inner cannula so that it can be moved to extend or retract from the distal end. Thus, the probe may be locked, preventing motion of the inter cannula. FIGS. 6A and 6B illustrate another variation of a probe having a handle or control for regulating the motion of the inner cannula.

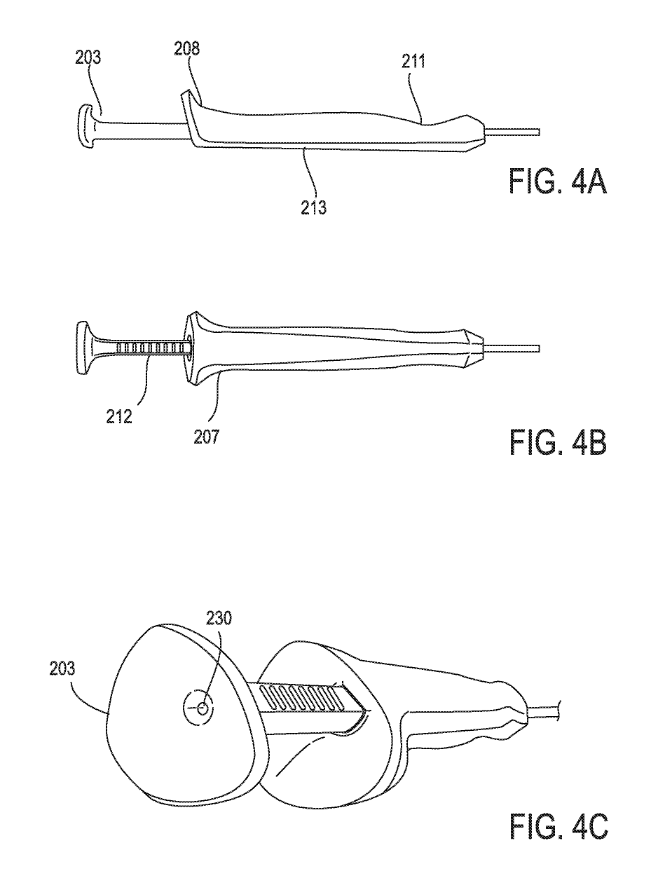

FIGS. 4A and 4B show front and side views, respectively, of the variation shown in FIG. 2A. FIG. 4C shows a top perspective view of the same variation. As mentioned above, the proximal end of the pusher 202 may be flanged. In some variations, as shown in FIG. 4C, the proximal end of the pusher (and thus the proximal end of the probe) may include a funnel-like structure 230 that is continuous with the inner channel through the cannula of the probe, to make insertion of the distal end of the (often sharp) pullwire into the probe both safer and simpler. Thus, the top end may for a convex or conical (e.g., funnel) shape for guiding the distal end of the pullwire through the inner cannula of the probe.

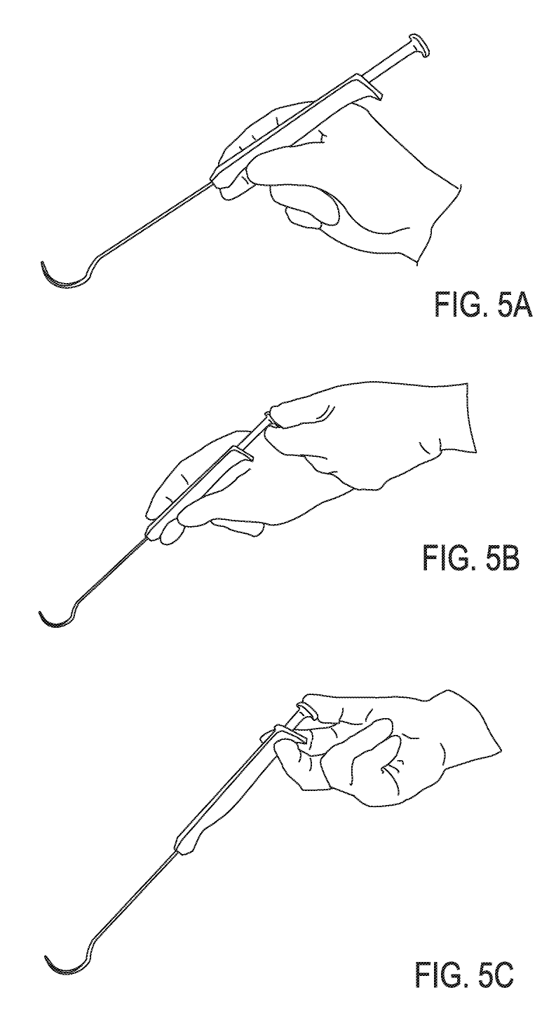

The variations of the probe shown above may be configured to operate in a two-handed or one-handed manner. FIGS. 5A-5C illustrate different methods of operating the probes described above, similar to FIG. 2B. FIG. 5A shows another example of holding the probe by the waist region near the distal end of the handle in a pencil-like grip. This may be referred to as penciling the probe. Thus, the waist region may be relatively narrow, and may include a concavity around the perimeter of the handle region into which the fingertips may fit. This narrowed region may allow the distal end of the probe to be manipulated with precision, allowing tilting, rotation and angling of the distal end. In this variation, the inner cannula may be operated by moving the pusher with the thumb, while penciling the probe with the fingers (for example, with the index and middle fingers). In FIG. 5B, the probe may be used in a two-handed arrangement. In this example, the probe is gripped in the right hand in a penciling grip, while the left hand is used to operate the inner cannula by moving the pusher. The flanged regions at the proximal end of the handle 208 and the pusher 203 provide leverage for both pushing and pulling. FIG. 5C illustrates one-handed deployment of the distal end of the probe (the inner cannula) using only one hand. In this operation, the proximal flange regions may be used as grips for the fingers, as shown.

FIGS. 6A and 6B illustrate another variation of a probe in which the handle portion is opened showing two housing portions 603, 605, and exposing the operation of the control (lever 607). In this variation, the control 607 may be any handle, lever, button, or the like. The control is geared to advance the inner cannula 610, as shown. As illustrated here, in some variations the pullwire may be fed through a side of the probe 612, rather than the proximal end of the probe, as shown in FIGS. 5A-5C. The variation shown in FIGS. 6A and 6B may also include a lock 619, which to prevent advancing and/or withdrawal of the inner cannula relative to the fixed outer cannula 622.

As mentioned, the probe inner cannula may be a shape-memory material or other material that assumes a straight or curved shape upon exiting the distal end of the fixed outer cannula. In some variations, the probe may include a safety retainer to retain the distal end of the inner cannula in the event of failure of the inner cannula during operation. In some variations, the safety retainer may prevent failure of the inner cannula. In this context, failure refers to the breaking off or bending of the inner cannula. For example, an inner cannula may break off during operation of a probe. This breakage may occur because of material fatigue, or because the probe must operate within bony or ligamentous regions that place stresses upon the distal tip regions of the probe, and particularly the inner cannula. The safety retainer may be used to capture or hold onto the distal tip should the inner cannula break.

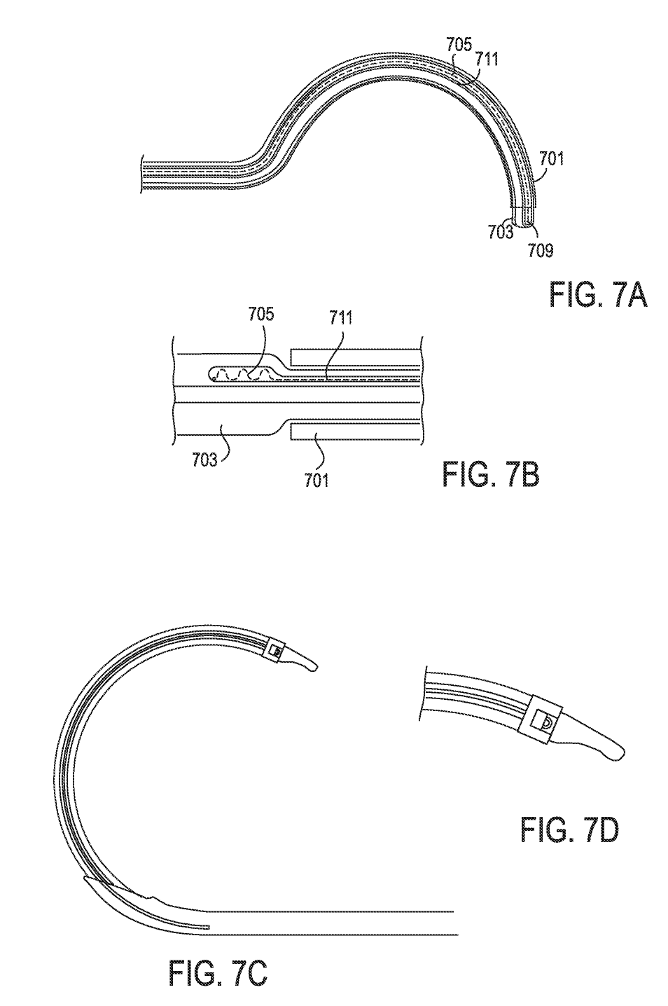

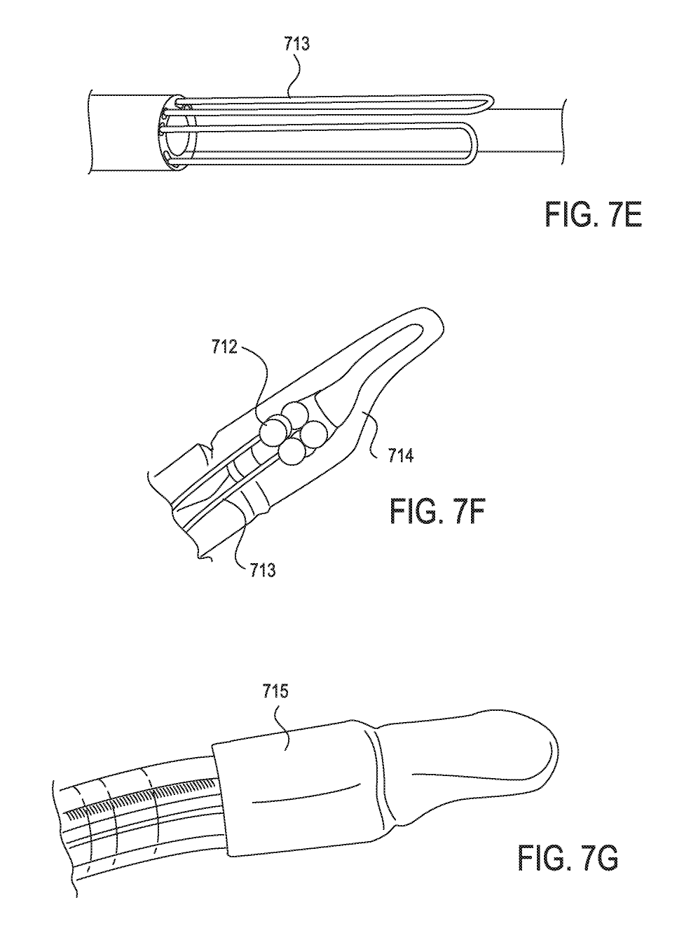

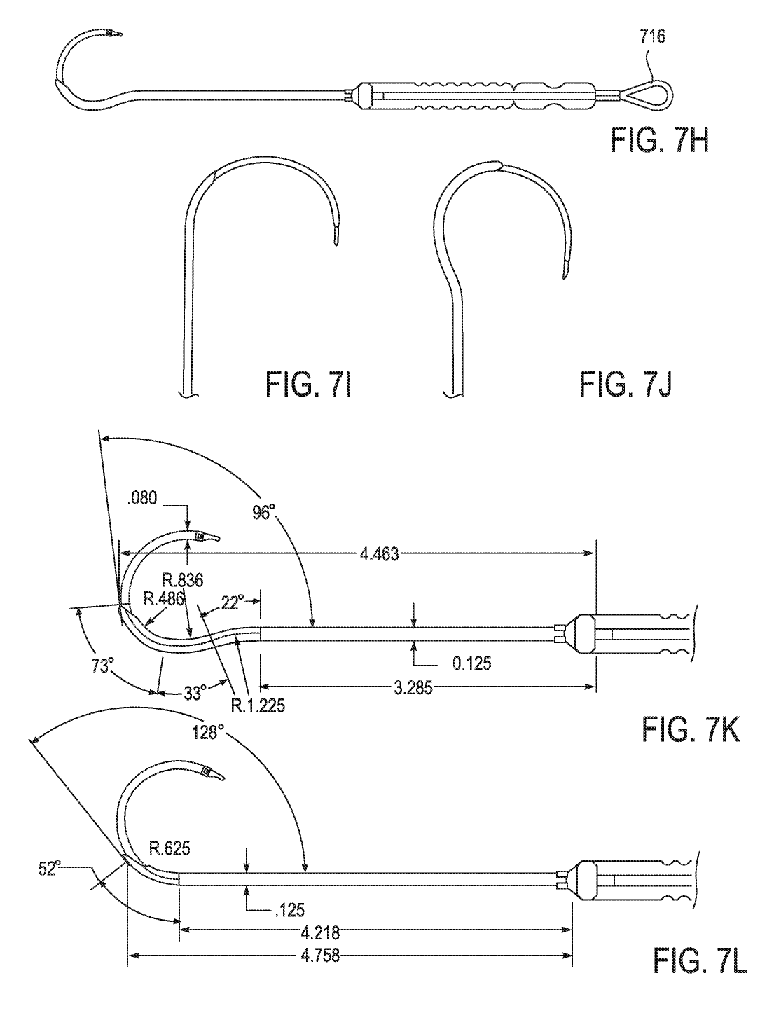

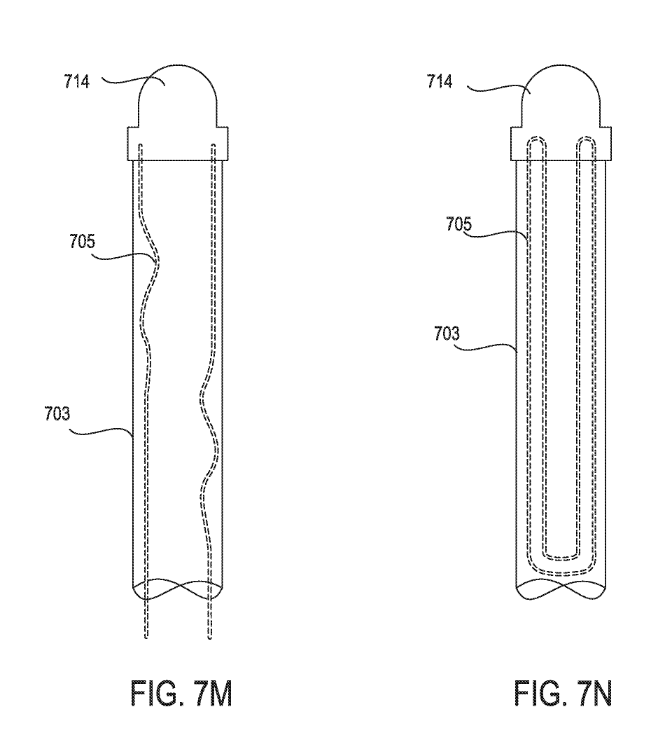

A safety retainer may include a tether, leash or the like that operates when the inner or outer cannula fail. For example, FIGS. 7A and 7B illustrates one variation of a safety retainer comprising a tether 705 that is secured 709 to the distal end of the inner cannula 703. In this variation, the tether is freely movable within an inner lumen or passage 711 within the inner cannula 703. Thus, as the inner cannula extends and bends, the tether (safety retainer 705) does not constrict the movement. The safety retainer may be coupled to the probe and to the distal tip such that the safety retainer remains slack within the probe. For example, the length of the safety retainer may be longer than the portion of the probe through which the safety retainer runs. FIG. 7A shows the distal end of one variation of a probe in cross-section. The inner cannula 703 is slideably movable within the outer (fixed) cannula 701, shown as transparent in this example. The inner cannula has a thickness that includes an inner, central, lumen that is configured for passage of a pullwire, and a peripheral side lumen 711 in which the safety retainer (tether 705) is located. As illustrated in FIG. 7B, the tether 705 is slack within the tether passage 711; in FIG. 7B, the proximal end of the tether includes additional material present in slightly expanded proximal end of the passage 711. In some variations the tether may be an elastomeric or other extendable material. In general the tether may be made of any appropriate material, and may be solid, woven, or the like. As shown in FIGS. 7M and N, the safety retainer 705 is slack within the inner cannula 703. As shown in FIG. 7M, a safety retainer cable 705 is coupled to the distal tip 714 and secured to the probe proximally from the distal end of the safety retainer. As shown, there is excess length of the safety retainer within the inner cannula. As shown in FIG. 7N, a safety retainer cable 705 is coupled to the distal tip 714 and secured to the probe proximally from the distal end of the safety retainer. As shown, there is excess length of the safety retainer within the inner cannula and it is wrapped back toward the distal end of the probe within the opposite peripheral side lumen.

In use, the safety retainer may act to prevent the distal end and/or the distal tip of the probe (and particularly the distal end of the inner cannula) from being left behind within the patient if it should break. The safety retainers described herein may also help retain the outer cannula in the event that it fails.

FIGS. 7C and 7D illustrate another variation of the distal end of a probe including a safety retainer, similar to the variation shown in FIGS. 7A and 7B and 7N. In this variation, the safety retainer is a loop of material that passes down the length of the inner cannula (within two lumen formed in the wall of the inner cannula. The material (e.g., a cable or wire of Nitinol, stainless steel, etc.) extends distally within a first lumen, then curves around at the distal end then extends proximally within a second lumen. The distal end (the loop) may be secured to the tip. For example, in FIG. 7C, the outer cannula is a shaped stainless steel cannula, and the inner cannula (“catheter”) is formed from multi-lumen polymer such as PEBAX or PEEK that is extruded over a shaped Nitinol hypotube. The safety retainer includes a Nitinol wire or cable that extends within the lumen formed in the wall of the polymeric extrusion (the “multi-lumen” extrusion). For example, a 0.006″ Nitinol wire with an outer polyimide cover (e.g., extruded on it) may run through the lumen in the polymeric sheath. The distal tip region of this example is shown in greater detail in FIG. 7D.

In FIG. 7D, the loop formed by the cable of the safety retainer passes through side flanges at the tip. The (atraumatic) tip is staked to the Nitinol hypotube. The cable can be secured to the tip by extruding material (e.g. PEBAX) over it, or by crimping, welding, or the like. For example, FIGS. 7F and 7G illustrate capture of the distal end of the safety retainer (e.g., cable) at the tip. In FIG. 7F (cross-sectional view), the cable 713 is captured by welding 712 the cable to the tip 714. In this illustration, the cable of the safety retainer is stainless steel. In FIG. 7G the safety retainer is welded to the tip which includes a skirt region 715. In some variations, this skirt may protect the tip and support the safety retainer. In some variations, the skirt may be crimped.

FIG. 7E illustrates the proximal end of the safety retainer within the inner cannula (shown in partial cut-away). In this example, the cables or wires 713 of the safety retainer are secured at the distal end. For example, the safety retainer material (e.g., wire or cable) may be fused to the inner cannula or catheter by fusing material, such as PEBAX, over them after they exit the lumen in the wall of the inner cannula, and after removing any cover on the wire or cable (e.g., a polyimide extrusion). In other variations the proximal end may be free and/or may exit the probe, as illustrated in FIG. 7H.

In FIG. 7H, the safety retainer comprises a cable running the length of inner cannula within one or more (in the case of looped or multiple strands of safety retainers) lumen in the wall of the inner cannula. The safety retainer may be secured to the proximal end, or it may exit (e.g., as a loop 716) the proximal end of the probe, as shown. As mentioned, in the event of a fracture of the inner cannula of the probe, the safety retainer keeps it connected. In some variations, the safety retainer may prevent the cannula from folding and/or flattening which may precede a complete detachment or fracture of the hypotube forming the cannula.

In general, the distal end of the probes described herein may be curved or bent, and/or may be curvable or bendable. As illustrated in the two variations shown in FIGS. 7I and 7J, the outer distal end may be more or less curved. The inner cannula may be configured to bend as it exits the distal end of the outer cannula, as shown, thereby increasing the ability of the probe to guide a pullwire around a target tissue.

FIGS. 7K and 7L illustrate two particularly useful variations of the probe ends that may be used. For example, FIG. 7K indicates angles and dimensions for one variation of an ipsilateral probe, and FIG. 7L indicates angles and dimensions for one variation of a contralateral probe. An ipsilateral probe may be used to treat spinal decompressions using an ipsilateral approach, as described in more detail in co-pending U.S. patent application Ser. No. 12/352,978, titled “MULTIPLE PATHWAYS FOR SPINAL NERVE ROOT DECOMPRESSION FROM A SINGLE ACCESS POINT”, filed on Jan. 13, 2009, Publication No. US-2009-0177241-A1 and fully incorporated by reference herein. Similarly, a contralateral probe may be used to treat spinal decompression using a contralateral approach. The dimensions and measurements provide have been shown to be beneficial. The dimensions (length, width, height, thickness, diameters, etc.) and angles (arc, arc length, curvature, etc.) maybe varied in individual cases +/−1%, +/−2%, or +/−about 5%). The inner cannula may also be sized such that it may be effective in treating spinal stenosis, particularly when operating through a stenotic opening in the spinal nerve channel. In particular, the inner cannula may have a relatively tight radius of curvature such that as it is passed over a nerve root of the spine, it curves above a nerve root rather than through or below the nerve root. Other dimensions have not proven effective in treating spinal stenosis, particularly when operating through a stenotic opening in the spinal nerve channel.

Two of the bends in the ipsilateral probe shown in FIG. 7K are of particular interest: the Sheppard's hook angle (labeled 22°) and the final distal curve of the outer cannula (indicated as 96° off of the long axis of the device). The Sheppard's hook angle allows the probe to move and be positioned while avoiding the spinous process. If this curve is not sufficiently large, the hooked end will be unable to orient the opening in the outer cannula so that the inner cannula can be positioned dorsally. Similarly, the distal curve of the outer cannula should position the outer cannula opening so that the inner cannula exits at approximately 90° (e.g., between about 80° and about 100°) relative to the long axis of the device, as shown in FIG. 7K.

The distal bend in the contralateral probe is typically shallow (having an angle with the long axis of the device of about 128°, as shown. This orientation allows the probe, when inserted contralaterally, to avoid interference by the cauda equina, and other structures, while allowing the probe to be inserted as necessary.

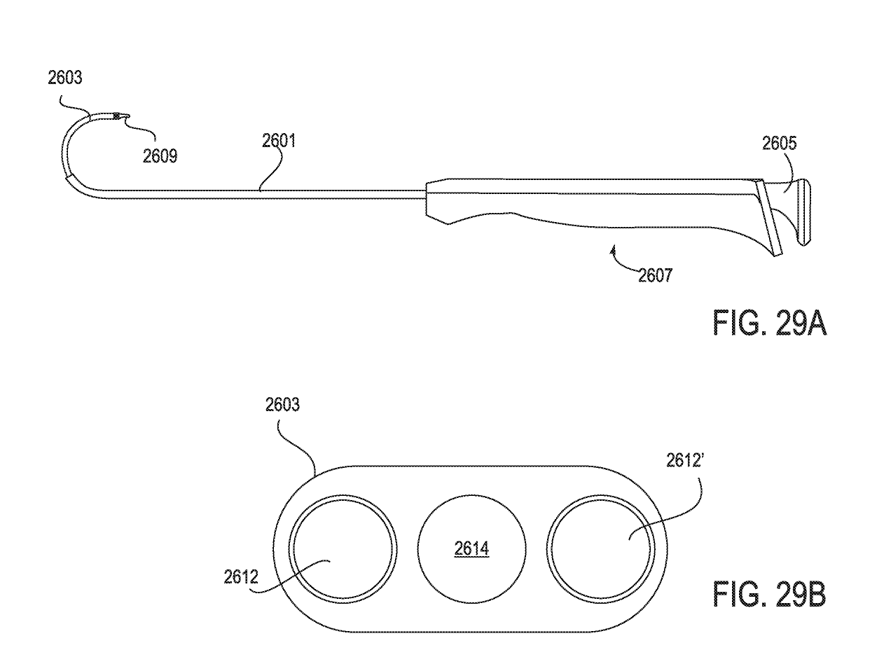

Other probe variations are illustrated and described in greater detail below, for example, in FIGS. 29A-70B. Any of the features described above may be incorporated in these probe configurations, including (but not limited to) the handle features.

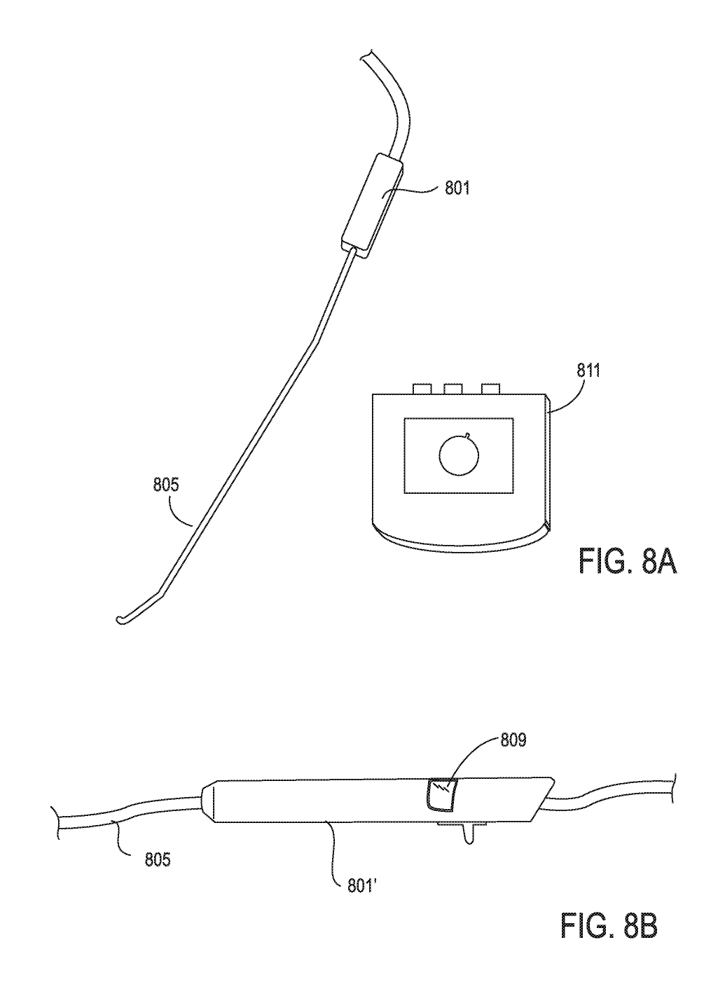

The systems described herein may also include one or more neural localization devices configured to determine the proximity and relative location of a pullwire pathway to a nerve or nerves. For example, FIGS. 8A and 8B illustrate one variation of a neural localization device. In FIG. 8A, the neural localization device includes a proximal handle 801 that is connected to a flexible distal region 805. The device may also include a controller 811 that may be integrated with or separate from the handle 801. The flexible distal end may be configured to couple with a pullwire so that it can be pulled into position. The flexible distal end may include one or more electrodes or arrays of electrodes that may be used to create a limited stimulation field. Stimulation from the distal end of the neural localization device may indicate the presence of a nerve relative to one side or portion of the neural localization device.

In some variations the handle portion 801 of the device may be configured for improved operation, including an indication of what portion (e.g., what side) of the neural localization device is being activated, the orientation of the distal end of the neural localization device, and/or a control for controlling stimulation provided by the neural localization device.

For example, FIG. 8B illustrates one variation of a handle 801′ of a neural localization device. In this example, the handle includes a window 809 on one or more sides of the device that indicates visually where and when stimulation is being applied.

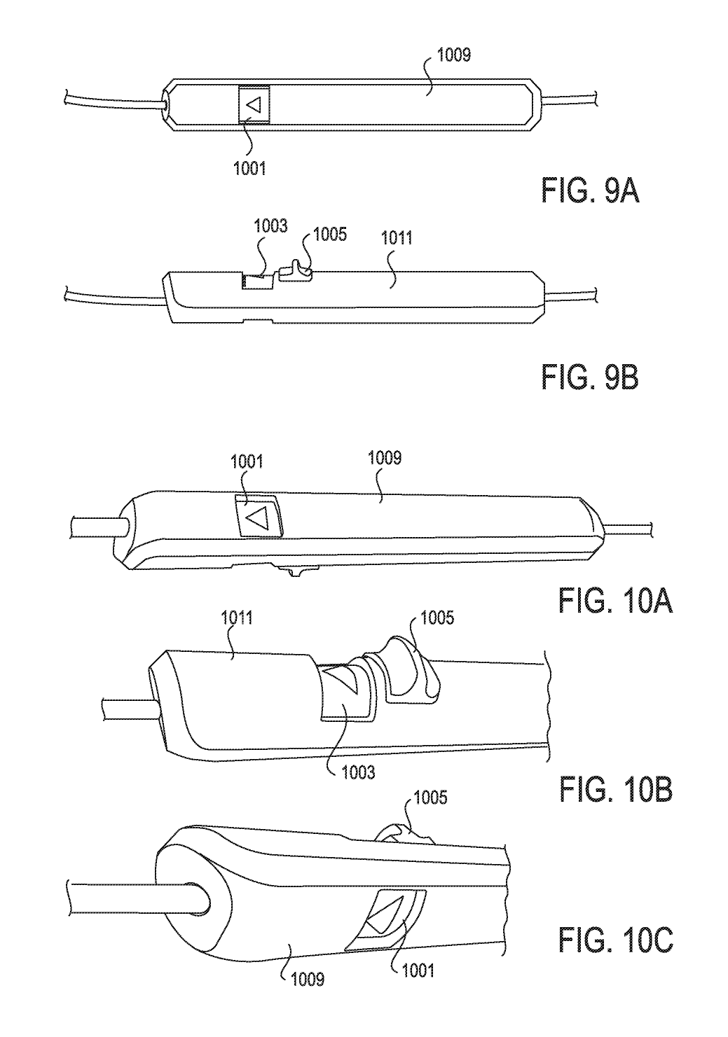

FIGS. 9A and 9B show front and side perspective views of a variation of a neural localization device similar to the device shown in FIG. 8C. For example, the front 1009 of the neural localization device is shown in FIG. 9A. This embodiment includes a window 1001 that indicates when the front or top of the neural localization device is being stimulated. The back 1011 of the handle also includes a window 1003 that indicates when the back or bottom of the neural localization device is being stimulated. The front window 1001 may also indicate when the back of the neural localization device is being stimulated and the back window 1003 may also indicate when the front of the neural localization device is being stimulated. For example, a window may indicate “top”, “bottom”, and/or “off”. The handle also includes a control 1005 (shown here as a slider) for toggling stimulation between the back and front; the control may also be used to turn the stimulation “on” or “off” and in some variations can also be used to determine the level of stimulation.

FIG. 10A shows a side perspective view of the neural localization handle shown in FIGS. 9A and 9B, and FIG. 10B shows an enlarged view of the window on the back side of the handle. FIG. 10C shows an enlarged view of the front window. The window may show an indicator such as a graphic, including a color, an alphanumeric message, a symbol, or the like.

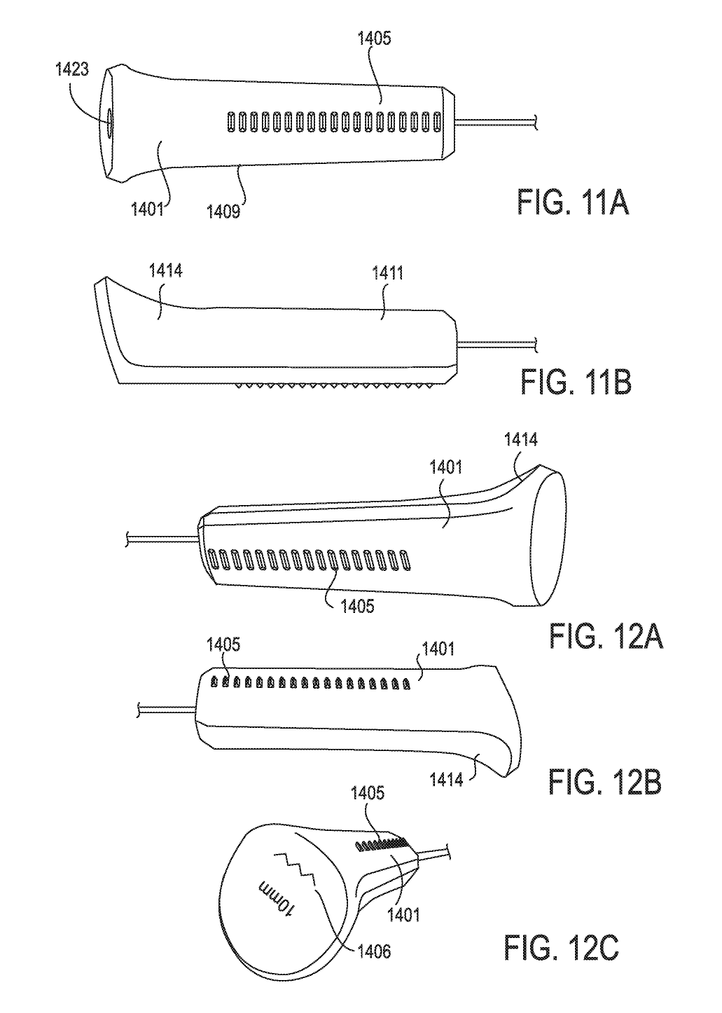

As mentioned above, some variations of the systems described herein also include a tissue modification device such as those mentioned and incorporated by reference above. FIGS. 11A-13D illustrate variations of tissue modification devices, and particularly handles for tissue modification devices. Any of these tissue modification devices may be configured to couple at their distal ends with the proximal end of a pullwire. The handle may include a grip region, and may also include a distinct and flat front face, as mentioned above. The front face may be marked to indicate the orientation of the tissue modification device. Any of the devices described herein, including the tissue modification devices (and neural localization devices, etc.) may be configured so that the orientation of the distal ends of the device are relatively fixed with respect to the handle, and therefore the front face of the handle.

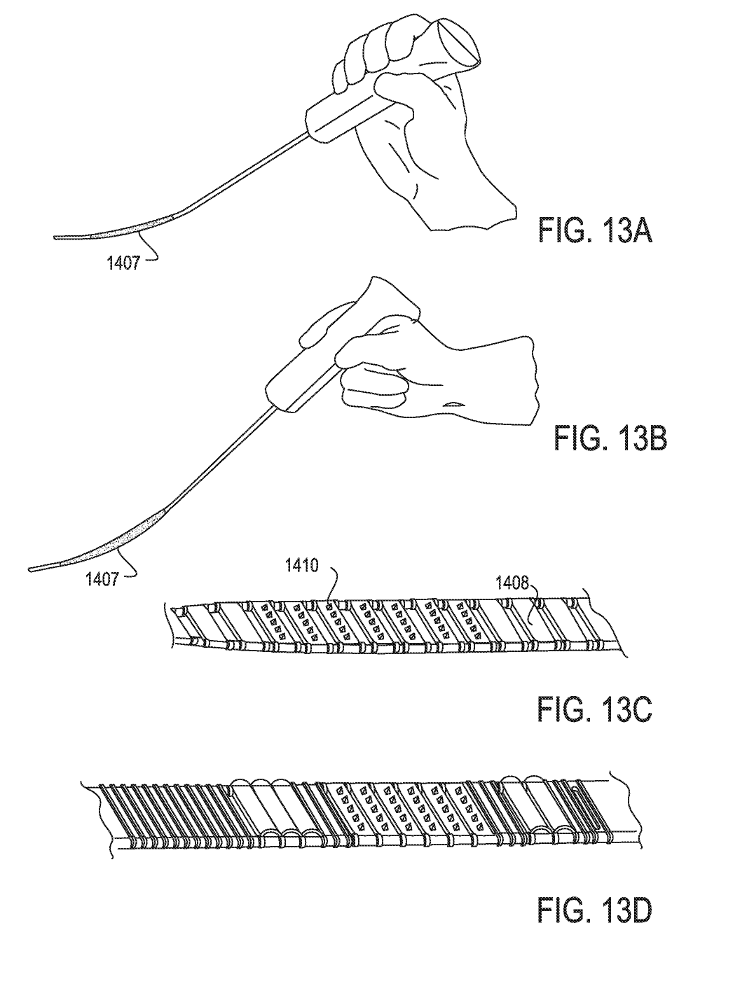

FIG. 11A shows one variation of a handle for a tissue modification device. The handle include the flat front face 1401 that is textured 1405 or otherwise marked to indicate that it corresponds to the front of the device. In this variation, the front of the device predicts the orientation of the distal end of the device including the flexible cutting surface(s), as illustrated in FIGS. 13A-13D. The handle includes a gripping region 1409, and may have smooth or rounded sides 1411. The proximal end of the handle may be shaped to prevent the handle from slipping, including a lip, flange or rim 1414 against which the hand or fingers may rest. The proximal flange 1414 may also be configured so that it does not extend around the front portion of the device, as mentioned above. FIGS. 12A to 12C illustrate alternative views of the handle shown in FIGS. 11A-11B. In some variations the handle may be marked to indicate the size or variation of the tissue modification device, as illustrated in FIG. 12C, in which the proximal end of the handle includes an alphanumeric graphic marking the size (10mm for an example) of the tissue modification device. Furthermore, as shown in FIG. 12C, the handle may be marked to indicate on which side of the device the tissue modification elements are located (e.g. the abrasive or sharp side of the device). As shown, marking 1406 may indicate that the blades or other tissue modification elements are location on the front side 1401 of the device.

FIGS. 13A and 13B illustrate different methods of holding and operating a variation of the handle of the tissue modification device similar to that shown above. For example, in FIG. 13A, the device is configured to be gripped so that the tissue modification device may be driven against the tissue to remove or otherwise modify tissue. In FIG. 13B the device may be held in a penciling configuration. For example, in some variations the handle includes a narrowed or tapered waist configuration. The distal end of the device 1407 is typically flexible, and includes one or more blades or cutters for modifying tissue. For example, FIGS. 13C and 13D illustrate two variations of tissue modification devices that may be used.

FIG. 13C shows a portion of the distal end of a flexible tissue modification device comprising a plurality of flexibly connected rungs 1408 (having a top and a bottom side), that include a plurality of tissue-cutting blades 1410 on some of the rungs. There are gaps between the rungs shown in FIG. 13C, however these gaps are not necessary, as illustrated in the variation shown in FIG. 13D. In FIG. 13D, the device includes a plurality of rungs that are flexible connected without substantial gaps. In this variation some of the rungs include side-cutting blades, configured as side-cutters along the side (widths) of some of the flexibly connected rungs.

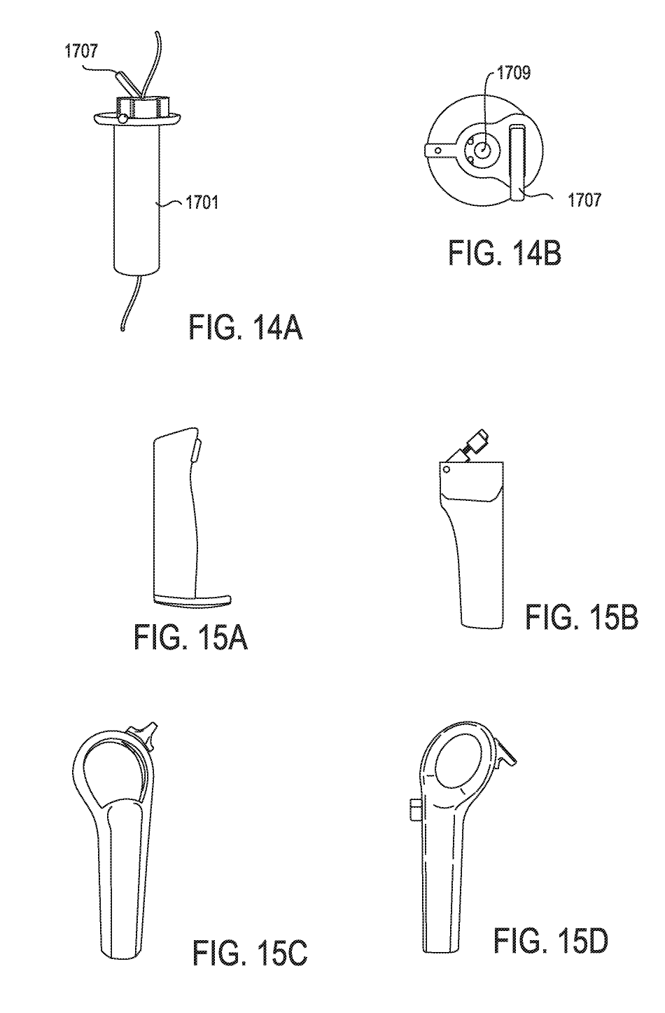

FIGS. 14A to 17C illustrate a distal handle that may be attached to the distal end of a pullwire, and may be included as part of the systems described herein. In general, the handle is configured to removably lock onto a pullwire and allow a substantial amount of pulling (and/or pushing) force to be transmitted from the user through the handle to the pullwire. The handles described herein may be configured so that they may “choke up” on a pullwire, so that the handle can be allowed to freely slide on the pullwire (either forward, moving the handle proximally, or backwards, moving the handle distally). The handles described herein may further be configured so that they may be “choked up” on a pullwire and used to pull a pullwire, all with a single hand. The handle may also control the distal (sharp) end of the pullwire, by storing it within the handle. In some embodiments, as a user chokes up on a pullwire the distal end of the pullwire will be stored within the handle. For example, the further down the handle is moved onto the pullwire, the more of the distal end of the pullwire that is stored within the distal handle. Once the locking mechanism is released, a length of the pullwire can be released from the handle (i.e. “choke-down”).

The devices described herein may be configured to secure/lock the pullwire to the handle so that pulling the handle will result in pulling the pullwire when the pullwire is locked into the handle. The handle may also be configured to prevent kinking of the pullwire within the handle. In general, the pullwire handle (i.e. distal handle) coupled to the pullwire will be used to pull (in a distal direction), not push, the pullwire. Alternatively, a proximal handle (for example, the handle of a tissue modification device or a handle of a neural localization device) may be used to pull the pullwire in the opposite (proximal) direction.

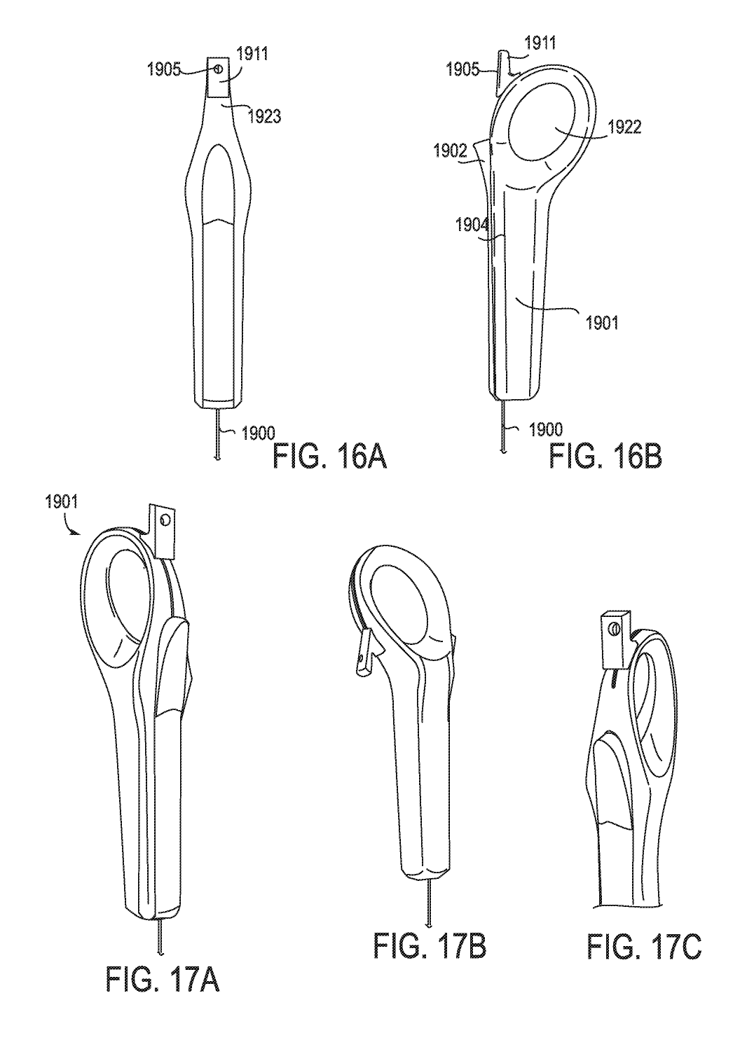

For example, FIG. 14A shows one variation of a distal handle 1701, including a central passageway 1709 through which the pullwire may be passed, and including a lock 1707 for locking the pullwire within the passageway. The lock 1707 is shown in FIG. 14B as well. FIG. 14B is a top view of the handle shown in FIG. 14A. The handle may also include a pullwire capture chamber 1922, as shown in FIG. 16B, for securing the (typically sharp) distal end of the pullwire which may otherwise pose a danger to the user. In this variation the pullwire handle may slide over the distal end of the pullwire, and then the pullwire may be looped through the pullwire capture chamber and be locked in position so that the handle may then be used to apply force to pull the pullwire distally (and thereby manipulate any of the devices described above, that may be coupled distally to the proximal end of the pullwire.

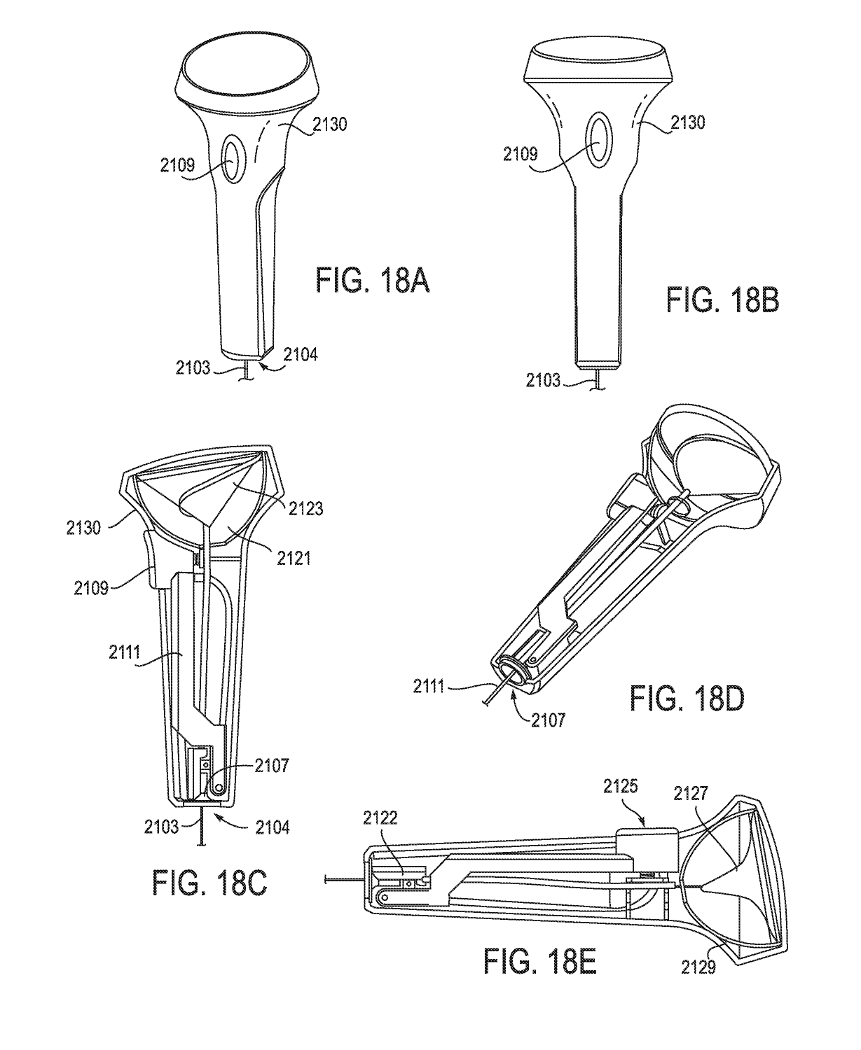

FIG. 15D illustrates another variation of a pullwire handle. In this variation, the pullwire handle includes a central lumen into which the distal end of the pullwire may be fed. The end of the handle may be open, and may be tapered, convex, concave, or otherwise funnel-shaped (as shown in FIG. 18D) so that the pullwire can readily be captured. In this variation, the handle including a tip capturing element that allows the distal end of the handle to be held in the handle while it is advanced (e.g. proximally). The handle may also include a latch or lock for securing the pullwire within the handle at a certain position. In some variations the handle (as shown in FIG. 15D) includes a pathway that loops the pullwire around the handle, and out of the way automatically. In some variations the handle includes a spool or path into which the pullwire may be threaded to keep it from interfering with the operation of the handle and/or pullwire.

FIGS. 15A-15D illustrate alternative variations of the pullwire handle described, and FIG. 15D shows the same variation described in FIG. 15D.

FIGS. 16A and 16B show font and side views, respectively, of the pullwire handle just described with reference to FIG. 15D. In this variation, the pullwire 1900 is first fed into the distal end of the pullwire handle which may include a funnel-shape (not shown) that communicates with an opening or internal track through the handle. This variation of a pullwire handle also includes one or more buttons or controls 1902. For example, the handle device 1901 may include one or more controls 1902 that lock or release a pullwire held within the handle, preventing or allowing the handle to slide on the pullwire. Thus, a handle may be inserted onto the distal end of a pullwire and chocked up on the pullwire. In some variations, the device includes an indicator (e.g. a visual indicator or window 1905) showing when the distal end of the pullwire is loaded within the handle. The pullwire handle may also include a loader or feeding control 1911 for advancing or loading the pullwire into the handle. In FIGS. 16A and 16B, the loader control 1911 is configured to hold the distal end of the pullwire (visible in the window 1905) and be pushed or moved along the rounded perimeter of the handle 1922 and draw the pullwire further into and around the handle, as mentioned. This curved or rounded distal end includes a channel or opening (e.g., a split) 1923 from which a portion (e.g., a portion proximal to the captured distal end) may exit the handle as a loop as the handle is choked up on the pullwire. The pullwire handle may also include a flat or marked front face 1904, as mentioned above.

FIG. 17A shows a side perspective view of the pullwire handle 1901, and FIGS. 17B and 17C show side and enlarged front (distal) views as of the same variation.

Another variation of a pullwire handle is shown in FIGS. 18A-18D. In this variation, the pullwire handle includes a proximal end 2104 into which a pullwire 2103 is fed. As mentioned above, this proximal end may be conical, funnel-shaped, or otherwise shaped to easily capture and guide the distal end of the pullwire into the handle. In some variations the proximal end is flared (e.g., trumpet shaped) or includes a flange or rim (not shown in FIGS. 18A-18D), which may help steer the pullwire into the handle and avoid injury from the often sharp end of the pullwire.

This variation also includes a control 2109 (shown as a button 2109 in FIGS. 18A and 18B) that may control the locking/unlocking of the pullwire in the handle. For example, the button may be pushed to unlock the pullwire, allowing it to be advanced into the handle, or withdrawn from the handle. In some variations, the control may be pressed or activated continuously to unlock (e.g., maintaining the hold on the button), while in other variations the control may be engaged to remain either locked or unlocked.

The handle may store the pullwire as it is inserted into the device. For example, the device may include a storage region for holding the pullwire, such as a spool around which the pullwire may be wound. This is illustrated in FIGS. 18C and 18D.

FIG. 18D shows a side view of the device of FIGS. 18A and 18B, made partially transparent to reveal the inner structures. In this variation, the pullwire 2103 may be fed from the proximal end 2104 (having a funnel-shaped feed region 2107). Pressing the button 2109 releases a lock mechanism that includes lever arm 2111 that otherwise restricts the movement of the pullwire out of the handle. In some variations, the handle may allow advancement of the pullwire into the handle even when the device is ‘locked,’ and only prevents withdrawal of the pullwire from the handle when the device is locked. The distal end of the handle includes a storage region 2121 having a spool 2123 that winds the pullwire around within the storage region. The spool may be fixed or rotating. In some variations the handle may include a control for winding the pullwire within the handle. For example, the handle may include a crank, lever, button, or the like, for rotating the spool and drawing the pullwire into the handle (e.g., into the storage region 2121). In one variation the distal end of the handle (e.g., the flared or bell-shaped distal end 2130) is rotatable.

Another view of this variation of a removable pullwire handle is shown in FIG. 18E. As described above, a pullwire may enter the handle from the distal end (which has a concave or funnel-shaped opening), and is fed through the handle in a channel that leads to the storage area that is cup-shaped 2129. This cup region 2129 may enclose a surface that directs the pullwire around the chamber so that it can be wound up and stored therein. In FIG. 18E the surface is an inverted cone 2127 that deflects the pullwire so that as the pullwire is advanced, it is forced against the cup wall. Once the pullwire hits the cup wall, the pullwire buckles and wraps around the smooth interior of the cut. This variation also illustrates a locking clamp 2122 that is positioned at the proximal end and may lock or hold the pullwire within the handle housing. A control (e.g., push button) 2125 on the handle activates/deactivates the clamp or lock. For example, in FIGS. 18A-18E, the control is a push button 2125 that articulates a lever arm inward, which releases the clamp 2122 at the bottom of the handle. This opens the clamp, and releases tension on the pullwire. When the bottom is released, a spring inside the button (“thumb button”) forces the lever arm back into position, thereby closing the clamp and increasing the tension on the pullwire.

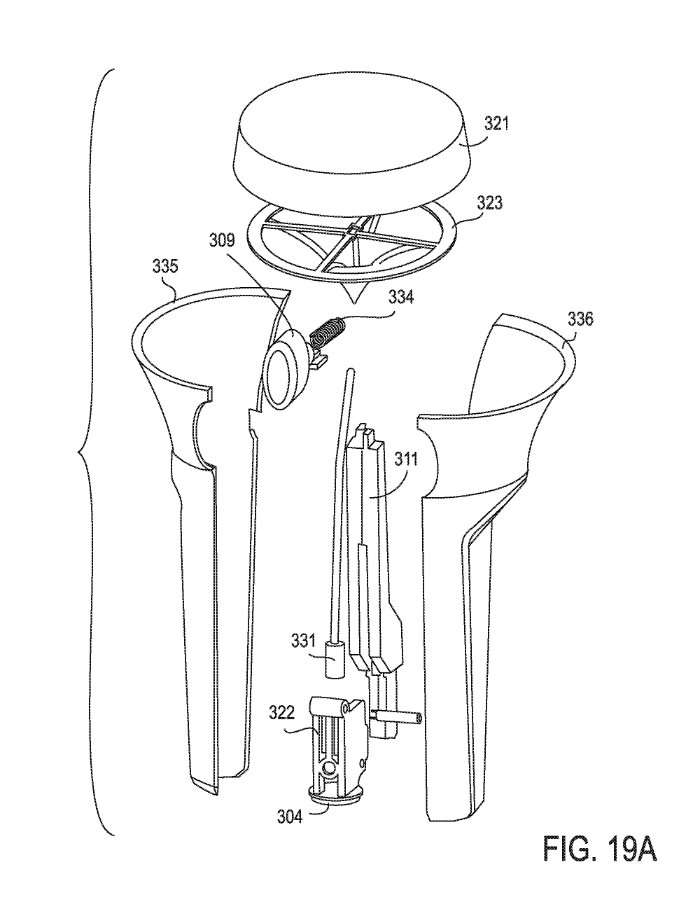

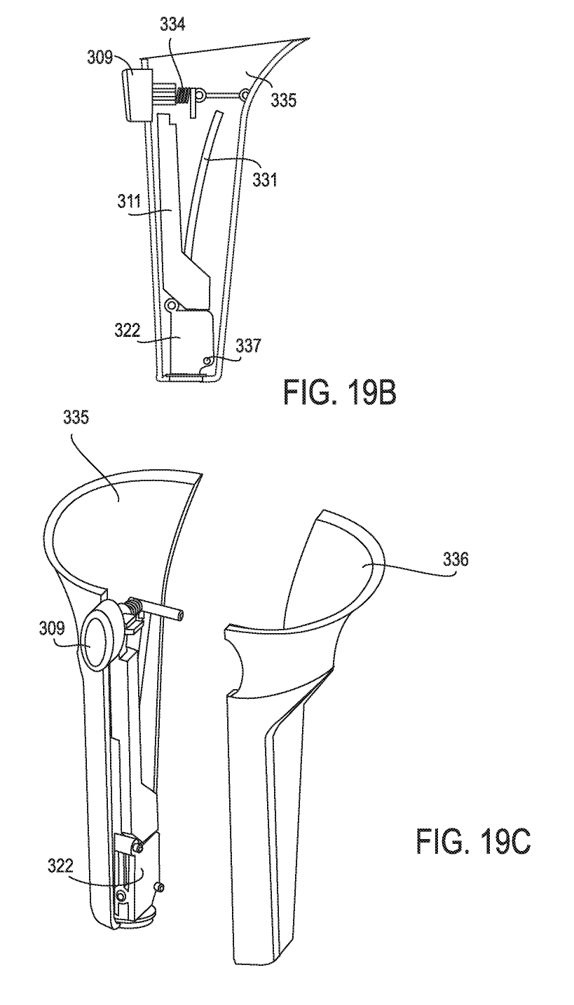

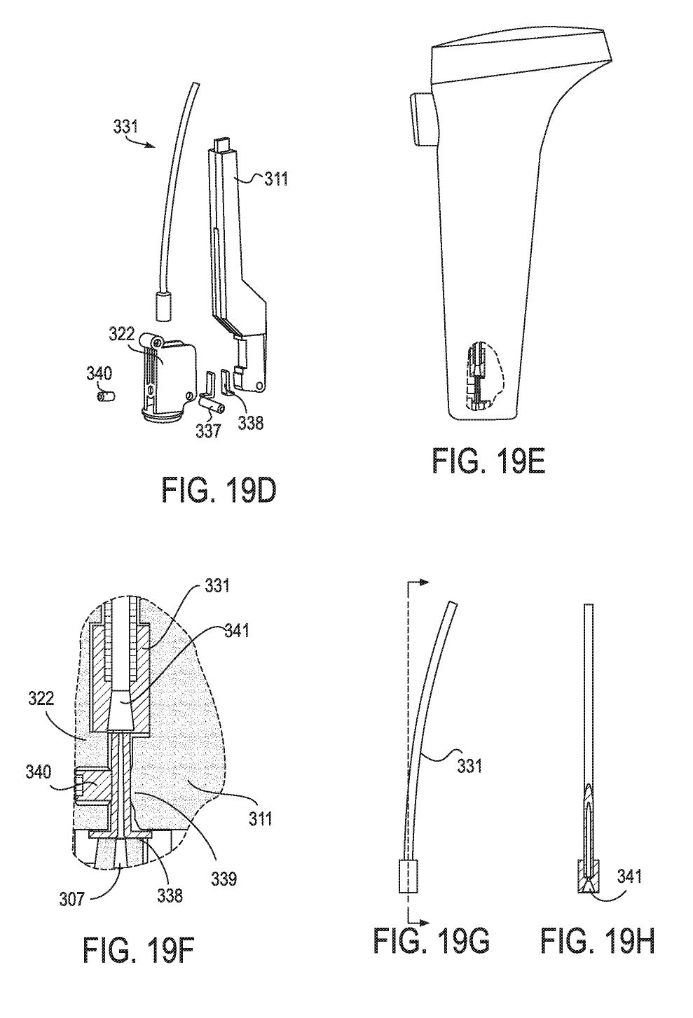

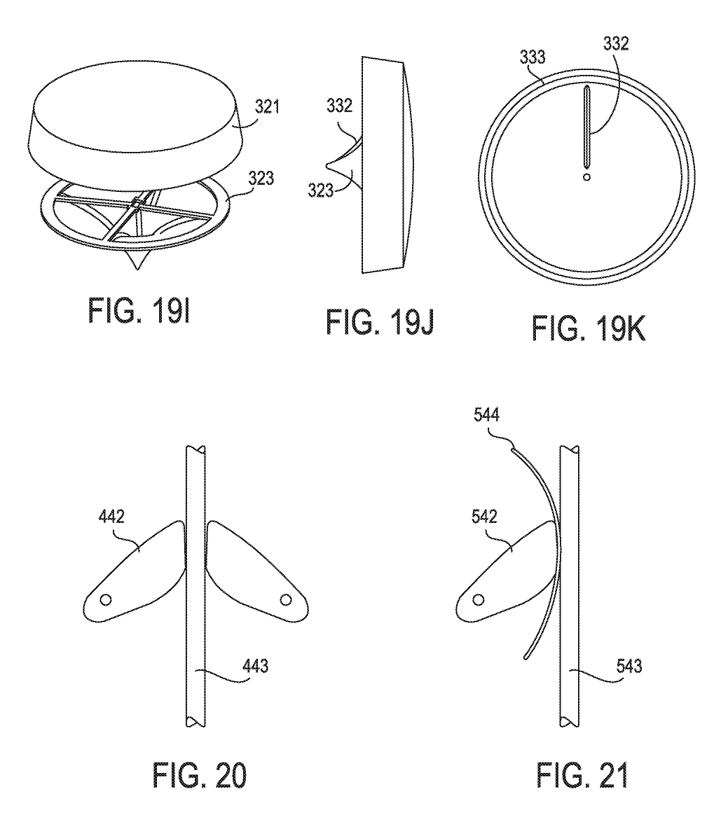

FIGS. 18A to 19K illustrate a distal handle that may be attached to the distal end of a pullwire, and may be included as part of the systems described herein. The distal handle is configured to hold the distal end pullwire such that a user may pull on the pullwire, and the device that it is attached to, with a large amount of force (e.g., enough force to modify tissue such as bone and cartilage). The distal handle may hold the pullwire with a lock mechanism that is configured to withstand forces within the range of 10 lbs to 60 lbs. For example, the handle may be configured to lock and secure the handle so that it can transmit up to 40 b of force, up to 50 lb of force, up to 60 lb of force, up to 100 lb of force, etc. without “slipping.” Slipping may refer to the release of the lock so that the handle moves relative to the pullwire. Furthermore, the handle may be configured to lock and secure the handle so that it can transmit large forces without kinking or bending the wire. For example, as described in detail below, the locking mechanism in some embodiments may not damage, modify, or non-elastically bend the pullwire while still locking onto the pullwire with a great enough force such that the pullwire/distal handle can transmit a large amount of force to the proximal end of a pullwire and/or device coupled to the pullwire. In general, it may be important to transmit a large amount of force to the pullwire via the pullwire handle in order to pull a proximal end of the pullwire (and/or a device coupled to the proximal end of the pullwire) through a tight or confined space. For example, the pullwire and pullwire handle may be used to pull a tissue modification device coupled to the pullwire through a stenotic spine of a patient.

In manufacturing of the described pullwire handle, the pullwire handle may be designed for a maximum holding strength. Adaptations configured to assist with the locking strength of the handle include the materials chosen, surface finishes of the materials chosen, the shape and dimensions of the interface between the handle and the pullwire, the number of locking mechanisms (e.g., cams, etc.), the surface properties of the locking interface, mechanisms such as springs coupled to the locking mechanisms or gripping surfaces that increase clamping force, and the like. Examples of such features are described in greater detail below. For example, FIGS. 18A and 18D show one variation of a distal handle. In this variation, the pullwire handle includes a proximal end 2104 into which a pullwire 2103 is fed. As shown in FIG. 18D, this proximal end may be conical, funnel-shaped, or otherwise shaped to easily capture and guide the distal end of the pullwire into the handle. FIG. 18D shows a perspective view of the device of FIG. 18A, made partially transparent to reveal the inner structures. In this variation, the pullwire 2103 may be fed from the proximal end 2104 (having a funnel-shaped feed region 2107).

This variation also includes a control 2109 (shown as a button 2109 in FIG. 18A) that may control the locking/unlocking of the pullwire in the handle. For example, the button may be pushed to unlock the pullwire, allowing it to be advanced into the handle, or withdrawn from the handle. In some variations, the control may be pressed or activated continuously to unlock (e.g., maintaining the hold on the button), while in other variations the control may be engaged to remain either locked or unlocked. The handle may store the pullwire as it is inserted into the device. For example, the device may include a storage region 2130 for holding the pullwire, such as a spool around which the pullwire may be wound. In some embodiments, the methods of using the handle described above may include the steps of depressing the button to release the locking mechanism, inserting the distal end of a pullwire into the pullwire handle device, storing a portion of the pullwire within the pullwire handle device, and releasing the button to activate the pullwire lock to lock the pullwire within the handle.

Another variation of a pullwire handle is shown in FIGS. 19A-19E. This variation of the distal pullwire handle is similar to the variation of FIGS. 18A and 18D. FIG. 19A shows an exploded view of this variation. As shown in FIG. 19A, the pullwire handle of this variation includes a first housing portion 335 and second housing portion 336. The housing portions may be coupled together in a clam shell configuration, or may be coupled together in any suitable configuration to form the housing of the handle and enclose the various components of the handle. In some embodiments, the housing portions may make up the handle body of the pullwire handle device. The pullwire handle (and/or handle body) also includes proximal end 304 into which a pullwire is fed. As mentioned above, this proximal end may be conical, funnel-shaped, or otherwise shaped to easily capture and guide the distal end of the pullwire into the handle. This variation also includes a control 309 (shown as a button 309 and spring 334 in FIGS. 19A-19C and 19E) that may control the locking/unlocking of the pullwire in the handle. For example, a lock mechanism of the pullwire handle may be nominally locked (i.e. always locked until unlocked by the control) and the button may be pushed to unlock the pullwire, allowing it to be advanced into the handle, or withdrawn from the handle. In some variations, the control may be pressed or activated continuously to unlock (e.g., maintaining the hold on the button), while in other variations the control may be engaged to remain either locked or unlocked.

In this variation, the handle stores the pullwire as it is inserted into the handle. As shown in FIG. 19A, the device (and/or handle body) includes a storage region 321 for holding the pullwire and a cone 323 that guides the pullwire against the inner diameter of the storage region 321. In this variation, the pullwire may be fed from the proximal end 304 (having a funnel-shaped feed region 307, as shown in FIG. 19F). Pressing the button 309 releases a lock mechanism that includes lever arm 311 that otherwise restricts the movement of the pullwire out of the handle. As the pullwire is fed from the proximal end 304, it runs through the pullwire guide tube (or internal track) 331. The pullwire exits the distal end of the pullwire guide tube such that it contacts the cone 323. As the pullwire exits the tube and contacts the cone, the geometry of the cone guides the tip of the pullwire toward the inner diameter of the storage region 321.

As shown in FIGS. 19I-19K, the cone 323 includes a surface that directs the pullwire around the chamber of the storage region 321 so that it can be wound up and stored therein. The surface deflects the pullwire so that, as the pullwire is advanced, it is forced against the inner wall of the chamber. Once the pullwire tip hits the chamber wall, the pullwire buckles and wraps around the inner diameter of the storage region chamber. In some variations, the surface deflects the pullwire so that as the pullwire is advanced, it is forced against a ridge 332 on the cone. Once the end of the pullwire hits the ridge 332, the pullwire buckles and wraps around the inner diameter of the storage region chamber. Alternatively (or additionally), the ridge 332 may function to guide the pullwire, particularly after it has begun to buckle, away from the cone 323 and to continue to wrap within the chamber 321. The ridge 332 may therefore function to prevent the pullwire from hitting the chamber wall and buckling back onto cone 323 and jamming against the cone. Alternatively, the surface deflects the pullwire so that as the pullwire is advanced, it is forced against a ridge or pin 333 on the inner diameter of the chamber of the storage region. Once the end of the pullwire hits the ridge or pin 333, the pullwire buckles and wraps around the inner diameter of the storage region chamber.

FIG. 19B shows a partial cross sectional view and FIG. 19C shows a partially exploded view of this variation. This variation also illustrates a clamp housing 322 that is positioned at the proximal end and encloses the clamp 338 (as shown in FIGS. 19D-19F) that may lock or hold the pullwire within the handle housing. A control (e.g., push button 309 coupled to spring 334) on the handle activates/deactivates the clamp or lock. The push button 309 articulates a lever arm 311 outward and/or clockwise about a pin 337, which releases the clamp at the bottom of the handle. This opens the clamp, and releases tension on the pullwire. When the button is released, the spring 334 forces the lever arm 311 back into position, thereby closing the clamp and increasing the tension on the pullwire.

FIG. 19D shows an exploded view of the lock mechanism of the pullwire handle. FIG. 19E shows a partial cross-section through the handle and FIG. 19F shows a detail view of the section of FIG. 19E. As shown, this variation of the lock mechanism includes a clamp housing 322. The clamp housing, as shown in FIGS. 19D-19F includes a funnel-shaped feed region 307 at the proximal end of the handle into which the pullwire is fed. The clamp housing 322 also houses the clamp 338 and clamp adjustment set screw 340. The clamp functions to provide an increased surface area with which to grip and lock the pullwire into place. The increased surface area may also help to prevent damage, kinking, or bending of the pullwire as described above. In some embodiments, the clamp is designed to “float” within the clamp housing. As the tension of the pullwire increases (i.e. a user is pulling on the pullwire handle with increased force applied to the pullwire or the user simply pulls the pullwire handle in a distal direction to place the pullwire in tension), the clamp slides towards the proximal end of the handle thereby, allowing the cam to further rotate towards the pullwire; thereby, increasing the holding and locking strength on the pullwire.

Alternatively, rather than including a clamp, the cam surface 339 (as described below) could directly contact the pullwire, or a shim or other suitable strip of material may be used to increase the surface area upon which the cam surface may apply a force to the pullwire to hold the pullwire in place. The clamp adjustment set screw 340 may be threaded (or otherwise coupled to) the clamp housing 322 and may function to tighten or loosen the arms of the clamp 338. The clamp housing is coupled to the lever arm 311 with pin 337 about which the lever arm rotates.

As shown, this variation of the lock mechanism also includes a lever arm 311. At the distal end of the lever arm, the lever arm is coupled to button 309. At the proximal end of the lever arm, the lever arm includes cam surface 339 and, as mentioned above, is coupled to the clamp housing 322 via pin 337. As mentioned above, the push button 309 articulates the lever arm 311 outward and/or clockwise about a pin 337. This rotates the cam surface 339 away from the clamp 338 which releases the clamp and allows the clamp to open up and release tension on the pullwire. When the bottom is released, the spring 334 forces the lever arm 311 back into position, thereby rotating the cam surface 339 against the clamp, effectively closing the clamp and increasing the tension on the pullwire.

In any of the variations described herein, the locking mechanism in the handle that holds the pullwire may be a clamping mechanism that includes a gripping surface. This gripping surface may be sized so that it provides significant surface contact with the pullwire from two or more sides. For example, the gripping surface may include a shim surface that is substantially parallel to another (e.g., a fixed or a similar clamp) surface, between which the pullwire passes and is gripped when the lock/clamp is activated. This surface may be a separate element (e.g., a shim element) or it may be a portion of the locking arm or cam, as described and exemplified below. In some variations the clamp/lock mechanism includes a tubular surface or sleeve into which the pullwire passes and is gripped, for example, by compressing or inflating the sleeve/surface. The surface(s) that are locked against the pullwire may be textured to aid in gripping the pullwire. For example, the surface may be rough or may include channels (e.g., transverse channels). The surface(s) may be treated, coated, or formed of a grip-enhancing material having a relatively higher surface friction when engaging the pullwire. For example, the surface may be formed of a tacky or sticky material. As will be described below, a gripping/clamping surface may also be configured to prevent “kinking” of the pullwire in the lock.

The gripping/clamping surface may be any appropriate length. The length may depend upon the locking mechanism. For example, when multiple locking mechanisms are used, the surface may be longer. In some variations the surface(s) is between about 1 mm (or 2 mm, 3 mm, 5 mm, 10 mm, 15 mm, 20 mm, 50 mm, etc.) and about 2 mm (or about 3 mm, 5 mm, 10 mm, 15 mm, 20 mm, 50 mm and 100 mm).

FIGS. 19G and 19H show a side and cross sectional view respectively, of a pullwire guide tube (or internal track) 331. As shown in cross section in FIG. 19H, the pullwire guide tube of this variation includes a proximal end 341 into which a pullwire is fed as it exits the clamp and clamp housing as described above. Proximal end 341 is configured to capture and align the tip of the pullwire as the pullwire passes over the clamp blocks 338. As shown, this proximal end 341 may be conical, funnel-shaped, or otherwise shaped to easily capture and guide the distal end of the pullwire into the pullwire guide tube 331 and up to the storage region. FIG. 20 shows an alternative embodiment of a lock mechanism including more than a single cam surface. This embodiment includes two opposing cams 342 that rotate toward one another (curved arrows) to grip and hold a pullwire 343. In this embodiment, the lock mechanism may not include a lever arm and/or control button. Alternatively, the lock mechanism may be a passive lock mechanism such that as the pullwire moves in the proximal direction (i.e. out of the handle) the pullwire pulls and rotates the opposing cams distally, or downward (as shown by the down arrow in FIG. 20). The cams in this embodiment are configured such that as they are rotated downward, they apply more and more force onto the pullwire 343, thereby locking the pullwire into place. If the pullwire is pushed up (or distally) however, the cams will rotate in the opposite direction, and will allow the pullwire to be moved in that direction (i.e. into the handle). In some embodiments, the mechanism may include a plurality of opposing cam pairs.

FIG. 21 shows an alternative embodiment of a lock mechanism including a shim element 544. As mentioned above, the shim element 544 functions to distribute the force from the cam 542 along a larger surface area of the pullwire 543. A shim element 544 (or other elongate locking/contacting surface) may also prevent the pullwire 543 from kinking under the applied within the lock mechanism. In some variations the length (along the long axis that the pullwire extends) and the width (perpendicular the long axis) may be selected to prevent kinking. In variations in which a separate shim element (e.g., clamping surface/element) is not used, the cam or locking mechanism may be configured to contact the pullwire directly and may be configured to have an integral pullwire contacting surface, as described above. In such variations the increasing the diameter of the contacting surface of the cam mechanism may also function to prevent the pullwire 543 from kinking under the applied force within the lock mechanism. In some embodiments, the shim element 544 may be made from a stainless steel or other suitable metal material, while the cam 542 may be made from a plastic or other suitable molded material. This may reduce cost and/or increase reliability of the lock mechanism.

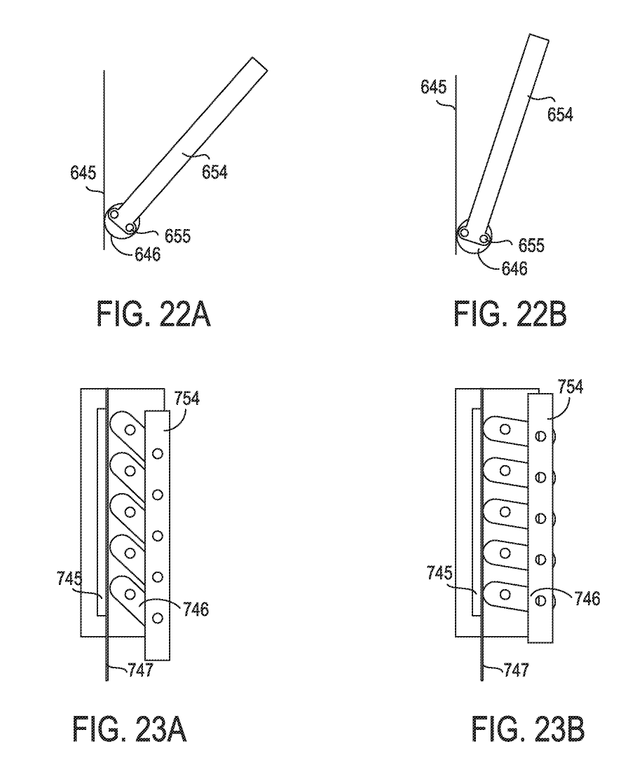

FIGS. 22A and 22B show an alternative embodiment of a lock mechanism including a cam mechanism. As shown in FIG. 22A, the cam mechanism includes a fixed surface 645 and a cam surface 646. In FIG. 22A, the cam mechanism is in an open position, wherein the pullwire may pass through the mechanism between the fixed surface 645 and a cam surface 646 up into the pullwire handle. A lever arm 654 is coupled to the cam surface 646 such that, as shown in FIG. 22B, as the lever arm rotates inward and/or counter-clockwise, this rotates the cam surface 646, about pin 655, toward the fixed surface 645 and applies a force on the pullwire, for example, locking it in place. Additionally, in this configuration, as the wire is pulled out of the distal handle and/or the distal handle is pulled off of the pullwire the cam mechanism is configured to tighten on the pullwire as it is pulled down and/or out of the distal handle. As mentioned above, any of these variations may also include a shim or other contact/gripping element and/or surface.

In alternative variations, the locking mechanism may include multiple cam surfaces and/or multiple lever arms to increase the locking force applied to the pullwire. The pullwire in this variation is locked by series of cam surfaces which provide redundancy in holding wire. An initial clamp force may be provided by a spring or button (as described above) coupled to a lever arm for example, forcing the cams against the wire. A secondary clamping force may be provided as the wire is pulled out of the distal handle and/or the distal handle is pulled off of the pullwire, wherein the wire is effectively pulling down on clamp mechanism. The harder the wire is pulled the more force the clamp applies. As shown in FIG. 23A, the lock mechanism includes multiple cams 746 coupled to a single arm 754. The cams may function to grip a pullwire 747 against surface 746. In FIG. 23A, the cam mechanism is in an open position, wherein the pullwire 747 may pass through the mechanism between the fixed surface 745 and a cam surface 746 up into the pullwire handle. Arm 754 is coupled to the cam surfaces 746 such that, as shown in FIG. 23A, as the arm moves upward, the cam surfaces rotate counter-clockwise and/or toward the fixed surface 745 locking pullwire 747 into place. Once locked into place, as shown in FIG. 23B, the arm 754 may be moved down such that the cam surfaces rotate clockwise and/or away from the fixed surface 745 releasing the pullwire 747. In some embodiments, the cams 746 may all be directly coupled to arm 754 such that the cams move in unison. Alternatively, the cams coupled to arm 754 such that they have the ability to “float” as described above. In other words, as the tension of the pullwire increases (i.e. a user is pulling on the pullwire handle with increased force applied to the pullwire), the “floating” cams further rotate; thereby, increasing the holding and locking strength on the pullwire. The “floating” cams may be coupled to arm 754 such that when the cams are moved to the open, or disengaged position, they all open in unison or at about the same time.

Any of the handle variations described herein, including that shown in FIGS. 21-23B, may include a disengaging/engaging control configured to release the lock. For example, in FIG. 23B, a disengaging/engaging control may be configured to move the locks (e.g., cams) towards or away from the fixed surface 745.

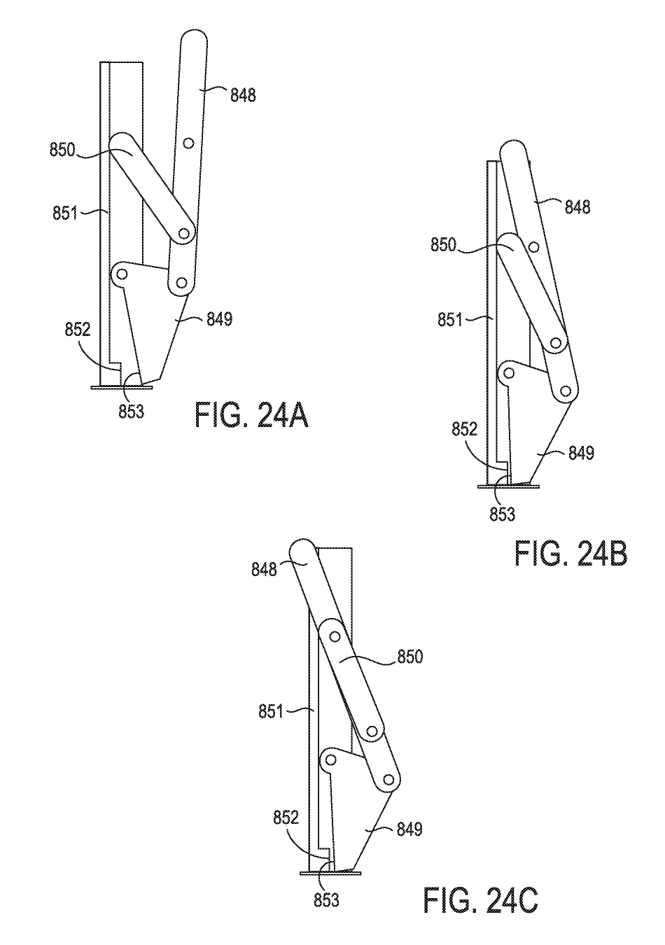

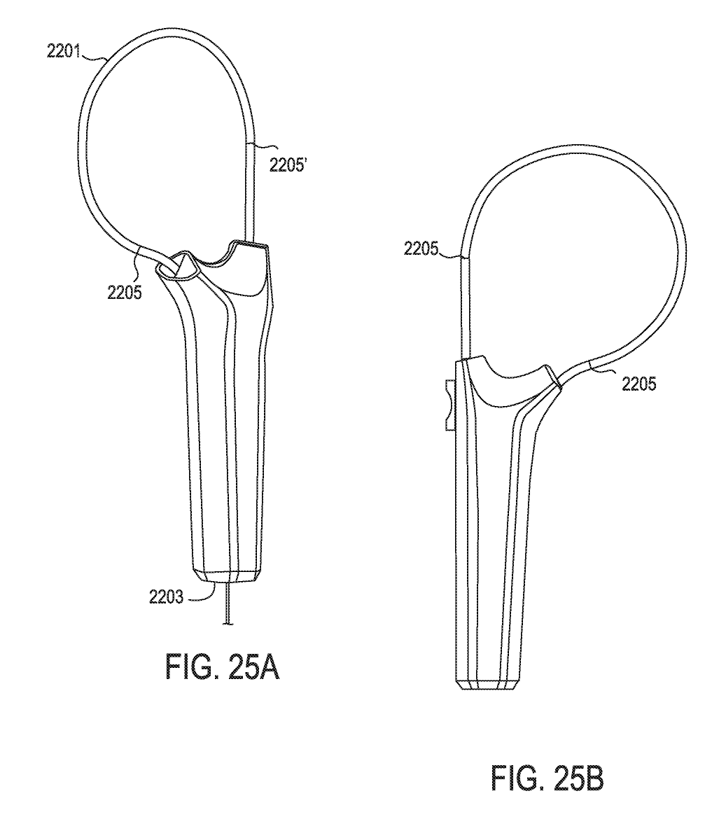

In alternative variations, as shown in FIGS. 24A-24C, the locking mechanism comprises a four bar linkage, such as an over-center four bar linkage. In this variation, the four bar linkage mimics a Vise-grips or locking pliers. The four bar linkage includes bars 848, 850, 849, and 851. Bars 849 and 851 include gripping surfaces 853 and 852, respectively. Similar to Vise-Grips, the lock mechanism can lock the pullwire into position using an overcenter action. Bar 848 functions as a lever. As shown in FIG. 24A, bar 848 is in an open position. As bar 848 is pushed toward bar 851, as shown in FIGS. 24B and 24C, the linkage forces the gripping surfaces toward one another with a large amount of force. As shown in FIG. 24B, bar 848 is moved such that it is moved over its center point (i.e. overcenter) and the linkage locks the jaw (bars 849 and 851) of the pliers onto the gripped object. The bars may alternatively be arranged in any other suitable configuration to provide a large gripping or locking force between at least one gripping surface and the pullwire.

FIGS. 25A and 25B illustrate another variation of a removable/adjustable handle for the distal end of a pullwire. In this variation the distal end includes a guide channel 2201 through which the pullwire may be fed. Once the pullwire is passed from the funnel-like opening in the proximal end 2203 of the handle (not visible), it may pass through the body of the handle, and into and around this loop region. The user is protected from the sharp distal tip, because it is within the body of the handle (and this distal loop). As the distal end of the pullwire passes out of the loop and back into the solid region of the handle, it may be held (releasably) in the handle. However, if the handle is further “choked-up” on the pullwire such that more pullwire is fed into the handle, the distal tip of the pullwire will remain within the handle, however the loop 2201 may ‘break away’ from the body of the handle, as shown in FIGS. 23C-23E, allowing more and more pullwire 2310 to be threaded distally into the handle. This progression can be seen in FIGS. 23B, 23C, 23D, and then 23E. The break-away lock may therefore allow the loop of pullwire to continue to be taken up, while controlling the sharp tip. Thus, in some variations, this loop may be frangibly connected to the body of the device, or it may include a breakable region 2205, 2205′, so that pressure from the pullwire within may allow it to separate after guiding the tip of the wire into the handle and forming a loop that can then expand further.

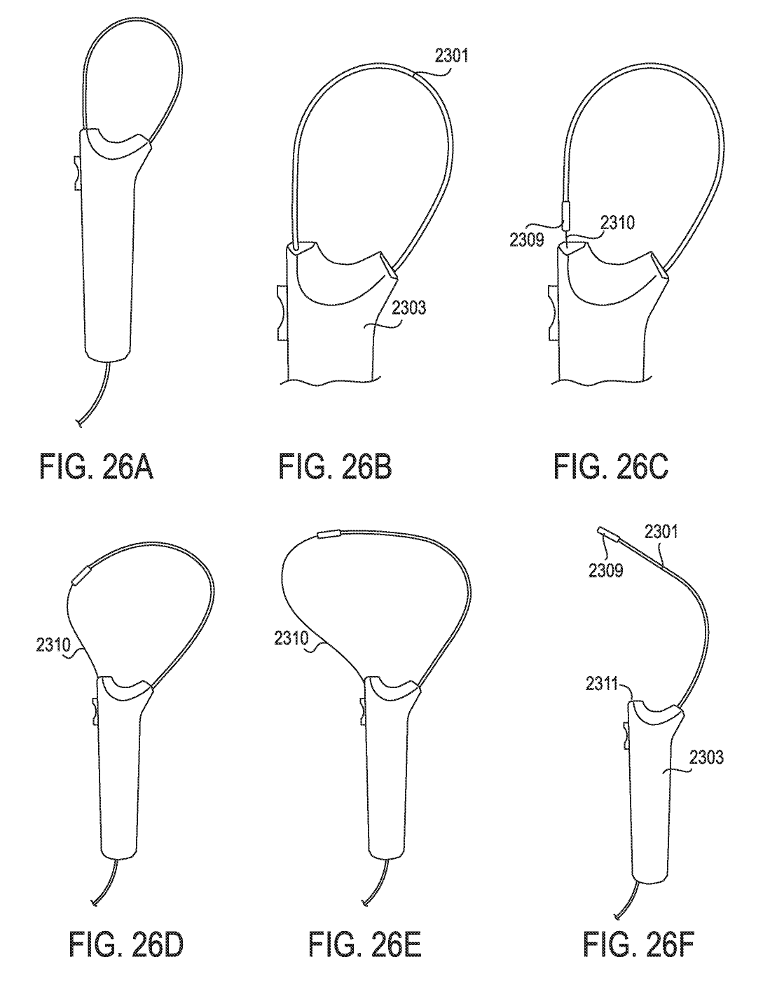

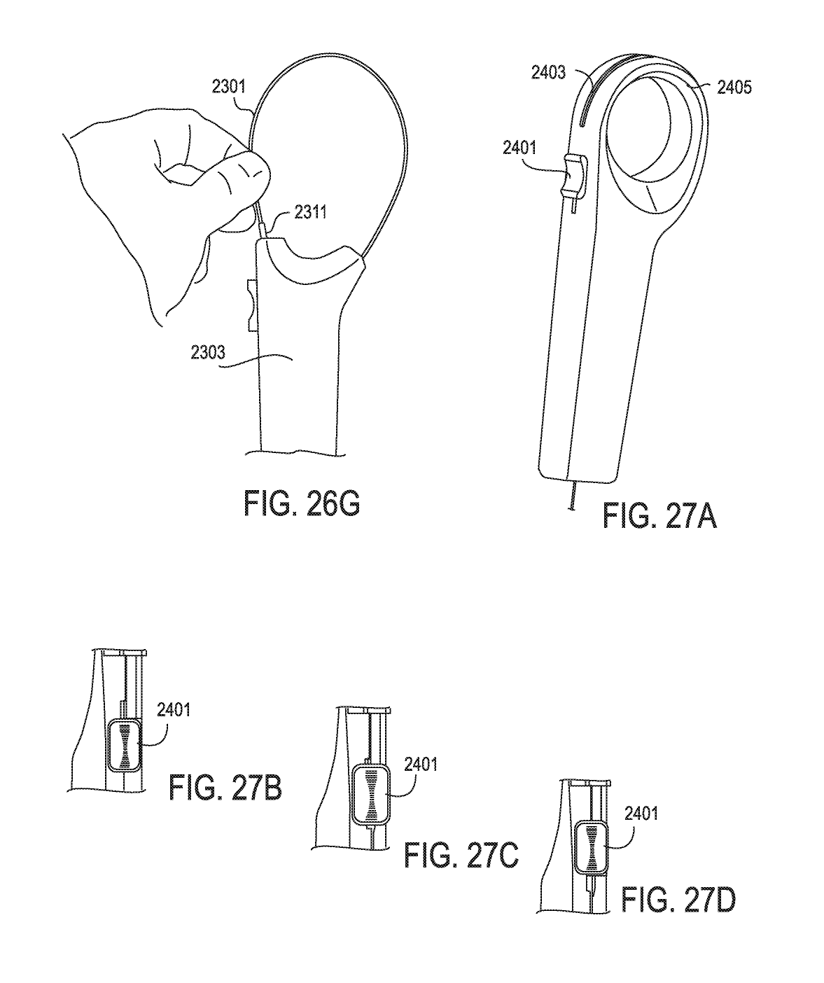

Another variation of a pullwire handle having a distal pullwire handle that is removable and reusable (similar to the one described above in FIGS. 25A and 25B) is shown in FIGS. 26A-26G. For example, FIG. 26A shows a pullwire handle having a separable guide region 2301 for a pullwire. FIGS. 26B-26G illustrate operation of this variation. For example, in FIG. 26B shows the pullwire handle in a “set” state, in which the guide portion (capture tube 2301) is attached at both ends to the base of the handle 2303. The pullwire 2310 may be inserted from the proximal end of the handle (not shown), threaded through the handle body or base, and into the capture tube, where it passes around and through the capture tube. In FIG. 26C, the distal end of the pullwire reaches a stop or fixing point in the handle, where further advancement of the distal end is prevented. The distal end of the pullwire may be secured in the handle. For example, the distal end may be secured by a gel, adhesive, clamp, or the like, that holds it in place (through it may be withdrawn by the application of appropriate force). As a result of bottoming out at the stop, the force applied to advance the pullwire in the handle may cause the detachable portion of the capture tube 2309 to release from the handle as shown. Further advancement of the pullwire is shown in FIG. 26D; the pullwire 2310 continues to loop out of the handle. The handle may include a lock and a control to release/close the lock to secure the more proximal end of the pullwire in the handle. The pullwire can be removed from the handle by pulling it proximally, until the distal end is withdrawn from the stop and then out of the handle completely, as shown in FIG. 26F. The handle may be re-used by resetting the capture tube. The capture tube can be reset by inserting the end of the capture tube 2309 into the detachable lock in a recess in the handle 2311, as illustrate in FIG. 26G.

The end of the capture tube 2309 may be removably secured in any appropriate manner. For example, the capture tube 2309 may be secured by a friction fit (e.g., a snap fit, etc.), a magnetic connector, or the like. In some variations, the detachable region of the capture tube includes a magnet or a ferrous region that mates with another magnetic (or ferrous region) on the handle body that receives the detachable region.

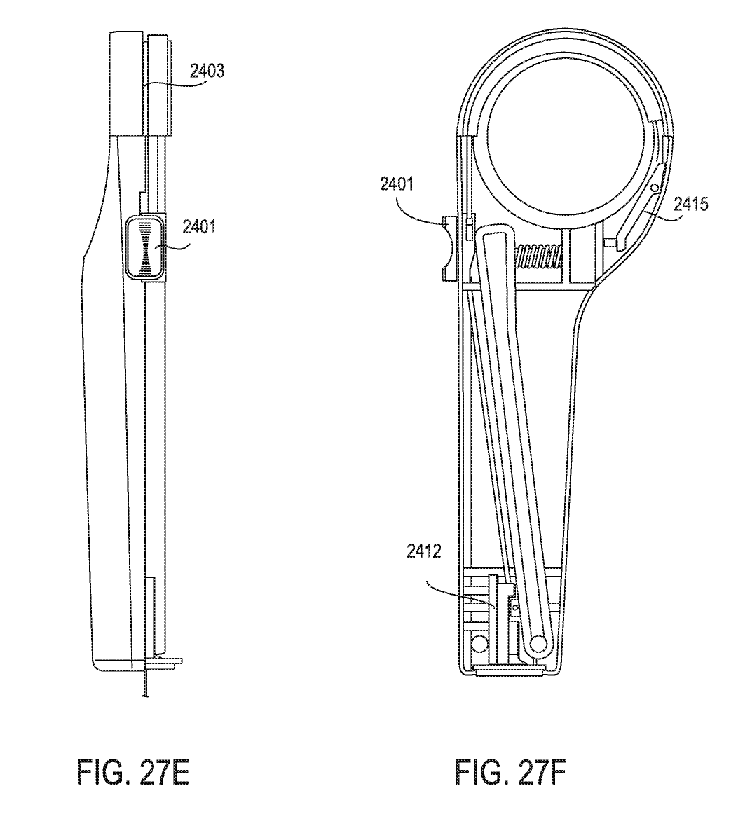

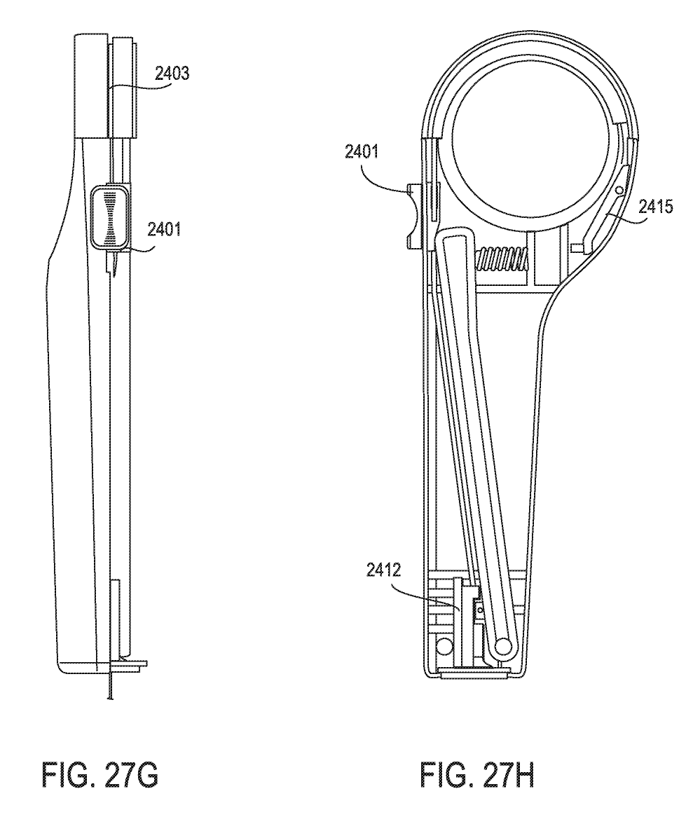

FIG. 27A shows another variation of a distal handle including a window or slot 2403 through which the pullwire may pass after it has been fed into the handle and the distal end of the pullwire has been secured. As the pullwire is fed into the handle, a loop of pullwire may pass out of the window or slot. A control (e.g., slider or button) 2401 may be used to control the opening and closing of the window, and may be used to secure the distal end of the pullwire in the handle, as well as a more proximal end of the pullwire. This is illustrated in FIGS. 27B-27D and again in FIGS. 27E-27H.

For example, in FIGS. 27B-27D, the different positions of the control 2401 are illustrated in this variation. In FIG. 27B the control (slider 2401) is in a first position, a bottom position, corresponding to the closing of the window and setting up of the handle to accept and lock the distal end of the pullwire when it is fed into the handle initially. As before, the pullwire may be passed into the body of the handle, around the closed window region 2405 to a stop on the other side of the looped window region. Once the pullwire reaches the stop, the control may be changed to a second position as illustrated in FIG. 27C. In some variation the handle may detect the pullwire tip at the stop. As before, the pullwire tip may be maintained at the stop by a clamp, adhesive, gel, or the like. When the control is in the second position, the window 2403 opens to allow the wire to bow out of the window so that the loop of wire can expand outwards (similar to the variation shown in FIG. 26E, though without a capture tube. When the control is in position two, the wire may be freely slid within the distal handle. Once the wire is fed into the handle a desired amount, the control may be moved to a third position, locking the proximal end of the wire within the handle body so that tension may be applied to the pullwire by the handle (e.g., when it is used for a decompression, as referred to above). In FIGS. 27B-27D the control is a button or slider, however any appropriate control may be used, including dials, switches, or the like. Also, in some variations the control may integrate the functions described at some of the three positions mentioned above, so that only two positions are necessary (e.g., one for locking/unlocking the distal end and one for locking/unlocking the proximal end.

FIGS. 27E to 27H show cross-sectional views through the variation shown in FIG. 27A during two of the three steps mentioned above for FIGS. 27B-27D. For example, in FIGS. 27E and 27F, the window or slot 2403 is closed, and the button 2401 is in the bottom (first) position. The pullwire may be fed through the device and around the curved window region to engage with the distal lock 2415; a proximal lock 2412 is opened completely so that the pullwire may slide through the proximal portion of the handle. When the slider 2401 is in the second (middle) position (not shown) and the window is opened to allow the wire to bow out of the handle while the distal end of the pullwire is held by the stop (clamp) 2415. The proximal clamp 2412 remains open so that more pullwire can be drawn or pushed into the handle. In FIGS. 27G and 27H, the slider 2401 is in the third (top) position, and the window 2403 is still opened, and a loop of pullwire may be passing out of the window. However, in this position the lower clamp or lock 2412 at the proximal end of the handle is engaged so that the proximal portion of the pullwire in the handle is locked in position.

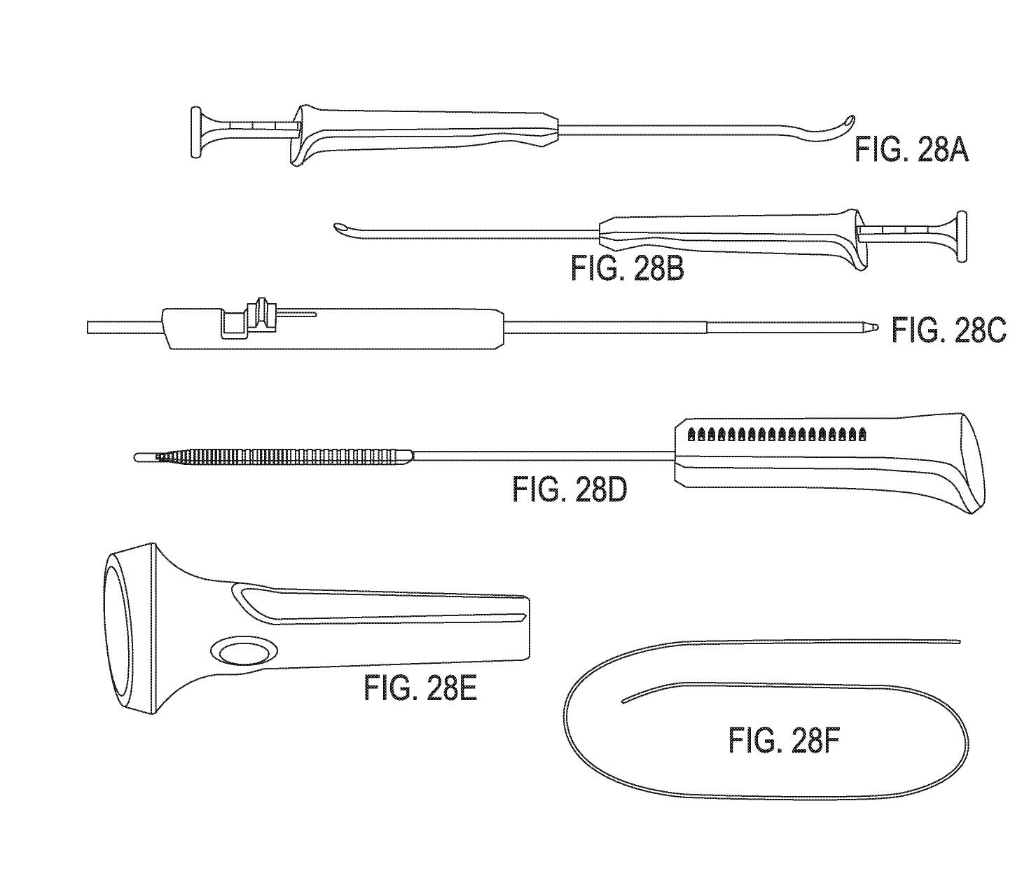

FIGS. 28A-F illustrate a system including various embodiments of the devices described above. Any of the devices (and embodiments of these devices) described herein may be included in a system for treating spinal stenosis. In some variations, more than one type of tool or handle may be included. For example, in the system shown in FIGS. 28A-28F, two variations of probes (28A and 28B) are illustrated.

Another variation of a probe is illustrated in FIG. 29A. In this variation, the probe includes an outer cannula 2601 that is connected to a handle including a proximal grip region 2607. The proximal handle of the probe may be a metal or plastic, or any other suitable material. For example, the handles may be an ABS or Polycarbonate material. The outer cannula is typically stiff, and may be formed of a metal (e.g., stainless steel, Nitinol, etc.), and is curved slightly at the distal end, as mentioned above. An inner member 2603 is slideably disposed within the outer cannula, and is coupled to a plunger or other control 2605 at the proximal end, so that it can be extended and retracted from the distal end of the outer cannula. The inner member 2603 is typically curved so that when extended from the distal end of the outer cannula 2601, it curves, as shown in FIG. 29A. In some embodiments, the inner wall of the outer cannula maybe coated or may comprise a lubricious material to reduce friction between the inner and outer cannula. For example, the inside (or a portion, for example a distal portion of the inside) of the outer cannula may be coated with a polymer coating, such as PARYLENE. The polymer may be deposited via vapor deposition, for example. In some embodiments, the probe may further include a locking mechanism such that the inner cannula remains within the outer cannula in the non-expanded configuration prior to being pushed such that the inner cannula extends from the outer cannula. The locking mechanism may be a wedge or other mechanical lock such that the inner cannula is held in position with friction. In some variations the inner member of the probe is configured so that a distal tip region 2609 is always outside of the outer cannula 2601; in other variations, the inner member is configured to be completely retracted within the outer cannula. The inner member may also be referred to as an inner cannula, since it typically includes a central pullwire lumen 2614, through which a pullwire may be extended distally after the probe has been positioned. The inner member may therefore guide the pullwire through the curved distal end and steer placement of the pullwire.

The inner member (or cannula) shown in FIG. 29A and in cross-section in FIG. 29B is a flattened elongate body including a central pullwire lumen 2614. In general, the pullwire lumen extends all the way from the proximal end of the device (e.g., through the plunger 2605) and out of the distal tip 2609 of the inner member 2603. In this variation, the inner member includes one or more (two support members 2612, 2612′ are shown in FIGS. 29A and 29B) support members that extend along the length of the inner member. The support members are typically solid members, such as wires, rods, or the like. The support members are also typically curved, so that the inner member curves as it is extended distally out of the outer cannula, as shown in FIG. 29A. The support members may be, for example, a wire formed of metal such as stainless steel or a shape memory alloy such as Nitinol. The support member may be positioned within a lumen through the flattened elongate body, as shown in FIG. 29B. In this variation, two support members (though they may be formed using a single wire that extends the length of the device and doubles back around one end to extend back along the length of the device) extend along the longitudinal length of the device. The support members shown in FIGS. 29A and 29B are Nitinol wires that are pre-biased to have a curved shape. The support members extend to either side of the (central) pullwire lumen. In other variations, the support member(s) may extend centrally, and the pullwire lumen may be located to one side. The support members may be sized and configured to retain the distal end and or tip of the inner cannula as described above in reference to the safety retainers.

In some variations the body of the inner member may be formed of a flexible material, such as a polymer, rubber, or the like, that is formed or extruded to have a pullwire lumen, and lumen for the support member(s). For example, the three lumen may be formed in a flat/oval extrusion to form the body of the inner member. In FIG. 29B, the outermost lumen house two Nitinol wires that run down these lumen to either side of the central lumen.

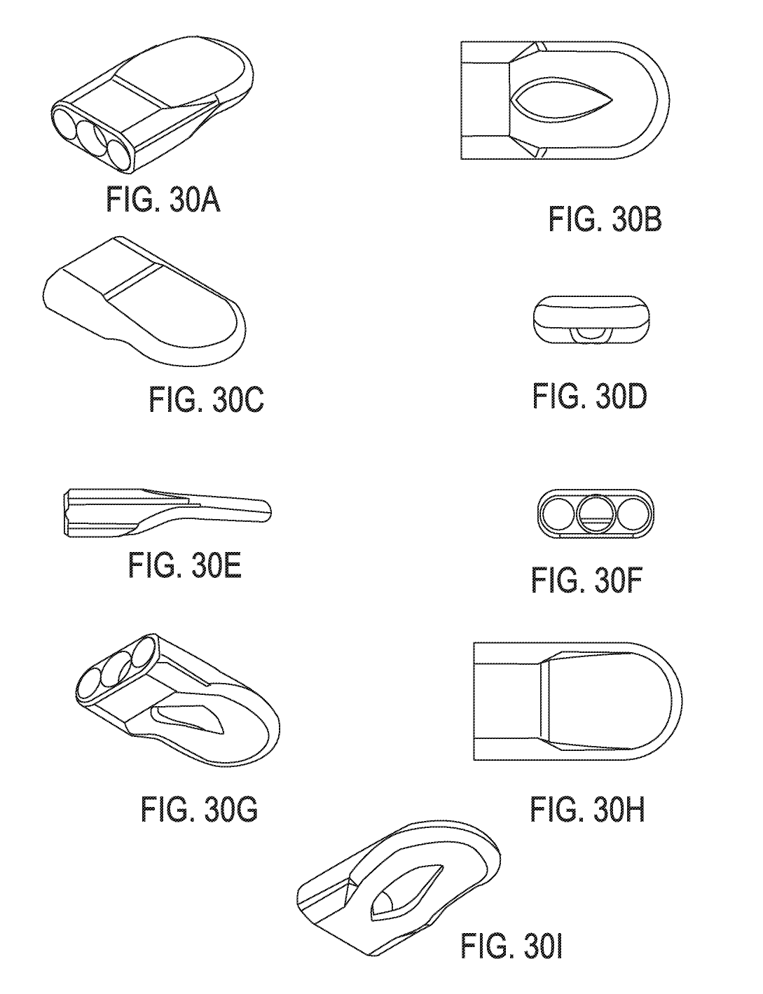

The support wires maybe coupled to the distal tip of the inner member. In general a distal tip may be an atraumatic tip. The distal tip may be flattened and rounded, as indicated in the variation shown in FIGS. 30A-I. In this variation, the distal tip is secured to the distal ends of the two support members (wires 2612, 2612′). For example, the support members may be crimped, clamped, fused, soldered, swaged, etc. within the distal tip shown. FIGS. 30A-I illustrate different top perspective views (FIGS. 30A, H, C), front (FIG. 30D), side (FIG. 30E), and back (FIG. 30F) views, and bottom perspective views (FIGS. 30G, B, I). The distal tip may also be coupled to the inner cannula. The distal tip may be coupled by way of crimping, fusing, welding, swaging, etc., or any combination thereof. For example, the distal tip may be mechanically coupled to the end of the inner cannula via a ring swaged onto the end of the catheter. A polymer, such as PEEBAX, may then be fused over the mechanical ring.

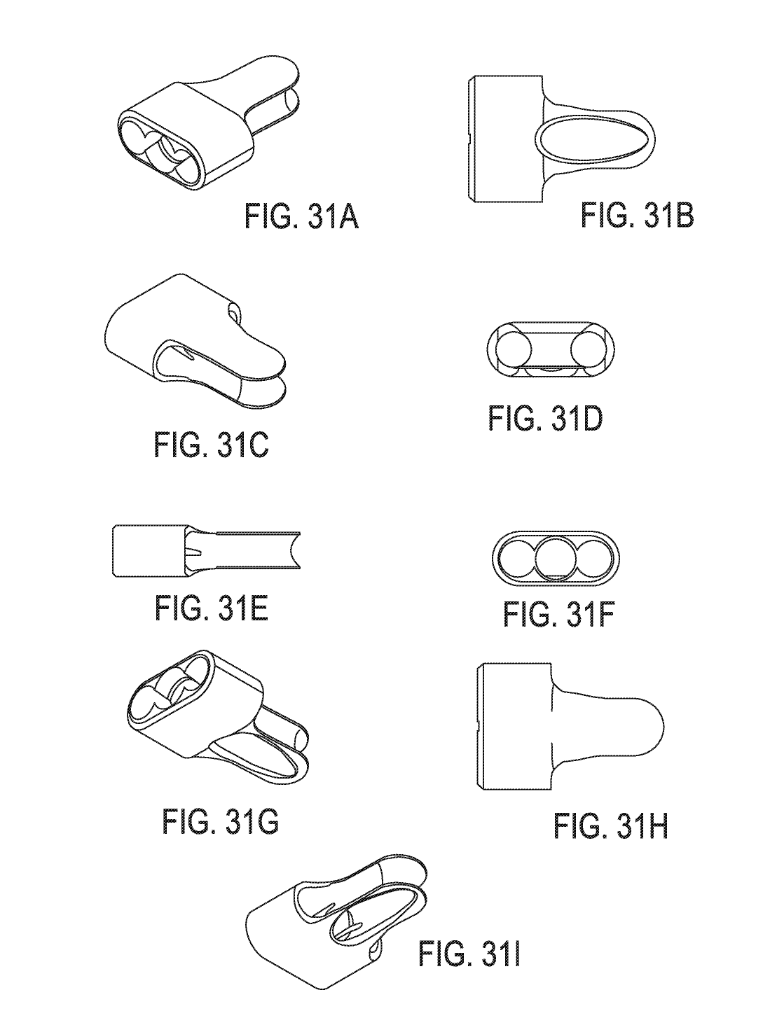

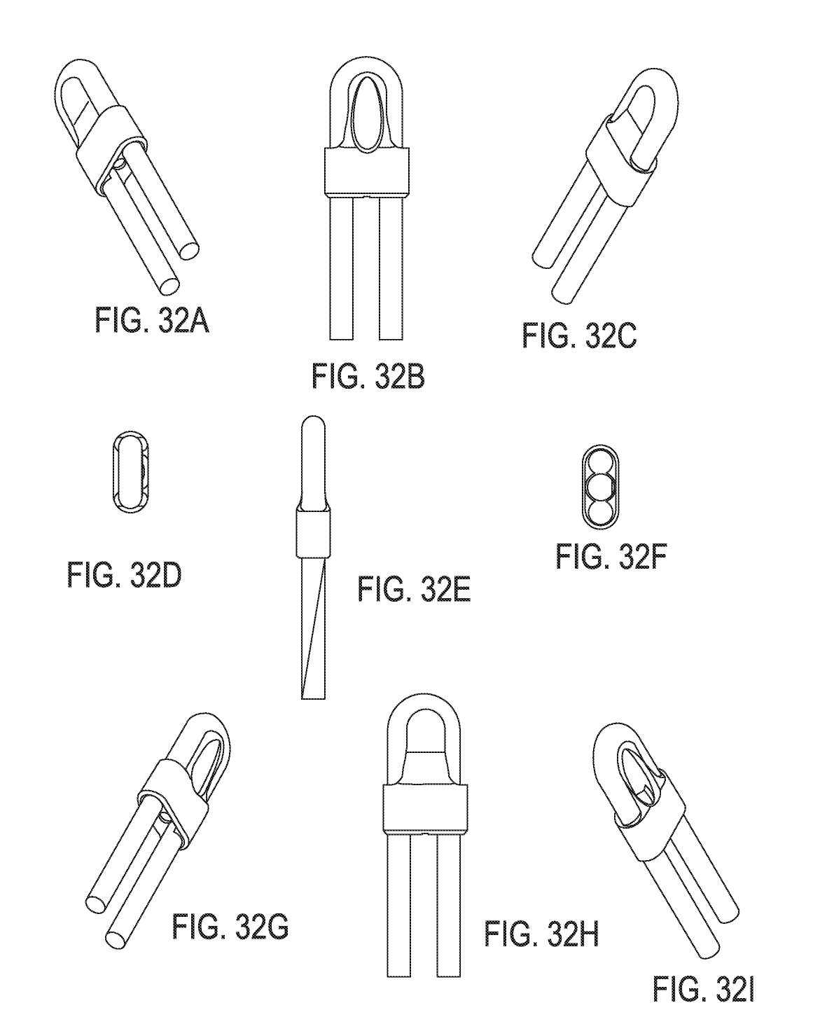

FIGS. 31A-32I illustrate another variation of a distal tip for the inner member of a probe. In this variation, the support member(s) loop or wrap around the front of the tip. Thus, the tip may be slightly narrower than the variation shown in FIGS. 30A-I. For example, FIGS. 31A-I illustrate different top perspective views (FIGS. 31A, H, C), front (FIG. 31D), side (FIG. 31E) and back (31F) views, and bottom perspective views (FIGS. 31G, B, I) of this variation. FIGS. 32A-I show the same variation of FIGS. 31A-I with the support member (cable, wire, rod, etc.) indicated.

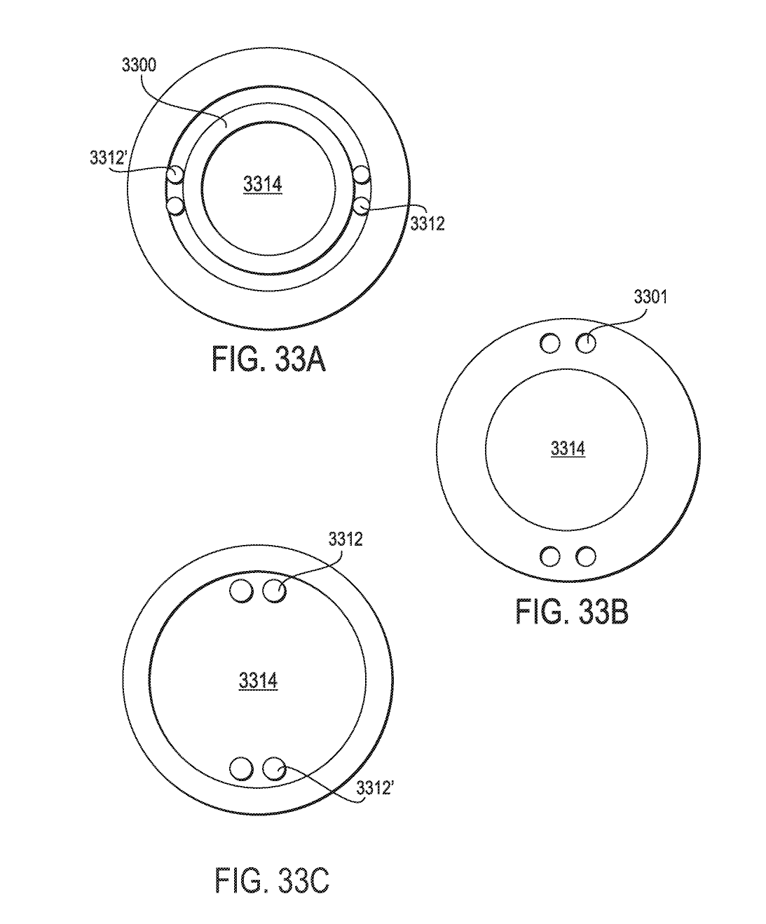

The inner member (or cannula) shown in cross-section in FIG. 33A is an elongate body including a central pullwire lumen 3314. In general, the pullwire lumen extends all the way from the proximal end of the device (e.g., through the plunger) and out of the distal tip of the inner member. In this variation, the inner member includes one or more support members 3312′ and 3312′ that extend along the length of the inner member. As shown, the elongate body comprises a single lumen 3314 with the tubular body 3300 and support members 3312 and 3312′ longitudinally disposed within the elongate body. The tubular body and the support members may be free floating or fixed within the elongate body. As shown in FIG. 33B, the elongate body comprises a central pullwire lumen 3314 and at least one support member lumen 3301. The tubular body may be disposed within the pullwire lumen and the support members may be disposed within the support member lumens. As shown in FIG. 33C, in some embodiments, the pullwire may run freely within the elongate body, rather than through a tubular body.

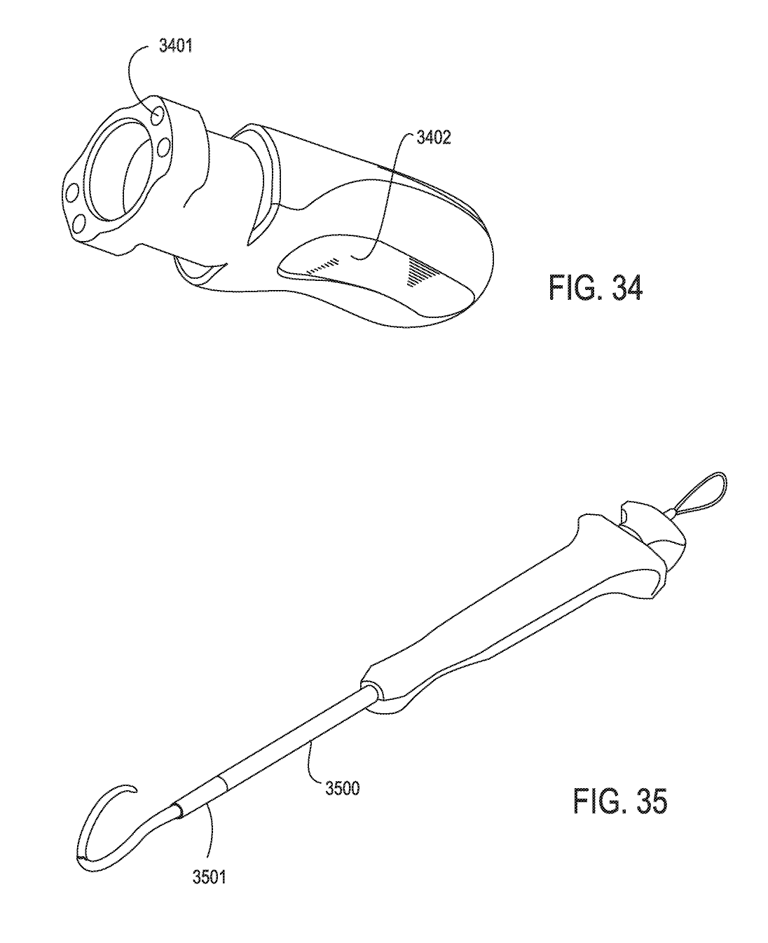

As shown in FIG. 34, a distal tip may comprise at least one support member lumen 3401 configured to receive the support member. The distal tip may further comprise a port 3402, through which a pullwire may exit through the probe.

As shown in FIG. 35, in some embodiments, a probe may further include a stiffening tube 3500. This stiffening tube may be positioned around the outer cannula, or alternatively the stiffening tube may replace the outer cannula. The stiffening tube may include a tapered portion 3501. The reduced diameter of the tapered portion may allow for better visualization of the distal end of the probe and/or the adjacent tissue.

The devices, systems and methods described herein may be use in any appropriate surgical procedure, particularly for the surgical treatment of spinal stenosis. For example, described herein are systems including one or more of the following devices: a guidewire, a probe for positioning a guidewire, and a tissue modification device for use with the guidewire. As described herein, the systems and methods may be used to decompress one or more spinal regions. In particular, any of these devices may be used to decompress nerve roots within the spinal anatomy along various paths. Because these devices are flexible, and may be appropriately sized and shaped to fit within a neural foramen, these devices may be used to accesses appropriate regions of the spine from a single access point (e.g., from the patient's midline or near-midline region). While the systems and methods described herein may used to decompress one or more spinal regions they may also be configured to avoid portions of the neural anatomy or other non target tissue within the spinal regions that are being decompressed.

As described herein, devices, systems and methods may be configured to avoid the non-target tissue (including neural anatomy) exiting through the neural foramen in one of several variations, as described in detail below. For example, a probe and guidewire may be configured and/or the method of use may be configured such that the guidewire is positioned to avoid the non-target tissue (including neural anatomy).

In general, the procedure may be used to decompress spinal nerve roots on the unilateral or contralateral side from the access point. A probe or guide may be introduced into the spinal epidural space (or along or just within the ligamentum flavum) at an appropriate spinal level. In some embodiments, the probe may be introduced using image guidance (such as fluoroscopy) and/or tracking (e.g., electromagnetic tracking). Introduction may be either via percutaneous puncture, open laminotomy, mini-open, tube-based, etc.

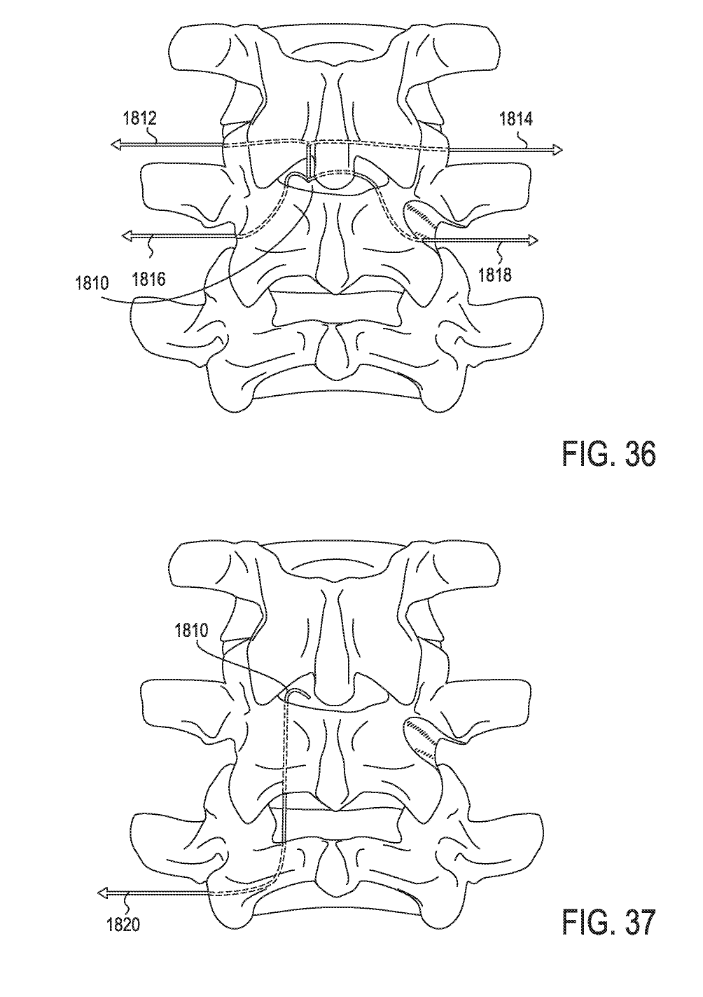

As shown in FIG. 36, the device may be used to decompress an ipsilateral or contralateral proximal nerve (in a lateral recess). A guide or probe may be deployed immediately cephalad to the caudal segment pedicle on the appropriate side (e.g., location 1810). This access point can be confirmed radiographically. If neural structures adjacent to the guide cannot be directly visualized, the relationship of these structures to the guide or tissue modification devices can be determined using electrical stimulation, ultrasound imaging, endoscopic mean or other techniques.

As shown in FIG. 36, the guidewire may be threaded along a path from location 1810 to where it exits through the foramen, as shown by at least one of arrows 1812 (for ipsilateral decompression of the nerve root origin at the disk level) and 1814 (for contralateral decompression of the nerve root origin at the disk level). Alternatively, as shown in FIG. 36, the guidewire may be threaded along a path from location 1810 to where it exits through the foramen, as shown by at least one of arrows 1816 (for ipsilateral decompression along the nerve root) and 1818 (for contralateral decompression along the nerve root). In some embodiments, the probe/guide is removed once the guidewire has been positioned.

The guidewire may include a wire exchange tip on its proximal end, as described in more detail below. A flexible tissue modification device (as described in more detail below) is attached to the proximal wire exchange tip, and a distal handle may be secured to guidewire at the distal wire tip. The device can then be introduced into the epidural space and then into the lateral recess by careful upward force applied to the distal handle. In some embodiments, the device is pulled by the guidewire on the path through the spinal anatomy. As described above, suitable paths include paths shown by arrows 1812 and 1814 and/or 1816 and 1818 to decompress the nerve root origin at disk level and/or along the nerve root, respectively.

Once the device is in place as confirmed visually or radiographically, bimanual reciprocating strokes may be utilized to decompress dorsal impinging bone or soft tissue at the nerve root origin. In some embodiments, the proximal handle of the tissue modification device and the distal handle may be held a distance apart while moving the tissue modification device to remove tissue, such that the tissue modification device is positioned and moved to avoid the non-target tissue (including neural anatomy).

The probe/guide may be reinserted to decompress the ipsilateral or contralateral distal (foraminal) portion of the nerve root, so that the same (or a different) tissue modification device may be used to decompress another region of the spine (or nerve root) using the same access or entry site. As shown in FIG. 37, the devices described herein can used to decompress the ipsilateral or contralateral (not shown), or both, regions adjacent the level proximal to the nerve root (lateral recess). A guide may be deployed in the same access point (location 1810) as described above. As shown in FIG. 37, the guidewire can then be threaded along a path from location 210 to where it exits through the foramen, as shown by arrow 220 (for ipsilateral decompression of the adjacent nerve root origin).

Described herein are systems including one or more of the following devices: a guidewire and a probe for positioning a guidewire. The guidewire is typically long (e.g., elongated) and flexible, and may have a sharp (tissue penetrating) distal end and a proximal end that allows it to be coupled to a guidewire coupling member securely. For example, the guidewire includes a ball or other shaped end (which may be conical, tubular, ring, etc.) secured to the distal end for coupling to a guidewire coupling member. The proximal end may be configured to lock into a guidewire coupling member at the distal end of a tissue modification device. Similarly, the proximal end of the guidewire may be configured to pass through the probe so that the probe may be removed from over the proximal end of the guidewire during operation.

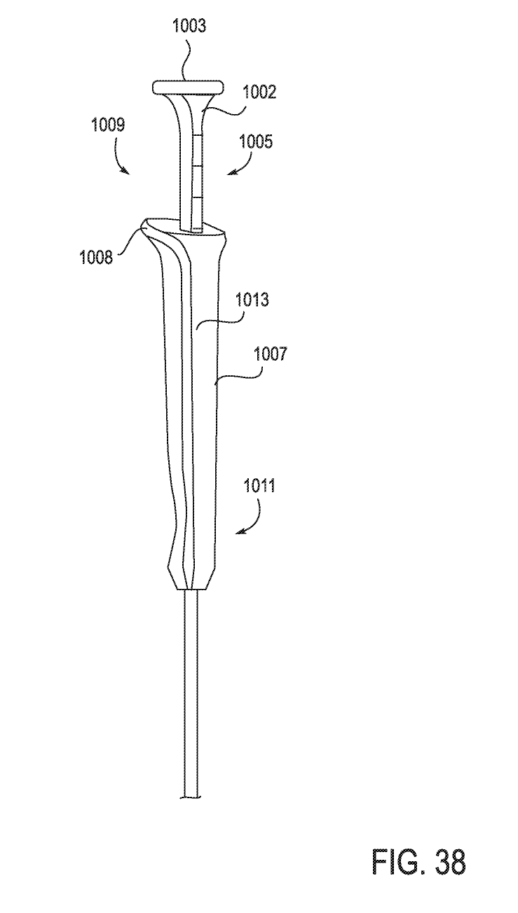

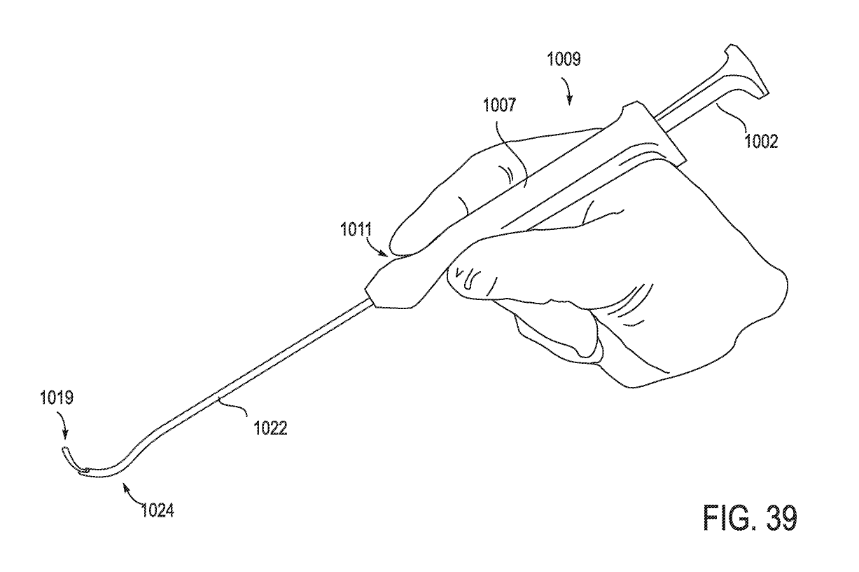

For example, in the variation illustrated in FIGS. 38 and 39, the probe 1009 includes a handle portion having a pusher or plunger 1002 that communicates with an internal cannula slideably disposed within the external cannula so that the internal cannula may be extended from the distal end of the probe for placement around a target tissue, as illustrated in FIG. 39. The pusher includes a flanged 1003 proximal end having a finger (e.g., thumb) pushing surface that is perpendicular to the long axis of the device (including the long axis of the handle). As described in greater detail, this proximal end may be formed to more readily allow insertion of a guidewire by guiding the guidewire into the lumen of the inner cannula. The pusher is calibrated 1005 along the side in a top-facing surface. The calibration shown in this example includes markings to indicate depth (e.g., how far down the pusher has been extended), which corresponds to how far out of the distal end the inner cannula is extended. The calibrations may include alphanumeric symbols, colors, textures, or any combination of the like. The calibrations may be referenced to distance (e.g., depth, length, etc.), or they may be un-referenced (as shown in FIG. 3).

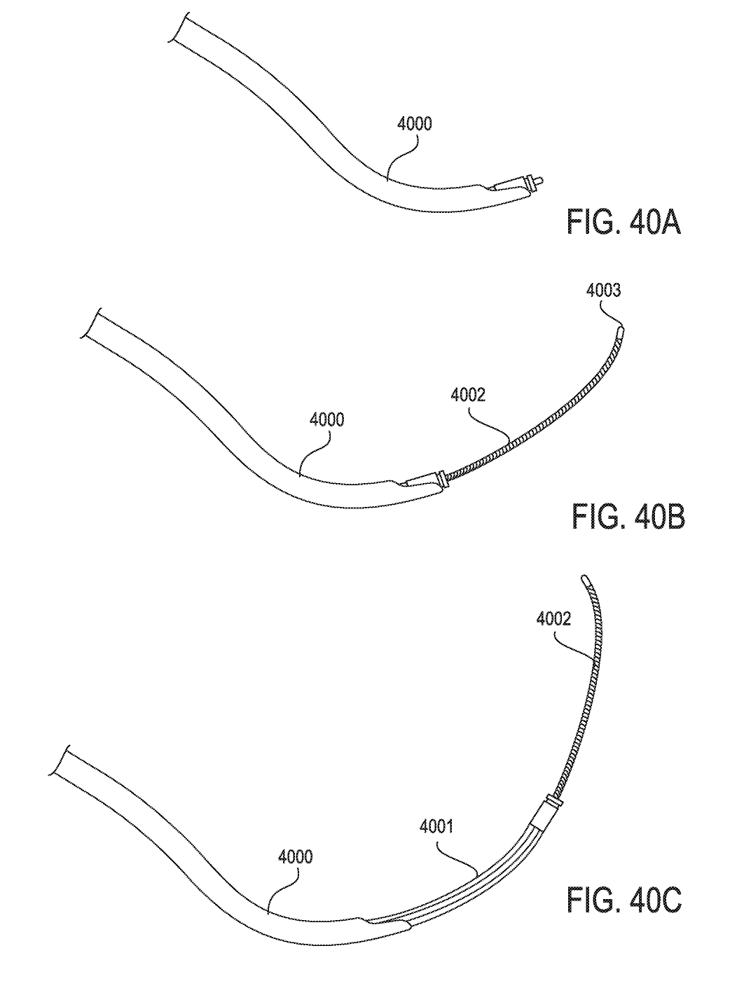

In an alternative variation, as shown in FIGS. 40A to 40C, the probe may include an outer cannula 4000, an inner cannula 4001, and a stylet or guidewire 4002. As described above, the outer cannula may be positioned within the spine of a patient. In some embodiments, the outer cannula may be positioned while the inner cannula and the guidewire are disposed (e.g. retracted) within the outer cannula, as shown in FIG. 40A. Once the outer cannula is in position, the guidewire may be deployed from the distal end of the outer cannula, as shown in FIG. 40B. As shown, the guidewire is thin and by having a smaller cross sectional area may be more easily deployed within the anatomy of the spine. As shown the guidewire may have an atraumatic distal tip 4003 such that as the guidewire is deployed it will not damage the surrounding tissue such as the neural or vascular tissue (i.e. non-target tissue). Once the guidewire is deployed, the inner cannula, or catheter, may be deployed over the guidewire. As shown in FIG. 40C, as the inner cannula is deployed from the distal end of the outer cannula, the guidewire may function to guide the inner cannula through the spinal anatomy. Once the inner and outer cannulas are in position, the stylet or guidewire may be removed, and a second guidewire or pullwire (for example, with a sharp distal tip) may be thread through the inner cannula and into the spine of the patient. Alternatively, the stylet or guidewire may be advanced further distally from the probe such that it exits the patient.

In general, the distal end of the probes described herein may be curved or bent, and/or may be curvable or bendable. The outer distal end may be more or less curved. The inner cannula may be configured to bend as it exits the distal end of the outer cannula, as shown, thereby increasing the ability of the probe to guide a guidewire around a target tissue.

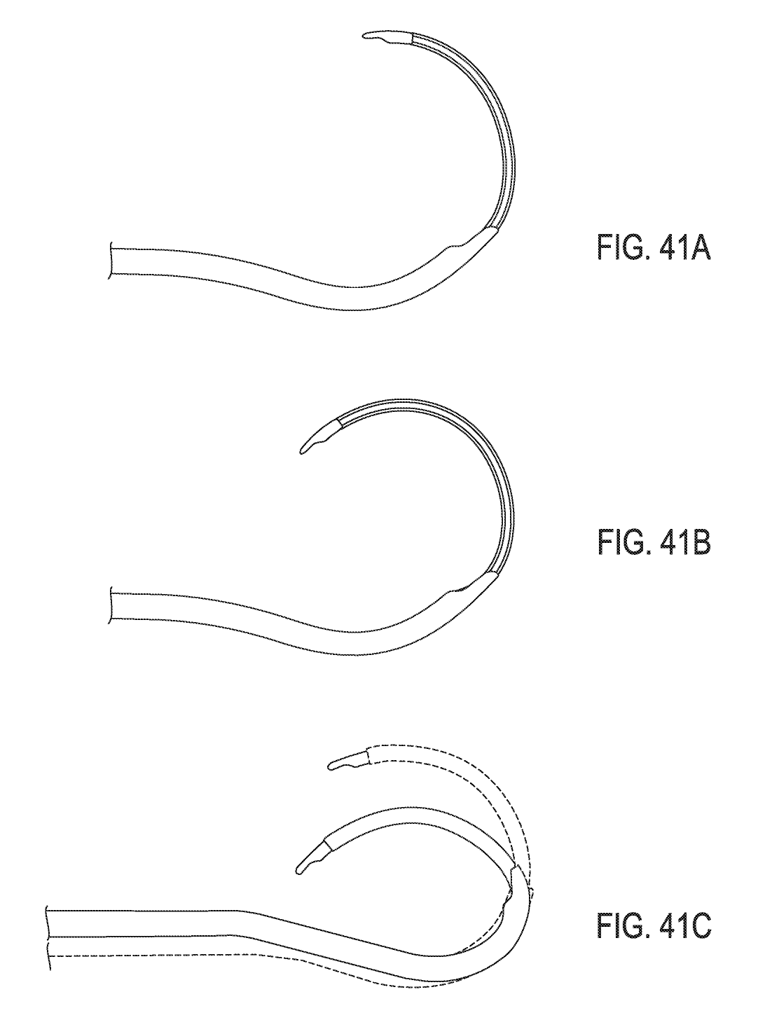

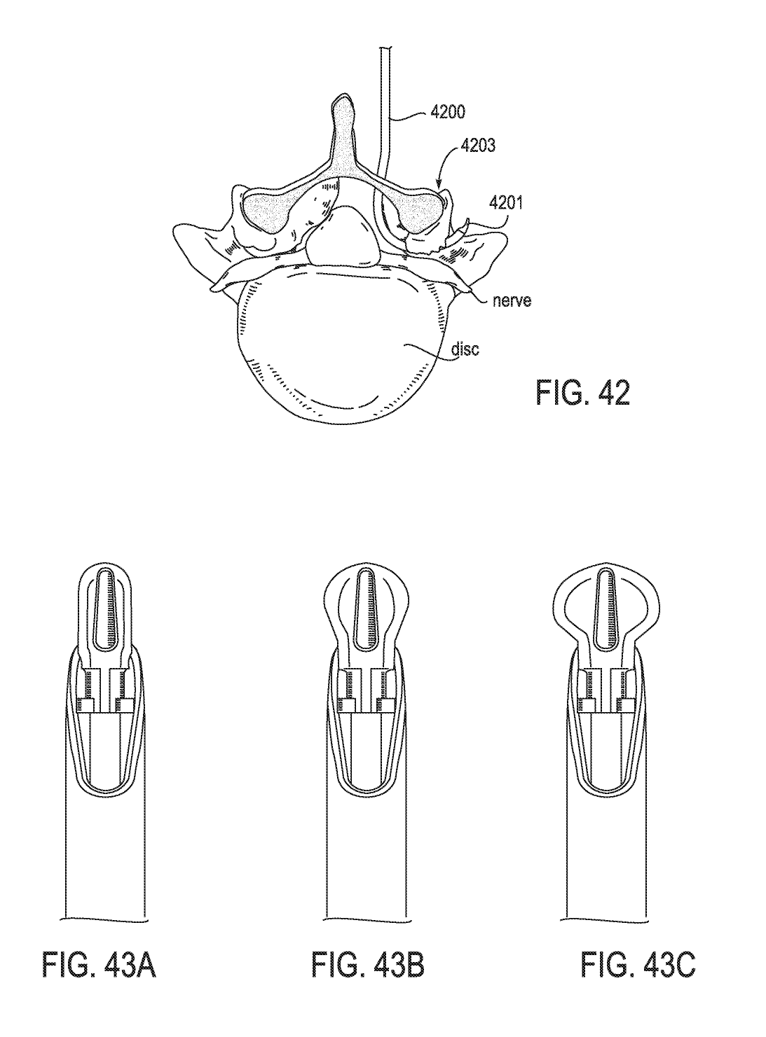

As shown in FIGS. 41A to 41C, the probe may include an outer cannula and an inner cannula, slidable within the outer cannula to an extended position (as shown). As shown, both the inner and outer cannulas may be curved at their distal end. Alternatively, the outer cannula 4200 may be straight, and the inner cannula 4201 may be curved as it extends from the outer cannula. The cannulas may be curved at the distal end such that they can be fed around a facet joint 4202 and into a neural foramen of a spine, as shown in FIG. 42. As shown in FIGS. 41A-41C, the inner and outer cannulas may be configured in one of several configurations. For example, the probe illustrated in FIG. 41B may have a smaller radius curve (i.e. tighter curve) than the probe illustrated in FIG. 41A. As shown in FIG. 41C, and described in detail below, in some embodiments, the inner cannula of the probe illustrated in FIG. 41A may interfere with a nerve as it is extended from the outer cannula. However, the inner cannula of the probe illustrated in FIG. 41B may stay inside or above (or posterior to) the nerve as it is extended from the outer cannula.

For example, some configurations and probe shapes may be more suitable for avoiding non-target tissue, such as neural tissue. The “standard” probe (illustrated in FIG. 41A) may be advanced below (or anterior to), pierce, or otherwise irritate the nerve structure as the outer cannula is placed within the spine and/or as the inner cannula is extended from the outer cannula. Alternatively, the “tight” probe (illustrated in FIG. 41B) may remain posterior to the nerve and therefore may not pierce or otherwise irritate the nerve structure as the outer cannula is placed within the spine and/or as the inner cannula is extended from the outer cannula. The geometry and configuration of the “tight” probe may allow the inner and/or outer cannula to remain posterior to the nerve and therefore not irritate this sensitive tissue.

The “tight” probe (illustrated in FIG. 41B), may wrap tighter around the facet joint and remains dorsal (or posterior) to the nerve root. As shown, however, the “standard” probe (illustrated in FIG. 41A) is more anterior and closer to the nerve.

In some embodiments, as described above, probes may have different tip configurations. The distal tips are preferably designed to be large enough such that they are not sharp and do not easily pierce neural tissue. However, the probe tips cannot be so large that they cannot be positioned into and through a neural foramen. In some embodiments, as shown in FIGS. 43A to 43C, the probe may have a wider “duck-billed” configuration (43C), while the distal tip of the probe as shown in FIG. 43A has a more narrow rounded tip.

Although much of the description and accompanying figures generally focuses on surgical procedures in spine, in alternative embodiments, devices, systems and methods of the present invention may be used in any of a number of other anatomical locations in a patient's body. For example, in some embodiments, the flexible tissue modification devices of the present invention may be used in minimally invasive procedures in the shoulder, elbow, wrist, hand, hip, knee, foot, ankle, other joints, or other anatomical locations in the body. Similarly, although some embodiments may be used to remove or otherwise modify ligamentum flavum and/or bone in a spine to treat spinal stenosis, in alternative embodiments, other tissues may be modified to treat any of a number of other conditions. For example, in various embodiments, treated tissues may include but are not limited to ligament, tendon, bone, tumor, cyst, cartilage, scar, osteophyte, inflammatory tissue and the like. Non-target tissues may include neural tissue and/or neurovascular tissue in some embodiments or any of a number of other tissues and/or structures in other embodiments. In one alternative embodiment, for example, a flexible tissue modification device may be used to incise a transverse carpal ligament in a wrist while inhibiting damage to the median nerve, to perform a minimally invasive carpal tunnel release procedure. Thus, various embodiments described herein may be used to modify any of a number of different tissues, in any of a number of anatomical locations in the body, to treat any of a number of different conditions.

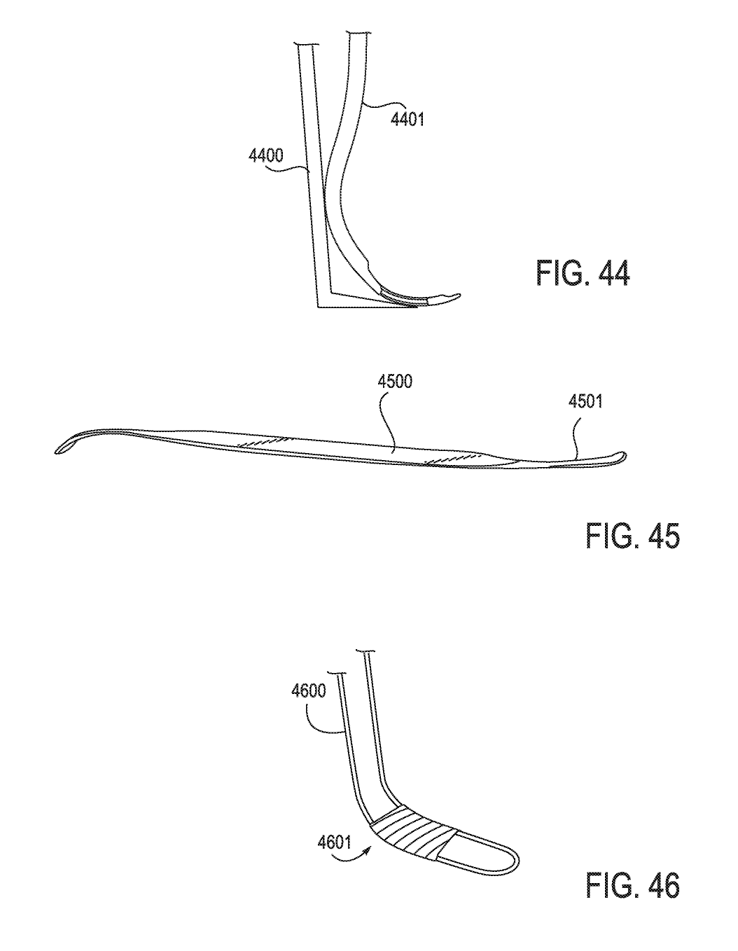

In some embodiments, it may be beneficial to push the disc out of the way such that the probe may be deployed into the spine. A conventional Woodson probe may be used in spinal procedures. For example, a Woodson probe may be used to probe or palpate a pedicle from within a canal to assist in pedicle screw placement. As shown in the FIG. 44, a modified Woodson-type probe 4400 device may utilized to push down the disc and then to receive the probe 4401 while it holds the disc down and out of the way. For example, the modified probe may function as a “shoe horn” to increase the ability to access the spine with the access probe as described above.

This “shoe horn” device could also include a roughened portion on the bottom of the device, i.e. the side of the device adjacent to the disc, to aid in gripping onto the disc and pushing the disc out of the way. The device may also include suction.

As shown in FIG. 45, a probe device 4500 may be used instead of or in conjunction with a modified Woodson probe 4400, in order to access a spine of a patient. In some embodiments, the probe instrument 4500 may be inserted between an interlaminar window and toward a spinal disc of a patient. The distal end of the probe may then be advanced into a neural foramen and onto the spinal disc, as described above, such that the disc material is pushed down (anteriorly) and out of the way of the neural foramen. The paddle portion 4501 may then be rotated such that the distal end of the probe instrument is rotated against a first and second pedicle of the neural foramen such that the first and second pedicles are distracted from one another. One, two or more probe instruments may be rotated to distract the spine. In some embodiments, once the spine has been access and distracted, the distal end of the probe may be locked into position. As described above, an access probe may then be advanced into the spine and the area created by the probe instrument(s).

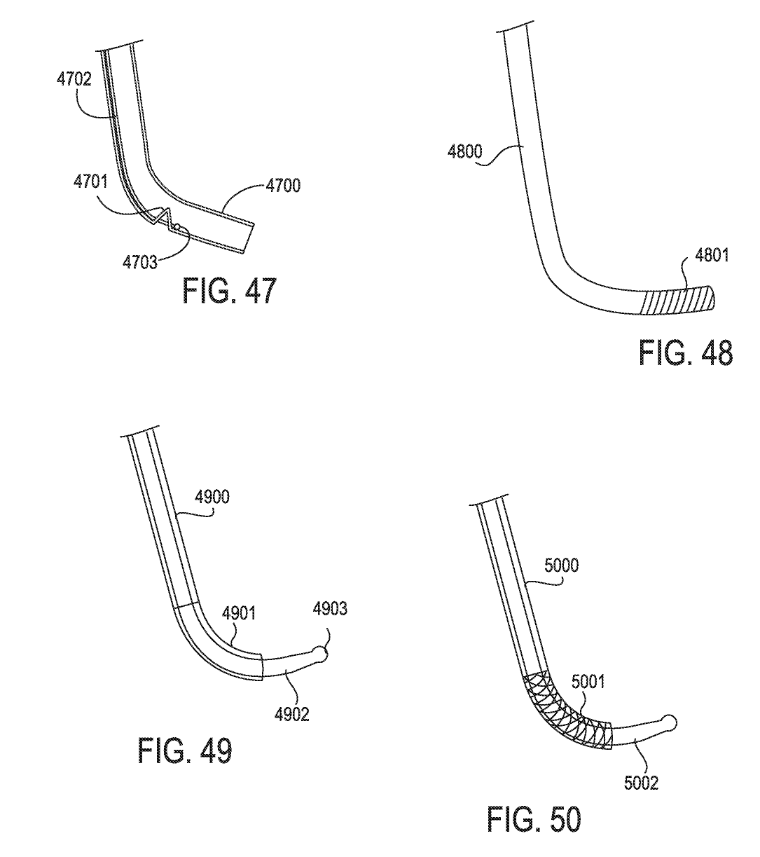

As described above, the access instrument may include a curved distal portion to guide a guidewire around the target tissue and out of the spine. In some embodiments, it may be difficult to remove the curved portion of the access device from the spine once deployed due to the anatomy of the spine and the degree of stenosis or disease of the spine. As shown in FIGS. 46-50, the outer cannula is configured to allow the curved distal portion of the outer cannula to bend or flex as it is removed from the spine. Preferably the outer cannula is rigid enough such that it may be pushed into the spine, especially into a tight diseased portion of the spine, while at the same time flexible enough to be removed easily. As shown in FIG. 46 the outer cannula 4600 may include a flexible element 4601 at the curved portion of the cannula. The flexible element may be a different material, such as plastic, or may include slits or slots to allow the cannula to bend. As shown in FIG. 47, the outer cannula 4700 may include a hinge point 4701. The cannula may include a pull wire 4702 coupled to the cannula at point 4703, beyond the hinge point, such that when the pull wire is pulled, the device will straighten out and allow for easier removal. As shown in FIG. 48, the distal end of the cannula 4800 may include a flexible coil tip 4801. As shown in FIG. 49, the distal curved portion of the outer cannula 4900 may comprise a flexible material 4901. For example, the distal portion may be made out of a PEEK material or any other suitable material. As shown, the inner cannula 4902 having a distal tip 4903 extends from the flexible distal end of the cannula. As shown in FIG. 50, the curved distal portion of the outer cannula 5000 may comprise a mesh or frame 5001 that is flexible. In some embodiments the mesh or frame may be a flexible material such as plastic or NITINOL.

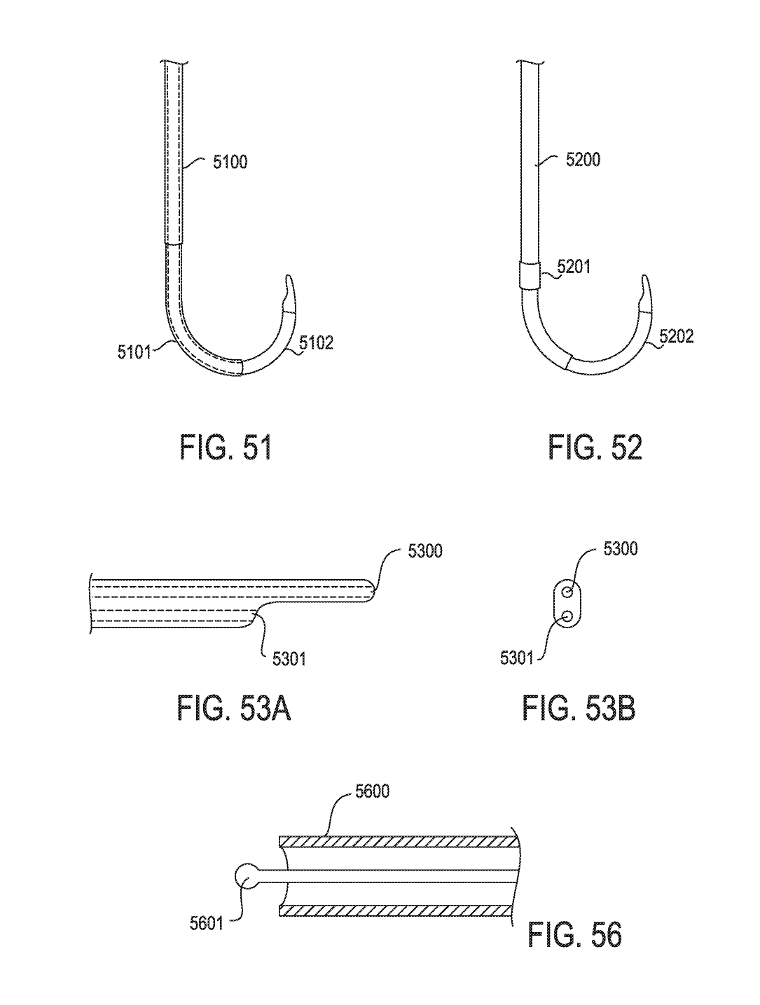

Alternatively, in some embodiments, as shown in FIGS. 51 and 52, the outer cannula may include a mechanism to allow for easy removal of the device. As shown in FIG. 51, outer cannula 5100 may be telescoping. For example, the outer cannula may include a first component that is relatively straight and relatively rigid. This portion may be used to access the spine initially. Once the outer cannula is in place, an inner cannula 5101 may be deployed from the outer cannula. The inner cannula may be rigid or flexible. In some embodiments, as shown in the inner cannula may include a distal curved portion. The probe of this embodiment may also include a third telescoping element, the inner catheter 5202. The inner catheter may be deployed such that it curves around the target tissue, such as the facet joint for example. A wire may be deployed through the inner catheter and into the spine. Once the wire is positioned with the probe, the inner catheter may be retracted into the inner cannula. Then, the inner cannula may be retracted into the outer cannula. Once inside the outer cannula, the straight outer cannula may be easily pulled proximally out of the spine. In some alternative embodiments, not shown, the curved portion(s) of the access device may be rotatable with respect to one another, such that they can be rotated and moved into a substantially straight cannula which is easily removable from the spine.

Alternatively, as shown in FIG. 52, the access instrument may include a clutch or release mechanism 5201. The mechanism may release the curved distal portion from the proximal portion of the cannula 5200 such that the access instrument can be removed. Alternatively, the clutch or release mechanism may prevent the removal of the access device until the inner cannula and/or catheter 5202 is properly stored within the outer cannula.

In some embodiments, the neural foramen of the spine to be accessed may be very small, tight, and/or diseased, and may therefore be very difficult to access with an access device. As described herein, an access device may include an outer cannula and an inner catheter. The inner catheter may include a NITINOL center encased in a plastic extrusion. The inner catheter may also include a distal tip coupled to the NITINOL and/or the plastic extrusion. In some embodiments, it may be desirable or beneficial to reduce the outer diameter of the inner catheter and/or the distal tip to aid in access in the spine. FIGS. 53A to 58B illustrate alternative variations of an access device configured to aid in accessing the spine with the access or probe device.

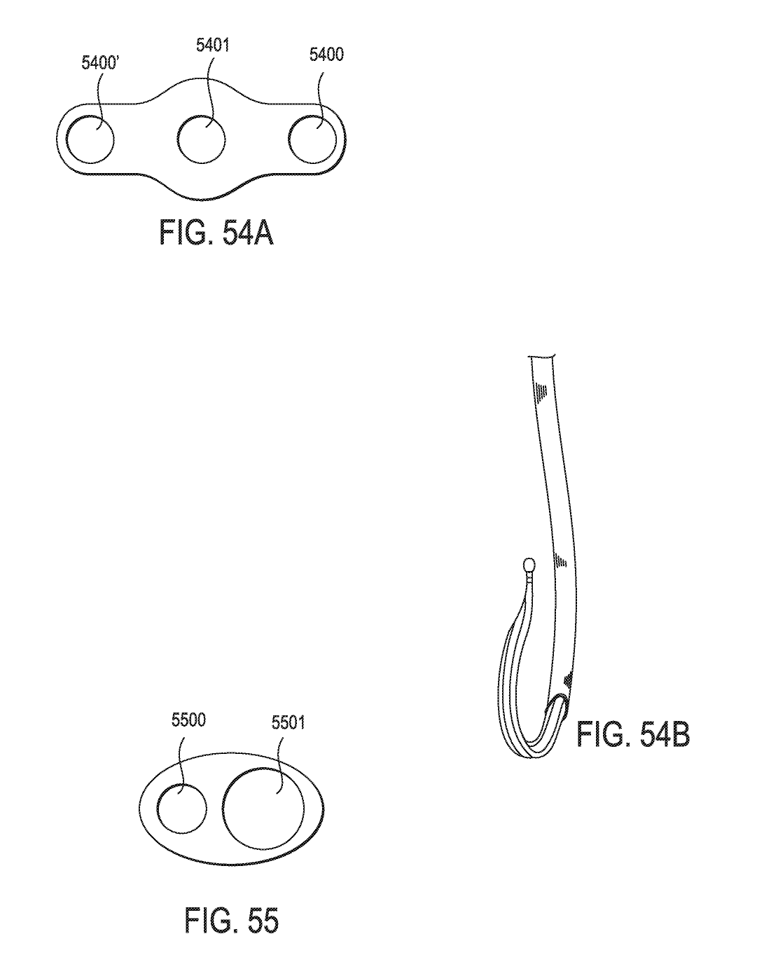

As shown in FIGS. 53A and 53B, the inner cannula or catheter may include two lumens 5300 and 5301. As shown, a first lumen 5300 may be configured to receive a stiffening or support element. The stiffening element may aid in shaping the inner catheter (e.g. around the target tissue) and or add strength to the inner catheter. The stiffening element may also function to couple to the distal tip in the event of a failure or breakage of the catheter. The second lumen 5301 may be configured to receive a pullwire. The pullwire will be deployed through the inner cannula or catheter of the probe device, around the target tissue, and out of the spine. As shown, the distal end of the catheter may have a taper such that the stiffener portion of the inner catheter may extend further than the wire portion. This may allow a smaller diameter to first provide access to the spine and then guide a larger diameter into location. As shown in FIGS. 54A to 55, the catheter may have alternative configurations. For example, as shown in FIGS. 54A to 54B, the catheter may include three lumens. As shown in FIG. 54A, the elongate body of the catheter may include a central lumen 5401 to receive the pullwire, and peripheral lumens 5400 and 5400′ configured to receive support/stiffening members. As shown in FIG. 54B, the distal end of the elongate body may be tapered. FIG. 55 illustrates an alternative embodiment with a pullwire lumen 5501 and a support member lumen 5500.

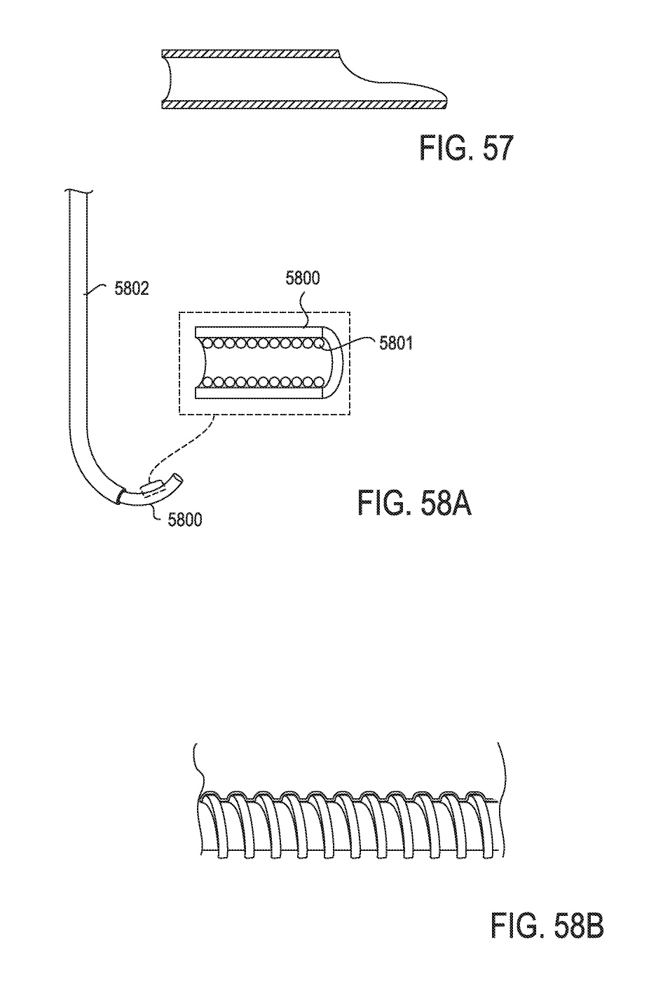

As described above, the inner catheter may include a NITINOL center encased in a plastic extrusion. In some embodiments, as shown in FIG. 56, the NITINOL center may be removed from the catheter. The plastic portion of the catheter 5600 may then be reduced in diameter. In some embodiments, as shown, it may desirable to include a NITINOL or other material stylet 5601 to be placed within the plastic catheter. In some embodiments, it may be desirable to metalize the inner diameter of the plastic catheter to prevent the distal tip of the guidewire from catching on the inner diameter of the plastic catheter. In some embodiments, as shown in FIG. 57, the plastic catheter may not include a distal tip. Alternatively, as shown, the plastic, such as PEEK, may be shaped into a tip configured to access a spinal foramen for example. In some embodiments, the distal end of the catheter may be shaped to form a ball tip. Alternatively, the plastic casing may be removed from the catheter, and the catheter may comprise only the NITINOL center.

Alternatively, as shown in FIG. 58A, the catheter 5800 may comprise a coil 5801. The coil may be stainless steel, NITINOL, or any other suitable material. The coil may be a single coil or may alternatively be a counter rotating coil. In some embodiments, as shown in FIG. 58B, the catheter may comprise a flexible metallic conduit. For example, as shown, in FIG. 58B, the catheter may be a square locked tube. In some embodiments, the coil may include an outer sheath to help maintain the shape. The sheath may be PPEK, NYLON, PEBAX, or any other suitable material. In any of the embodiments described above, the outer cannula 5802, inner cannula, and/or inner catheter 5800 may include a lubricant in their inner diameter to reduce friction. Alternatively, the inside diameter of the outer cannula and the outside diameter of the inner cannula may be coated in a lubricious material such as TEFLON or PARYLENE.

In some embodiments, access to the spine may be difficult to achieve due to a patient's specific anatomy. For example, at the level of the spine to be treated with the system described herein, the patient may have a bulging disc, a disc herniation, a collapsed disc (reducing the pedicle to pedicle height), and/or spondylolisthesis (i.e. a forward dislocation of one vertebra over the one beneath it). Each of these anatomical abnormalities may increase the difficulty of accessing the epidural space and neural foramen with the access tools (e.g. access probe and guidewire) described above. In some embodiments, positioning the patient such that their spine is in flexion may open up the interlaminar window and/or neural foramen, thus improving the ability to access the spine with the access tools. In some embodiments, the operating table may be adjusted to place the spine in flexion. For example a Jackson Table may be used with a Wilson Frame. The Wilson Frame may be adjusted to put the patient's spine in flexion.

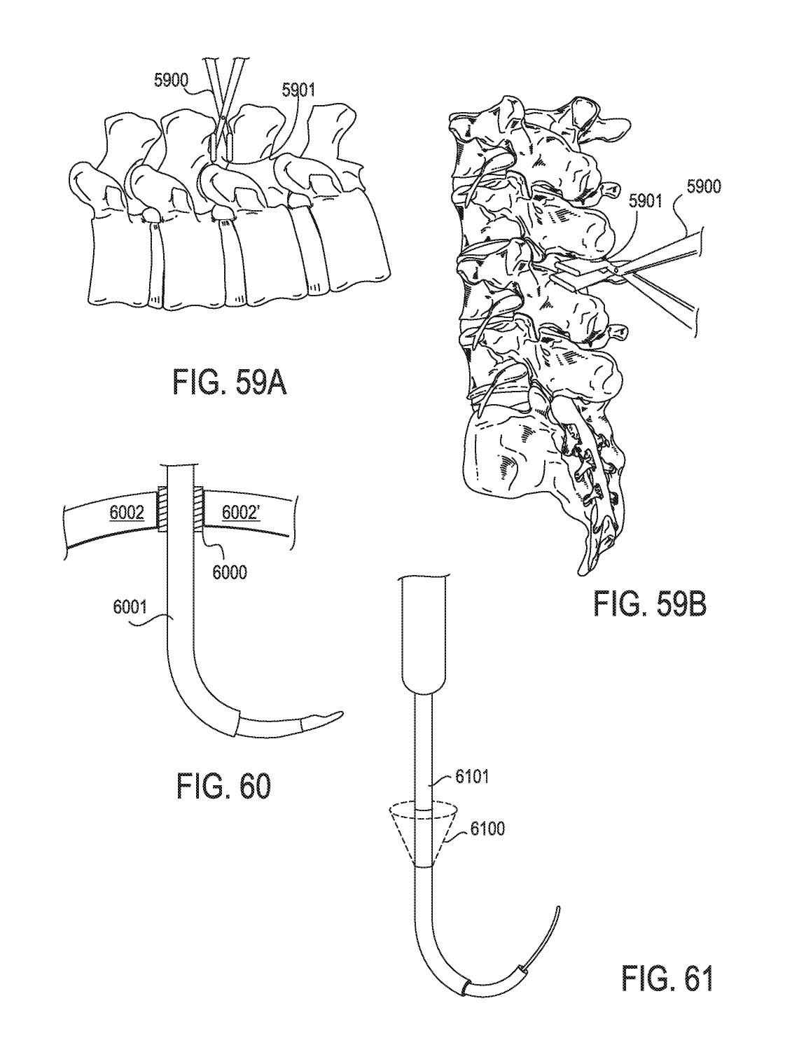

In some embodiments, an interlaminar spreader 5900, for example as shown in FIG. 59, may be used to distract or open the interlaminar window 5901, thereby creating additional space at the level of the spine. An interlaminar spreader may reduce the impact of a bulging disc, a disc herniation, a collapsed disc, and/or spondylolisthesis on the ability to access the spine. In some embodiments, as shown in FIGS. 60 and 61, an interlaminar spreader or distractor may be coupled to or incorporated with the probe device as described above. For example, as shown in FIG. 60, the spreader or distractor device 6000 may couple to the rigid shaft 6001 portion of the probe. The distractor may include an expandable element such that the element may be placed in the interlaminar window between the lamina 6002 and 6002′ of the spine in a collapsed configuration, and then expanded to a deployed or expanded configuration to open up the level of the spine. In some embodiments, the device may be an inflatable balloon, such as a high pressure balloon. As shown, the expandable device may be cannulated such that the probe may be removed from the spine while the device stays in place. In some embodiments, the neural localization devices and the tissue modification devices may be placed into the spine through the spreader/distractor device while the device holds the portion of the spine in a flexed or open configuration.

As shown in FIG. 61, a distractor device 6100 may be coupled to the rigid shaft portion 6101 of the probe. The distractor may be rotated or slid down the length of the probe. The distractor may be tapered or conically shaped such that as the device is moved down the length of the probe, it will move the lamina further and further apart. As described above, the expandable device may be cannulated such that the probe may be removed from the spine while the device stays in place. In some embodiments, the neural localization devices and the tissue modification devices may be placed into the spine through the spreader/distractor device while the device holds the portion of the spine in a flexed or open configuration.

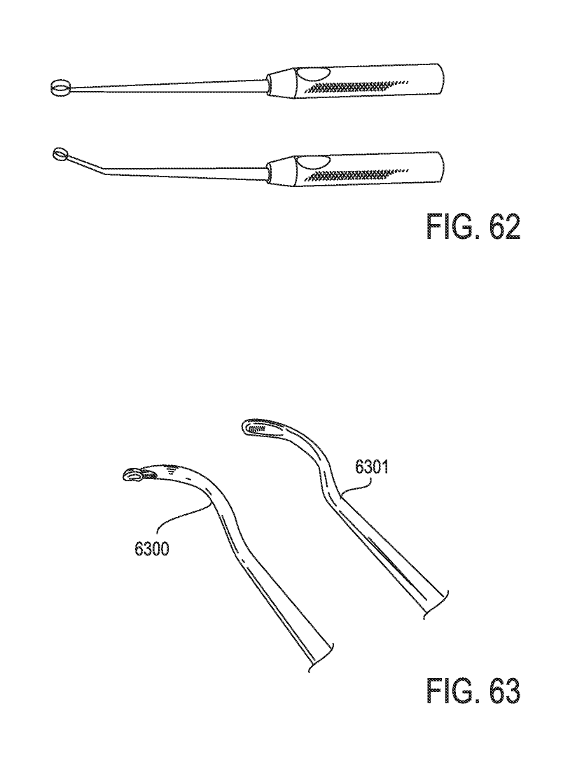

In some embodiments, it may be necessary to remove tissue, such as bone or ligament from the spinal level to be treated in order to obtain access with the access tools described above. In some embodiments, a surgeon may utilize a Rongeur or curette tool to remove tissue. In particular they may remove ligament or bony tissue to create space in the lateral recess and/or to increase foraminal height. As shown in FIG. 62, a curette tool is a surgical instrument shaped like a scoop to remove tissue from a body cavity. In some embodiments, a Rongeur or a curette (as shown in FIG. 63) may be incorporated on the distal end of the access probe. An access probe 6300 as described above is shown on the left of FIG. 63, and a curette tipped probe 6301 is shown on the right. In some embodiments, the curette may be configured to remove tissue in a single direction. For example, the curette may not remove tissue as the probe is pushed into the spine, and may only remove tissue as the probe tip is pulled back. The probe may be an up-biting probe or a down-biting probe. In some embodiments, the access probe may include a drill on its distal end configured to drill and remove tissue. In some embodiments, the curette probe may be cannulated to receive a pullwire and/or suction and irrigation. In some embodiments, the cutting tool may be configured to identify neural tissue. For example, stimulation electrodes may be coupled to the distal end of the tool.

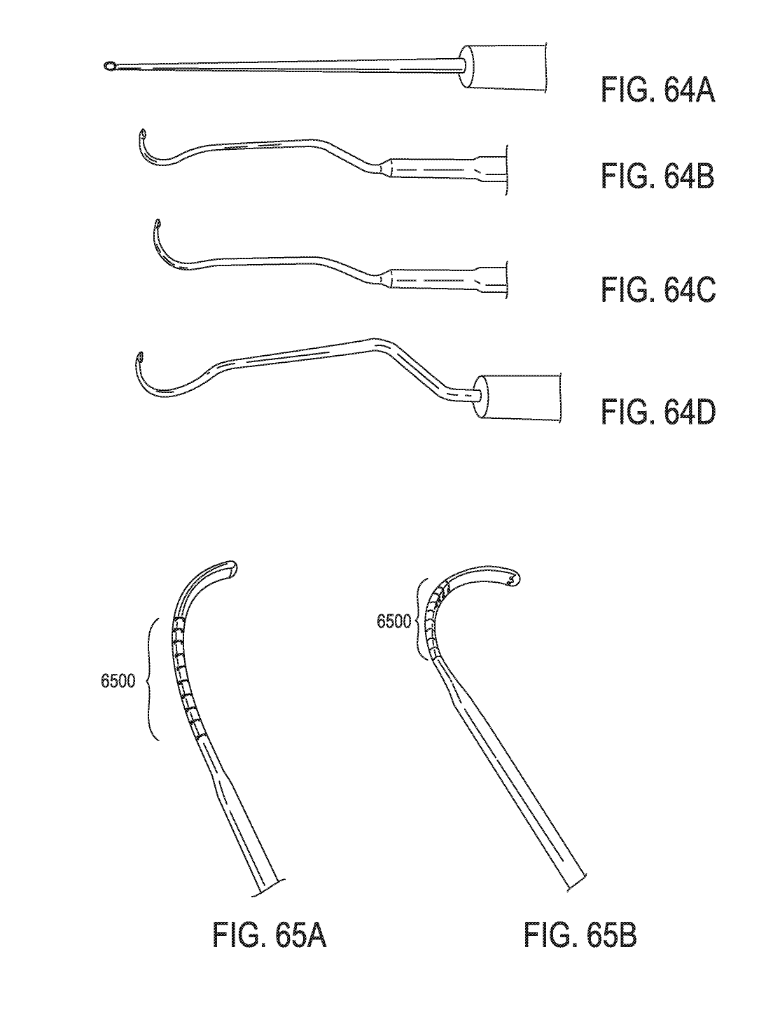

As shown in FIGS. 64A to 64D, the geometry of the distal end of the curette probe may be one of several suitable geometries. In some embodiments, specific cutting tools may be used to cut specific tissue within the spine. For example, the system may include a lateral recess tool sized and configured to remove tissue from the lateral recess, central canal tool sized and configured to remove tissue from the central canal, and/or laminotomy tool sized and configured to remove tissue from the lamina and/or create a laminotomy.

In some embodiments, as shown in FIGS. 65A and 65B, the distal end of the access probe may be steerable and/or articulating. For example, as shown in FIG. 65A, the distal end may have a shallow radius curve. As shown in FIG. 6B, the curve may be tightened. In some embodiments, this steering/articulation may be accomplished through the use of pull wires (not shown) coupled to the distal end of the probe. As shown in the figures, the distal end may be segmented 6500 such that each segment may move with respect to adjacent segments. The articulating probe may be used to move the probe in the lateral/medial directions, the caudal/cephalad directions, and/or in the ventral/dorsal directions. In some embodiments, the probe may include a locking mechanism such that the probe may be locked in position and become rigid in that position. In some embodiments, the articulating probe may be configured to identify neural tissue. For example, stimulation electrodes may be coupled to the distal end of the probe. In some alternative embodiments, the probe may include a visualization camera (e.g. fiber optic) or endoscope such that the neural tissue may be visualized.

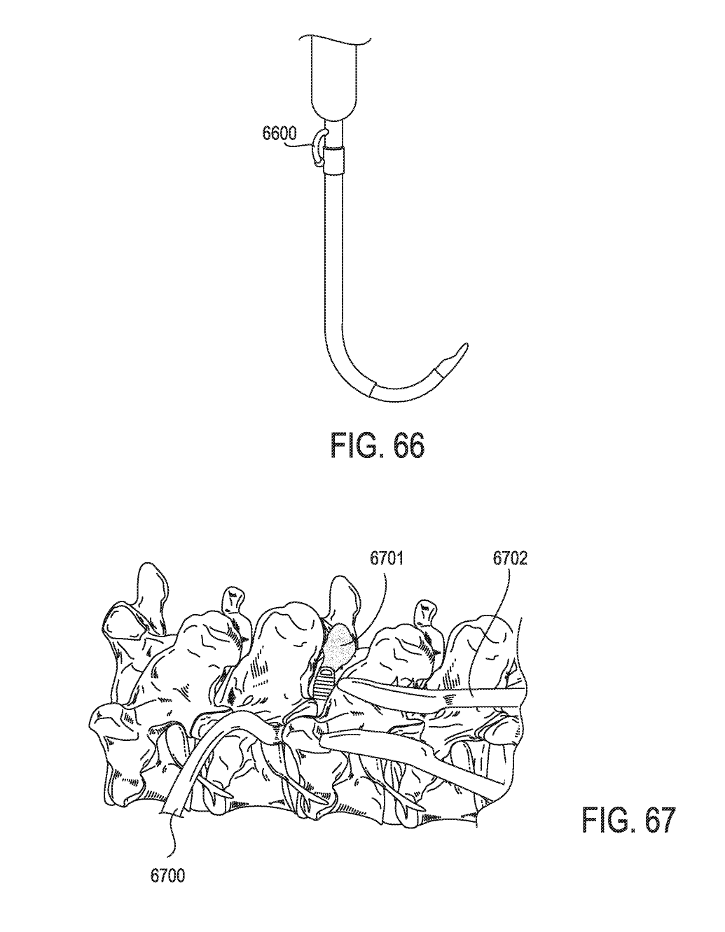

In some embodiments, once the probe is in position, a guidewire may be thread through the probe and deployed through the neural foramen of the spine. Once the guidewire is in position, the probe may be removed. In an alternative embodiment, the guidewire may be preloaded in the probe prior to gaining access with the probe. For example, the probe may be provided to the user (e.g. scrub tech and/or surgeon) with a pre-loaded guidewire, as shown in FIG. 66. The distal tip of the guidewire may be locked within the probe and then may be unlocked (via locking mechanism 6600 for example), once the probe is in position, and deployed through the foramen. In some embodiments, the proximal end of the guidewire may extend from the probe. This length of the guidewire may be contained in some fashion. For example, the length of the wire may be wound around a spool or held within a container.

In some embodiments, a probe may be used prior to gaining access to the spine. For example, the probe may be configured to be a measuring device to determine the size of the neural foramen and/or to determine the degree of stenosis, particularly of the foramen. As shown in FIG. 67, the probe device 6700 may be used to deliver a contrast agent 6701 into the foramen. As shown, 6702 are spinal retractors. As viewed under fluoroscopy, if the contrast agent disperses into and out of the foramen, as shown, it may then be determined that the foramen is relatively non-stenotic. Alternatively, if the agent cannot leave the foramen, it may then be determined that the foramen is highly stenotic. In some embodiments, the contrast agent may be viewed to determine what size cutting tool should be used for the procedure. Alternatively, the contrast agent may be viewed to determine if the decompression of the foramen was successful.

In some embodiments, any of the tools described herein, may be reusable instruments. In particular, the access probe, curette probe, and/or contrast agent delivery probe may be useful as reusable tools. In this example, the reusable tool may always be available for a surgeon on a tray of conventional reusable, sterilizable instruments such as Rongeurs and curettes, for example. For example, the probe may be kept on a conventional laminotomy tray. In some embodiments, the probe may be used to deliver irrigation fluids, steroids, and/or haemostatic agents.

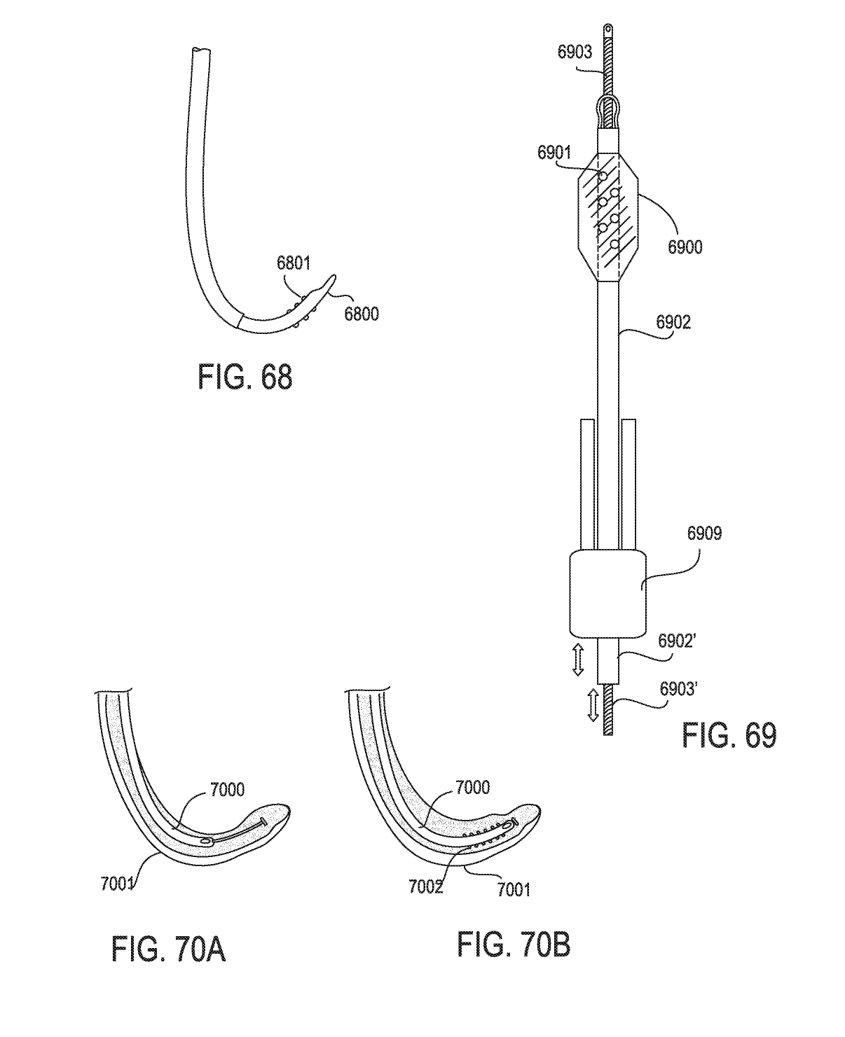

For example, as shown in FIG. 68, the catheter or inner cannula 6800 of the probe may include stimulation electrodes 6801. In some embodiments, the electrode may include a cathode array and an anode array, such that they may be used in a bipolar configuration. Alternatively, the electrodes may be used in a monopolar configuration.

In some embodiments, a neural localization component 6900 may be delivered into the spine over the probe, once the probe is deployed within the spine. As shown in FIG. 69, a neural localization element may include stimulation electrodes. In some embodiments, the electrode may include a cathode array and an anode array, such that they may be used in a bipolar configuration. Alternatively, the electrodes may be used in a monopolar configuration. Further, as shown, the neural localization element may include wings that extend beyond the diameter of the probe. The wings 6901 may prevent the stimulation current from wrapping around the neural localization device. For example, if the electrodes on the top of the device are stimulated, the current may wrap around to the bottom electrodes and may stimulate the nerve. A wider device may prevent the current from wrapping around the device. Alternatively, the electrodes may be positioned along the center of the probe also to prevent current wrapping. As shown, the neural localization device 6900 may extend over the outer cannula 6902 of the probe. Alternatively, the neural localization device may extend over the inner cannula or catheter 6903 of the probe. In some embodiments, as described above, the electrodes (and/or wings) may be directly coupled to the inner cannula. As shown the neural localization element may be coupled to the proximal handle portion 6904 such that a user may separately deploy the inner cannula with a first plunger or slider 6903′, and then deploy the neural localization device with a second plunger or slider 6900′.

The embodiment of FIGS. 70A and 70B is similar to the embodiment described above, however, the deployable component 7000 may be deployed down the center of the probe 7001. For example the deployable component may act as a wedge and push the probe into a wider or expanded configuration. The width of the expanded probe may prevent the current from wrapping around the device. As shown, the electrodes 7002 may be coupled to the outer portion of the device. Alternatively, they may be coupled to the center deployable portion. Due to the tightness of a stenotic foramen, it may be easier to deploy a wedge type device into the probe than pushing in a wide probe.

The examples and illustrations included herein show, by way of illustration and not of limitation, specific embodiments in which the subject matter may be practiced. Other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A probe device for positioning a wire around a target tissue, the device comprising:
a rigid outer cannula having a curved distal region;
a flexible inner cannula slideably disposed within the rigid outer cannula, wherein the inner cannula is configured to assume a curved shape when extended distally from the outer cannula;
a safety retainer extending distally; and
a distal tip at the end of the inner cannula, the distal tip coupled to the safety retainer.

2. The device of claim 1, wherein the distal tip comprises a port for exit of a wire through the distal tip.

3. The device of claim 1, wherein the safety retainer extends proximally to distally along with the inner cannula.

4. The device of claim 1, wherein at least a portion of the safety retainer is held slack within the device.

5. The device of claim 1, wherein the safety retainer is secured to the probe near the proximal end region of the safety retainer.

6. The device of claim 1, wherein the safety retainer comprises a cable extending within a safety retainer lumen within a cannula wall of the inner cannula.

7. The device of claim 4, wherein the cable is slideably disposed within the safety retainer lumen within the cannula wall of the inner cannula.

8. The device of claim 7, wherein the safety retainer comprises a loop of material extending within a first safety retainer lumen extending through the cannula wall, through the distal tip, and extending within a second safety retainer lumen extending through the cannula wall.

9. The device of claim 8, wherein the loop of cable is slack within the safety retainer lumens of the inner cannula.

10. The device of claim 1, wherein the safety retainer comprises a cable disposed within a central lumen of the inner cannula.

11. The device of claim 10, wherein the cable is slideably disposed within the central lumen of the inner cannula.

12. The device of claim 10, wherein the cable is slack within the central lumen of inner cannula.

13. The device of claim 1, wherein the cable is Nitinol.

14. The device of claim 1, wherein the safety retainer comprises a loop of material extending within a central lumen of the inner cannula, through the distal tip, and extending back within the central lumen of the inner cannula.

15. The device of claim 1, further comprising a longitudinally extending shape memory member.

16. The device of claim 15, wherein the shape memory member is a tubular member disposed within a central lumen within a cannula wall of the inner cannula.

17. The device of claim 15, wherein the shape memory member is a tubular member disposed within a central lumen of the inner cannula adjacent to the safety retainer also within the central lumen.

18. The device of claim 15, wherein the shape memory member is a Nitinol tube.

19. A probe device for positioning a wire around a target tissue, the probe device comprising:
a rigid outer cannula having a curved distal region;
a flexible inner cannula slideably disposed within the rigid outer cannula, wherein the inner cannula is configured to assume a curved shape when extended distally from the outer cannula;
a distal tip at the distal end of the inner cannula; and
a safety retainer cable coupled to the distal tip and secured to the probe proximally from the distal end of the safety retainer; wherein the safety retainer cable extends proximally from the distal tip and is slack.

20. A probe device for positioning a wire around a target tissue, the device comprising:
an outer cannula having a curved distal region;
an inner member slideably disposed within the outer cannula, wherein the inner member comprises:
an elongate body;
a longitudinally extending support member disposed inside the elongate body, and
a longitudinally extending tubular body disposed inside the elongate body, configured to assume a curved shape when extended distally from the outer cannula, and configured to pass a wire, and
a distal tip coupled to the support member.

21. The device of claim 20, wherein the elongate body comprises a central lumen configured to receive the tubular body and at least one support member lumen to receive the support member.

22. The device of claim 20, wherein the elongate body comprises a single central lumen and the support member and the tubular body are disposed inside the central lumen of the elongate body.

23. The device of claim 20, wherein the solid support members are curved.

24. The device of claim 20, wherein the solid support members comprises a shape memory alloy.

25. The device of claim 20, wherein the tubular body is a shape memory material.

26. The device of claim 25, wherein the tubular body is Nitinol.

27. The device of claim 25, wherein the tubular body has a curved distal end.

28. The device of claim 20, wherein the tubular body is flanked by solid support members.

29. A probe device for positioning a wire around a target tissue, the device comprising:
an outer cannula having a curved distal region sized and configured to be advanced towards a lateral recess and a neural foramen in a patient;
an inner cannula slideably disposed within the outer cannula, wherein the inner cannula is configured to assume a curved shape when extended distally from the outer cannula and configured to extend through the neural foramen and at least partially around an anterior portion of a facet joint and posterior to a spinal disc; and
a proximal handle comprising a hand grip region coupled to the outer cannula and a plunger region coupled to the inner cannula.

30. A probe device for positioning a wire around a target tissue, the device comprising:
an outer cannula having a curved distal region;
an inner cannula slideably disposed within the outer cannula, wherein the inner cannula is configured to assume a curved shape when extended distally from the outer cannula; and
a proximal handle comprising a hand grip region coupled to the outer cannula and a plunger region coupled to the inner cannula; wherein the proximal end of the plunger region comprises a funneled port for insertion of a wire through the probe.
